SEQUENCE LISTING (86 sequences)

SEQ ID NO: 1 - DNA, 28 bp, synthetic construct
  source 1..28: mol_type = other DNA
tgacagctagctcagtcctagggattgt

SEQ ID NO: 2 - DNA, 28 bp, synthetic construct
  source 1..28: mol_type = other DNA
tgacrgctagctcagtcctdgnnayngt

SEQ ID NO: 3 - DNA, 28 bp, synthetic construct
  source 1..28: mol_type = other DNA
tgacannnnnnnnnnnnnnagggattgt

SEQ ID NO: 4 - DNA, 26 bp, synthetic construct
  source 1..26: mol_type = other DNA
nnnnnnawwwwwtttttaaaaaarnr

SEQ ID NO: 5 - DNA, 26 bp, synthetic construct
  source 1..26: mol_type = other DNA
wwwwwwwwwwwwwwwwwwwwwwwwww

SEQ ID NO: 6 - DNA, 26 bp, synthetic construct
  source 1..26: mol_type = other DNA
wwwhdwhdwhdwhdwhdwhdwhdwww

SEQ ID NO: 7 - skipped

SEQ ID NO: 8 - DNA, 26 bp, synthetic construct
  source 1..26: mol_type = other DNA
vvvvssssvvnnnnvsssvsssvsss

SEQ ID NO: 9 - DNA, 26 bp, synthetic construct
  source 1..26: mol_type = other DNA
ssssssssssssssssssssssssss

SEQ ID NO: 10 - DNA, 31 bp, synthetic construct
  source 1..31: mol_type = other DNA
ttagcgaagaaaatggtttgttatagtcgaa

SEQ ID NO: 11 - DNA, 31 bp, synthetic construct
  source 1..31: mol_type = other DNA
ttgacrsyknnnnsnnnnnnwgggattgtnn

SEQ ID NO: 12 - DNA, 643 bp, synthetic construct
  source 1..643: mol_type = other DNA
tttacggctagctcagccctaggtattatgctagcgaattcattaaagaggagaaaggtaccatggggcccgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggtggcggaggtagccaaaaagttgcgcagctgaaaaaccgtgttgcgtacaaactgaaagaaaacgcgaagctggagaacatcgtggcgcgtctggaaaacgacaatgcgaacctggagaaagacattgcgaatctcgaaaaggacatcgcaaatctggaacgtgacgttgcgcgttaagcggccgccacgcaaaaaaccccgcttcggcggggttttttcgc

SEQ ID NO: 13 - DNA, 577 bp, synthetic construct
  source 1..577: mol_type = other DNA
tttacggctagctcagccctaggtattatgctagcgaattcattaaagaggagaaaggtaccatgtcccatcagaaaattattcaggatcttatcgcatggattgacgagcatattgaccagccgcttaacattgatgtagtcgcaaaaaaatcaggctattcaaagtggtacttgcaacgaatgttccgcacggtgacgcatcagacgcttggcgattacattcgccaacgccgcctgttactggccgccgttgagttgcgcaccaccgagcgtccgatttttgatatcgcaatggacctgggttatgtctcgcagcagaccttctcccgcgttttcgcgcggcagtttgatcgcactcccgcggattatcgccaccgcctgggtggcggaggtagcaacaccgttaaagaactgaaaaactacatccaggagctggaagagcgtaacgctgaactcaaaaacctgaaggaacacctgaaattcgcaaaagcggaactggaattcgaactggcggctcacaaattcgagtaaggcgcgcccacgcaaaaaaccccgcttcggcggggttttttcgc

SEQ ID NO: 14 - DNA, 1381 bp, synthetic construct
  source 1..1381: mol_type = other DNA
tttacggctagctcagccctaggtattatgctagcgaattcattaaagaggagaaaggtaccatggggcccgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggtggcggaggtagcacgcgtgtgcctttgtatggttttacttcgatttgtggaagaagacctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggagacggttgggtcaacgtcggtggttgccgttgttttcccgtctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaagtaaggcgcgcccacgcaaaaaaccccgcttcggcggggttttttcgc

SEQ ID NO: 15 - DNA, 993 bp, synthetic construct
  source 1..993: mol_type = other DNA
tttacggctagctcagccctaggtattatgctagcgaattcattaaagaggagaaaggtaccatgtcccatcagaaaattattcaggatcttatcgcatggattgacgagcatattgaccagccgcttaacattgatgtagtcgcaaaaaaatcaggctattcaaagtggtacttgcaacgaatgttccgcacggtgacgcatcagacgcttggcgattacattcgccaacgccgcctgttactggccgccgttgagttgcgcaccaccgagcgtccgatttttgatatcgcaatggacctgggttatgtctcgcagcagaccttctcccgcgttttcgcgcggcagtttgatcgcactcccgcggattatcgccaccgcctgggtggcggaggtagcatgggtgggggcgcgccaactcaagacgaattcacccaactctcccaatcaatcgccgagttccacacgtaccaactcggtaacggccgttgctcatctctcctagctcagcgaatccacgcgccgccggaaacagtatggtccgtggtgagacgtttcgataggccacagatttacaaacacttcatcaaaagctgtaacgtgagtgaagatttcgagatgcgagtgggatgcacgcgcgacgtgaacgtgataagtggattaccggcgaatacgtctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaactaagcggccgcccgcaaaaaaccccgcttcggcggggttttttcgc

SEQ ID NO: 16 - DNA, 747 bp, synthetic construct
  source 1..747: mol_type = other DNA
tttacggctagctcagccctaggtattatgctagcgaattcattaaagaggagaaaggtaccatggggcccgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggtggcggaggtagcatgaagcgcgatcatcatcaccaccaccaccaggataaaaagacgatgatgatgaatgaggaagatgatggaaacgggatggacgaattgctggcagtgctgggatataaggtgcgttcgtccgaaatggcagatgttgctcagaaattggagcagttagaagtaatgatgagtaacgttcaagaagatgatctttcacagttagcgaccgaaactgtccactacaaccctgctgagctttacacttggttggactccatgcttaccgatcttaactgacgcaaaaaaccccgcttcggcggggttttttcgc

SEQ ID NO: 17 - DNA, 1470 bp, synthetic construct
  source 1..1470: mol_type = other DNA
tttacggctagctcagccctaggtattatgctagcgaattcattaaagaggagaaaggtaccatgtcccatcagaaaattattcaggatcttatcgcatggattgacgagcatattgaccagccgcttaacattgatgtagtcgcaaaaaaatcaggctattcaaagtggtacttgcaacgaatgttccgcacggtgacgcatcagacgcttggcgattacattcgccaacgccgcctgttactggccgccgttgagttgcgcaccaccgagcgtccgatttttgatatcgcaatggacctgggttatgtctcgcagcagaccttctcccgcgttttcgcgcggcagtttgatcgcactcccgcggattatcgccaccgcctgggtggcggaggtagcatggcagcctccgacgaggtaaatcttattgagagtcgtaccgtcgttcccttgaatacttgggtgttgatctcgaatttcaaggtcgcgtacaatatcttacgccgcccggatggaacctttaaccgtcaccttgcagaatatctggaccgcaaagttacagcaaatgctaatccagttgacggtgttttcagttttgacgtgctgattgatcgccgtatcaaccttctgtcccgtgtctatcgtcctgcttacgccgatcaggagcaacctccatccattctggatctggaaaaaccagtggatggggacattgtccctgtcatcctttttttccacggggggtcgttcgcccactcgtccgccaacagtgcgatctacgacactttatgtcgtcgtcttgtcggtctttgcaaatgcgtggtcgtttccgtgaattaccgtcgcgctccggagaacccctacccatgtgcctacgacgacggatggattgcgttaaattgggttaattcacgtagctggctgaaaagcaagaaagattcgaaggttcacatttttttagcgggcgattcttcaggagggaacatcgctcataatgtcgcattgcgtgcaggagagtctggcatcgatgttctgggcaacattttactgaacccgatgtttggggggaacgagcgcacagaatccgagaaaagcttggacgggaagtatttcgtgactgttcgcgatcgtgactggtattggaaagcgttcttgcccgagggagaggaccgcgagcaccccgcatgcaaccccttttcacctcgcggaaaatcgctggagggggtcagtttcccaaaatctttagtcgtagtagctggcctggatctgatccgtgattggcaacttgcgtatgctgaaggccttaagaaggctggtcaagaagtaaagctgatgcacttagagaaagctacggttggcttttatctgttaccaaataacaatcacttccataatgtgatggatgagatctccgctttcgttaatgcggaatgctgacgcaaaaaaccccgcttcggcggggttttttcgc

SEQ ID NO: 18 - DNA, 890 bp, synthetic construct
  source 1..890: mol_type = other DNA
tttacggctagctcagtcctaggtatagtgctagcctagagaaagaggagaaatactagatgttggatatgggacaagatcggccgatcgatggaagtggggcacccggggcagacgacacacgcgttgaggtgcaaccgccggcgcagtgggtcctcgacctgatcgaggccagcccgatcgcatcggtcgtgtccgatccgcgtctcgccgacaatccgctgatcgccatcaaccaggccttcaccgacctgaccggctattccgaagaagaatgcgtcggccgcaattgccgattcctggcaggttccggcaccgagccgtggctgaccgacaagatccgccaaggcgtgcgcgagcacaagccggtgctggtcgagatcctgaactacaagaaggacggcacgccgttccgcaatgccgtgctcgttgcaccgatctacgatgacgacgacgagcttctctatttcctcggcagccaggtcgaagtcgacgacgaccagcccaacatgggcatggcgcgccgcgaacgcgccgcggaaatgctcaagacgctgtcgccgcgccagctcgaggttacgacgctggtggcatcgggcttgcgcaacaaggaagtggcggcccggctcggcctgtcggagaaaaccgtcaagatgcaccgcgggctggtgatggaaaagctcaacctgaagaccagcgccgatctggtgcgcattgccgtcgaagccggaatctaaggatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 19 - DNA, 846 bp, synthetic construct
  source 1..846: mol_type = other DNA
ggtagcctttagtccatgttgacggtgaagagtatcagagggattgtgctagcgaattcattaaagaggagaaaggtaccatggcgagtagcgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaaggatccaaactcgagtaaggatctgtgctttttttaacgcatgagaaagcccccggaagatcaccttccgggggcttttttattgcgc

SEQ ID NO: 20 - DNA, 20 bp, synthetic construct
  source 1..20: mol_type = other DNA
aactctcacacgtggctgca

SEQ ID NO: 21 - DNA, 20 bp, synthetic construct
  source 1..20: mol_type = other DNA
aactctcacacgtggctgca

SEQ ID NO: 22 - DNA, 20 bp, synthetic construct
  source 1..20: mol_type = other DNA
aactctcacacgtggctgca

SEQ ID NO: 23 - DNA, 20 bp, synthetic construct
  source 1..20: mol_type = other DNA
cgccgaatgctctagcggga

SEQ ID NO: 24 - DNA, 20 bp, synthetic construct
  source 1..20: mol_type = other DNA
cacacctaagtcaggattgt

SEQ ID NO: 25 - DNA, 20 bp, synthetic construct
  source 1..20: mol_type = other DNA
catatctctgacctgatcga

SEQ ID NO: 26 - DNA, 20 bp, synthetic construct
  source 1..20: mol_type = other DNA
cacataaaaaccgctgacta

SEQ ID NO: 27 - DNA, 20 bp, synthetic construct
  source 1..20: mol_type = other DNA
aactctcacacgtggctgca

SEQ ID NO: 28 - DNA, 20 bp, synthetic construct
  source 1..20: mol_type = other DNA
agaaacagtaaaaactttca

SEQ ID NO: 29 - DNA, 20 bp, synthetic construct
  source 1..20: mol_type = other DNA
tcggctcacttatgcacggc

SEQ ID NO: 30 - DNA, 20 bp, synthetic construct
  source 1..20: mol_type = other DNA
tccctgaccctcggctcact

SEQ ID NO: 31 - DNA, 20 bp, synthetic construct
  source 1..20: mol_type = other DNA
accctttccttccctgaccc

SEQ ID NO: 32 - DNA, 20 bp, synthetic construct
  source 1..20: mol_type = other DNA
ttcctctcctaccctttcct

SEQ ID NO: 33 - DNA, 26 bp, synthetic construct
  source 1..26: mol_type = other DNA
ccggcggcggcggctgccgcgcggcg

SEQ ID NO: 34 - DNA, 26 bp, synthetic construct
  source 1..26: mol_type = other DNA
ccgcgggcggccgctgccggggcgcg

SEQ ID NO: 35 - DNA, 26 bp, synthetic construct
  source 1..26: mol_type = other DNA
ccgcgggcggccgctgccggggcgcg

SEQ ID NO: 36 - DNA, 26 bp, synthetic construct
  source 1..26: mol_type = other DNA
ccgcgggcggccgctgccggggcgcg

SEQ ID NO: 37 - DNA, 26 bp, synthetic construct
  source 1..26: mol_type = other DNA
ccgcgggcggccgctgccggggcgcg

SEQ ID NO: 38 - DNA, 26 bp, synthetic construct
  source 1..26: mol_type = other DNA
ccgcgggcggccgctgccggggcgcg

SEQ ID NO: 39 - DNA, 26 bp, synthetic construct
  source 1..26: mol_type = other DNA
ccgcgggcggccgctgccggggcgcg

SEQ ID NO: 40 - DNA, 26 bp, synthetic construct
  source 1..26: mol_type = other DNA
ccgcgggcggccgctgccggggcgcg

SEQ ID NO: 41 - DNA, 26 bp, synthetic construct
  source 1..26: mol_type = other DNA
ccgcgggcggccgctgccggggcgcg

SEQ ID NO: 42 - DNA, 20 bp, synthetic construct
  source 1..20: mol_type = other DNA
tcggtagcctttagtccatg

SEQ ID NO: 43 - DNA, 20 bp, synthetic construct
  source 1..20: mol_type = other DNA
tcggtagcctttagtccatg

SEQ ID NO: 44 - DNA, 20 bp, synthetic construct
  source 1..20: mol_type = other DNA
tcggtagcctttagtccatg

SEQ ID NO: 45 - DNA, 20 bp, synthetic construct
  source 1..20: mol_type = other DNA
tcggtagcctttagtccatg

SEQ ID NO: 46 - DNA, 26 bp, synthetic construct
  source 1..26: mol_type = other DNA
ccggccccccccgctgccgcgggccg

SEQ ID NO: 47 - DNA, 26 bp, synthetic construct
  source 1..26: mol_type = other DNA
ccggccccccccgctgccgcgggccg

SEQ ID NO: 48 - DNA, 26 bp, synthetic construct
  source 1..26: mol_type = other DNA
ccggccccccccgctgccgcgggccg

SEQ ID NO: 49 - DNA, 26 bp, synthetic construct
  source 1..26: mol_type = other DNA
ccggccccccccgctgccgcgggccg

SEQ ID NO: 50 - DNA, 35 bp, synthetic construct
  source 1..35: mol_type = other DNA
ttgacagttttacgatgtgttgggattgtgctagc

SEQ ID NO: 51 - DNA, 35 bp, synthetic construct
  source 1..35: mol_type = other DNA
ttgacacttccggcacgaaaagggattgtgctagc

SEQ ID NO: 52 - DNA, 35 bp, synthetic construct
  source 1..35: mol_type = other DNA
ttgacgcctccttctttcttagggattgtgctagc

SEQ ID NO: 53 - DNA, 35 bp, synthetic construct; identical to SEQ ID NO: 52
  source 1..35: mol_type = other DNA

SEQ ID NO: 54 - DNA, 35 bp, synthetic construct; identical to SEQ ID NO: 52
  source 1..35: mol_type = other DNA

SEQ ID NO: 55 - DNA, 35 bp, synthetic construct; identical to SEQ ID NO: 52
  source 1..35: mol_type = other DNA

SEQ ID NO: 56 - DNA, 35 bp, synthetic construct; identical to SEQ ID NO: 52
  source 1..35: mol_type = other DNA

SEQ ID NO: 57 - DNA, 35 bp, synthetic construct; identical to SEQ ID NO: 52
  source 1..35: mol_type = other DNA

SEQ ID NO: 58 - DNA, 35 bp, synthetic construct; identical to SEQ ID NO: 52
  source 1..35: mol_type = other DNA

SEQ ID NO: 59 - DNA, 35 bp, synthetic construct
  source 1..35: mol_type = other DNA
ttgacgctgtattcaggcaaagggattgtgctagc

SEQ ID NO: 60 - DNA, 35 bp, synthetic construct
  source 1..35: mol_type = other DNA
ttgacagtgcgtacgcagggagggattgtgctagc

SEQ ID NO: 61 - DNA, 35 bp, synthetic construct
  source 1..35: mol_type = other DNA
ttgacggtgaagagtatcagagggattgtgctagc

SEQ ID NO: 62 - DNA, 35 bp, synthetic construct
  source 1..35: mol_type = other DNA
ttgacagctcagtgagtagtagggattgtgctagc

SEQ ID NO: 63 - DNA, 35 bp, synthetic construct
  source 1..35: mol_type = other DNA
ttgacaaagctatggccggcagggattgtcacagc

SEQ ID NO: 64 - DNA, 35 bp, synthetic construct; identical to SEQ ID NO: 63
  source 1..35: mol_type = other DNA

SEQ ID NO: 65 - DNA, 35 bp, synthetic construct; identical to SEQ ID NO: 63
  source 1..35: mol_type = other DNA

SEQ ID NO: 66 - DNA, 35 bp, synthetic construct; identical to SEQ ID NO: 63
  source 1..35: mol_type = other DNA

SEQ ID NO: 67 - DNA, 60 bp, synthetic construct
  source 1..60: mol_type = other DNA
cgattatagattgacrgctagctcagtcctdgnnayngtgctagcgaattcattaaagag

SEQ ID NO: 68 - DNA, 65 bp, synthetic construct
  source 1..65: mol_type = other DNA
cgattatagattgacttgacannnnnnnnnnnnnnagggattgtnnnagcgaattcattaaagag

SEQ ID NO: 69 - DNA, 82 bp, synthetic construct
  source 1..82: mol_type = other DNA
cgctagcacaatcccwnnnnnnsnnnnmrsygtcaagccgggagagctggttccattgcgattgcagccacgtgtgagagtt

SEQ ID NO: 70 - DNA, 82 bp, synthetic construct
  source 1..82: mol_type = other DNA
cgctagcacaatcccwnnnnnnsnnnnmrsygtcaacgccgcccggcagccggcgcgggcgctgcagccacgtgtgagagtt

SEQ ID NO: 71 - DNA, 82 bp, synthetic construct
  source 1..82: mol_type = other DNA
cgctagcacaatcccwnnnnnnsnnnnmrsygtcaacgccgcgcggcagccgccgccgccggtgcagccacgtgtgagagtt

SEQ ID NO: 72 - DNA, 60 bp, synthetic construct
  source 1..60: mol_type = other DNA
gctagctgtcaaynyttttttaaaaawwwwwtnnnnnnttgtgtccagaacgctccgtag

SEQ ID NO: 73 - DNA, 60 bp, synthetic construct
  source 1..60: mol_type = other DNA
gctagctgtcaawwwwwwwwwwwwwwwwwwwwwwwwwwttgtgtccagaacgctccgtag

SEQ ID NO: 74 - DNA, 60 bp, synthetic construct
  source 1..60: mol_type = other DNA
gctagctgtcaawwwhdwhdwhdwhdwwdwhwwhdwwwttgtgtccagaacgctccgtag

SEQ ID NO: 75 - DNA, 60 bp, synthetic construct
  source 1..60: mol_type = other DNA
gctagctgtcaannnnnnnnnnnnnnnnnnnnnnnnnnttgtgtccagaacgctccgtag

SEQ ID NO: 76 - DNA, 60 bp, synthetic construct
  source 1..60: mol_type = other DNA
gctagctgtcaabbbbssssbbnnnnbsssbsssbsssttgtgtccagaacgctccgtag

SEQ ID NO: 77 - DNA, 60 bp, synthetic construct
  source 1..60: mol_type = other DNA
gctagctgtcaassssssssssssssssssssssssssttgtgtccagaacgctccgtag

SEQ ID NO: 78 - DNA, 60 bp, synthetic construct
  source 1..60: mol_type = other DNA
gctagctgtcaasssssssssssssssssssssssssstgcagccacgtgtgagagttag

SEQ ID NO: 79 - DNA, 90 bp, synthetic construct
  source 1..90: mol_type = other DNA
gctcgtctcctcacttctcctwwwwwwwwwwwwwwwwwwwwccggccccccccgctgccgcgggccgttgacagctagctcagtcctagg

SEQ ID NO: 80 - DNA, 90 bp, synthetic construct
  source 1..90: mol_type = other DNA
gctcgtctcctcacttctcctnnnnnnnnnnnnnnnnnnnnccggccccccccgctgccgcgggccgttgacagctagctcagtcctagg

SEQ ID NO: 81 - DNA, 90 bp, synthetic construct
  source 1..90: mol_type = other DNA
gctcgtctcctcacttctcctssssssssssssssssssssccggccccccccgctgccgcgggccgttgacagctagctcagtcctagg

SEQ ID NO: 82 - DNA, 75 bp, synthetic construct
  source 1..75: mol_type = other DNA
cctctttaatgaattcgctnnnacaatcccwnnnnnnsnnnnmrsygtcaacatggactaaaggctacctataaa

SEQ ID NO: 83 - DNA, 35 bp, synthetic construct
  source 1..35: mol_type = other DNA
ttgacagctagctcagtcctagggattgtgctagc

SEQ ID NO: 84 - DNA, 26 bp, synthetic construct
  source 1..26: mol_type = other DNA
cgtcgtcttgaagttgcgattataga

SEQ ID NO: 85 - DNA, 20 bp, synthetic construct
  source 1..20: mol_type = other DNA
acggagcgttctggacacaa

SEQ ID NO: 86 - DNA, 35 bp, synthetic construct
  source 1..35: mol_type = other DNA
ttagcgaagaaaatggtttgttatagtcgaataaa

(19) United States
(12) Patent Application Publication
(10) Pub. No.: US 2026/0357250 A1
(43) Pub. Date: Nov. 12, 2026

(54) CONDITIONALLY RESPONSIVE CRISPRA USING PROTEIN-PROTEIN INTERACTION

(71) Applicant: University of Washington, Seattle, WA (US)

(72) Inventors: Ryan Cardiff, Seattle, WA (US); James M. Carothers, Seattle, WA (US); Jesse Zalatan, Seattle, WA (US); Diego Alba Burbano, Seattle, WA (US)

(73) Assignee: University of Washington, Seattle, WA (US)

(21) Appl. No.: 19/118,154

(22) PCT Filed: Oct. 25, 2023
(86) PCT No.: PCT/US2023/077771

§ 371 Date: Apr. 3, 2025

Related U.S. Application Data
(60) Provisional application No. 63/380,935, filed on Oct. 25, 2022.

(51) Int. Cl. C12N 15/11; C12N 9/22; C12N 15/63

(52) U.S. Cl. CPC C12N 15/111; C12N 9/226; C12N 15/635; C07K 2319/73; C07K 2319/80; C12N 2310/20

(57) ABSTRACT

High-performance promoters, and other high-performance components, for the construction of CRISPR-based synthetic gene regulatory networks (GRNs) and methods for engineering and using the promoters and components. Activatable promoters with lower basal and higher activated expression levels can be engineered with sequence-based design and in vivo high-throughput screening. The promoters achieve up to a 1000-fold dynamic range in an E. coli-based cell-free system (CFS) and constitute a 33-fold improvement in CRISPRa efficiency and enable implementation of large circuits not accessible with previous components, including six-layer deep and four-branch parallel cascades. The disclosed components enable assembly of novel input signal processing modules, including blue light responsive CRISPRa/i circuits and conditional CRISPRa based on small-molecule responsive protein-protein interactions. Multi-input processing networks can integrate the disclosed modules into a dynamic, two-input CRISPRa/i pulse generator.

### CROSS-REFERENCE TO RELATED APPLICATION

This PCT application claims the benefit of U.S. Provisional Patent Application No. 63/380,935 filed on Oct. 25, 2022, the content of which is incorporated by reference herein in its entirety.

### STATEMENT OF GOVERNMENT LICENSE RIGHTS

This invention was made with government support under Grant Nos. 62-2196 and EF-1935087, awarded by the National Science Foundation and Grant No. DE-AR00002387-1567, awarded by the U.S. Department of Energy. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3915-P1275WOUW_Sequence_Listing.xml. The XML file is 84,553 bytes; was created on Oct. 23, 2023; and is being submitted electronically via Patent Center with the filing of the specification.

### BACKGROUND

Gene regulatory networks (GRNs) of biological systems sense diverse environmental cues and respond to them through the coordinated expression of multiple genes. Synthetic biological systems have great potential in a variety of industrial applications, including but not limited to biosynthesis, biosensor applications, and biocomputational analysis. For example, the ability to construct complex GRNs can accelerate the development of cell-free systems (CFS) for investigating the limits of biological circuit design, building multiplexed molecular biosensors, deploying on-demand bioproduction platforms, and even facilitating the bottom-up construction of synthetic cells.

CFS are an attractive chassis for building synthetic biological systems as they allow for rapid prototyping of genetic parts and circuits, however, to continue building increasingly complex cell-free systems that can sense and respond to diverse inputs, new approaches for increasing the capabilities of synthetic GRNs are needed. GRN complexity is currently limited by an inability to routinely create high-performing components that can be interconnected with minimal signal degradation.

Accordingly, there is a need for improved synthetic GRNs, and components thereof, for use in a variety of applications, including but not limited to metabolic engineering and information processing applications. The present disclosure addresses these and other long-felt but unmet needs in the field.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In an aspect, the disclosure provides a promoter comprising a minimal promoter sequence and an upstream element (UP-element) sequence that comprises a nucleotide base mutation for a modified promoter-RNA polymerase interaction to configure the promoter for a low basal transcription and a high active transcription.

In embodiments, the UP-element sequence comprises a higher GC content relative to a GC content of an UP-element reference sequence.

In embodiments, the UP-element sequence comprises between 50% and 100% GC content.

In embodiments, the UP-element sequence comprises at least 80% identity with a sequence selected from SEQ ID NOs: 33-49; and the minimal promoter sequence comprises at least 80% identity with a sequence selected from SEQ ID NOs: 50-66.

In embodiments, the UP-element sequence comprises at least 90% identity with a sequence selected from SEQ ID NOs: 33-49; and the minimal promoter sequence comprises at least 90% identity with a sequence selected from SEQ ID NOs: 50-66.

In embodiments, the UP-element sequence comprises 100% identity with a sequence selected from SEQ ID NOs: 33-49; and the minimal promoter sequence comprises 100% identity with a sequence selected from SEQ ID NOs: 50-66.

In embodiments, a promoter further comprises a scaffold RNA (scRNA) target sequence configured to bind a guide RNA (gRNA) or single guide RNA (sgRNA) for localization of a Clustered Regularly Interspaced Short Palindromic Repeats activation (CRISPRa) complex to the promoter, wherein the CRISPRa complex comprises a deactivated Cas9 (dCas9) linked with a MS2 bacteriophage coat protein (MCP) polypeptide and a first heterodimer interaction domain.

In embodiments, the scRNA target sequence comprises a higher GC content relative to a GC content of a scRNA reference sequence.

In embodiments, the scRNA target sequence comprises at least 80% identity with a sequence selected from SEQ ID NOs: 20-32.

In embodiments, the scRNA target sequence comprises at least 90% identity with a sequence selected from SEQ ID NOs: 20-32.

In embodiments, the scRNA target sequence comprises 100% identity with a sequence selected from SEQ ID NOs: 20-32.

In embodiments, the promoter comprises at least 80% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

In embodiments, the promoter comprises at least 90% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

In embodiments, the promoter comprises 100% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

In an aspect, the disclosure provides a CRISPRa/i node, comprising: a promoter comprising a minimal promoter sequence and an upstream element (UP-element) sequence that comprises a nucleotide base mutation for a modified promoter-RNA polymerase interaction to configure the promoter for a low basal transcription and a high active transcription; a dCas9 polypeptide complexed, via a gRNA or a sgRNA, with a MCP polypeptide that is linked with a first heterodimer interaction domain; and a second heterodimer interaction domain that is linked with a SRY-related HMG-box (SoxS) domain; wherein the gRNA or the sgRNA is configured to interact with the promoter to localize the first heterodimer interaction domain to the promoter, and wherein the first heterodimer interaction domain is configured to conditionally interact with the second heterodimer interaction domain to localize the SoxS domain to the promoter.

In embodiments, a condition is associated with an interaction between the first heterodimer interaction domain and the second heterodimer interaction domain. When the condition is met, the interaction occurs and an activation complex is formed for activation of transcription via the SoxS domain.

In embodiments, the condition comprises irradiation of a matrix that comprises the CRISPRa/i node with a light that comprises a blue light element or contact of the matrix that comprises the CRISPRa/i node with a small molecule.

In embodiments, the condition comprises irradiation of the matrix with the light that comprises the blue light element and the method is performed using a EL222 light-responsive promoter system.

In embodiments, the first heterodimer interaction domain comprises a heterospecific synthetic coiled-coil peptide A (SYNZIP A) domain and the second heterodimer interaction domain comprises a heterospecific synthetic coiled-coil peptide B (SYNZIP B) domain or the first heterodimer interaction domain comprises a SYNZIP B domain and the second heterodimer interaction domain comprises a SYNZIP A domain; the first heterodimer interaction domain comprises an abortive infectivity (ABI) domain and the second heterodimer interaction domain comprises a pyrabactin resistance-like 1 (PYL1) domain or the first heterodimer interaction domain comprises a PYL1 domain and the second heterodimer interaction domain comprises an ABI domain; or the first heterodimer interaction domain comprises a gibberellin (GAI) domain and the second heterodimer interaction domain comprises a gibberellin insensitive dwarf 1 (GID1) domain or the first heterodimer interaction domain comprises a GID1 domain and the second heterodimer interaction domain comprises a GAI domain.

In embodiments, the UP-element sequence comprises between 50% and 100% GC content.

In embodiments, the UP-element sequence comprises at least 80% identity with a sequence selected from SEQ ID NOs: 33-49; and the minimal promoter sequence comprises at least 80% identity with a sequence selected from SEQ ID NOs: 50-66.

In embodiments, the UP-element sequence comprises at least 90% identity with a sequence selected from SEQ ID NOs: 33-49; and the minimal promoter sequence comprises at least 90% identity with a sequence selected from SEQ ID NOs: 50-66.

In embodiments, the UP-element sequence comprises 100% identity with a sequence selected from SEQ ID NOs: 33-49; and the minimal promoter sequence comprises 100% identity with a sequence selected from SEQ ID NOs: 50-66.

In embodiments, the CRISPRa/i node further comprises a scRNA target sequence configured to bind a gRNA or sgRNA for localization of a CRISPRa complex to the promoter, and the CRISPRa complex comprises a dCas9 linked with a MCP polypeptide and a first heterodimer interaction domain.

In embodiments, the scRNA target sequence comprises a higher GC content relative to a GC content of a scRNA target reference sequence.

In embodiments, the scRNA target sequence comprises at least 80% identity with a sequence selected from SEQ ID NOs: 20-32.

In embodiments, the scRNA target sequence comprises at least 90% identity with a sequence selected from SEQ ID NOs: 20-32.

In embodiments, the scRNA target sequence comprises 100% identity with a sequence selected from SEQ ID NOs: 20-32.

In embodiments, the promoter comprises at least 80% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

In embodiments, the promoter comprises at least 90% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

In embodiments, the promoter comprises 100% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

In an aspect, the disclosure provides a synthetic gene regulatory network (GRN) comprising at least a first CRISPRa/i node and a second CRISPRa/i node, wherein the first CRISPRa/i node comprises a first promoter and the second CRISPRa/i node comprises a second promoter, wherein the first CRISPRa/i node is configured for a sensing function and the second CRISPRa/i node is configured for a propagation function or a reporting function. In embodiments, the first promoter, the second promoter, or both, comprises a minimal promoter sequence and an upstream element (UP-element) sequence that comprises a nucleotide base mutation for a modified promoter-RNA polymerase interaction to configure the promoter for a low basal transcription and a high active transcription.

In embodiments, a first transcription product results from activation of transcription of the first CRISPRa/i node and is an input to the second CRISPRa/i node, and wherein a second transcription product results from activation of transcription of the second CRISPRa/i node and is an output of the second CRISPRa/i node, the synthetic GRN, or both.

In embodiments, the synthetic GRN comprises a feedback loop, a feedforward loop, a positive feedback, a negative feedback, a positive feedforward, and/or a negative feedforward.

In embodiments, the propagation function implements a decreasing strategy, a constant strategy, an increasing strategy, or a data-informed strategy.

In embodiments, the first CRISPRa/i node, the second CRISPRa/i node, or both, comprises: a dCas9 polypeptide complexed, via a gRNA or a sgRNA, with a MCP polypeptide that is linked with a first heterodimer interaction domain; and a second heterodimer interaction domain that is linked with a SoxS domain; wherein the gRNA or the sgRNA is configured to interact with the first promoter, the second promoter, or both to localize the first heterodimer interaction domain to the first promoter, the second promoter, or both, and wherein the first heterodimer interaction domain is configured to conditionally interact with the second heterodimer interaction domain to localize the SoxS domain to the first promoter, the second promoter, or both.

In embodiments, a condition is associated with an interaction between the first heterodimer interaction domain and the second heterodimer interaction domain. When the condition is met, the interaction occurs and an activation complex is formed for activation of transcription via the SoxS domain.

In embodiments, the condition comprises irradiation of a matrix that comprises the CRISPRa/i node with a light that comprises a blue light element or contact of the matrix that comprises the CRISPRa/i node with a small molecule.

In embodiments, the condition comprises irradiation of the matrix with the light that comprises the blue light element and the method is performed using a EL222 light-responsive promoter system.

In embodiments, the first heterodimer interaction domain comprises a SYNZIP A domain and the second heterodimer interaction domain comprises a SYNZIP B domain or the first heterodimer interaction domain comprises a SYNZIP B domain and the second heterodimer interaction domain comprises a SYNZIP A domain; the first heterodimer interaction domain comprises an ABI domain and the second heterodimer interaction domain comprises a PYL1 domain or the first heterodimer interaction domain comprises a PYL1 domain and the second heterodimer interaction domain comprises an ABI domain; or the first heterodimer interaction domain comprises a GAI domain and the second heterodimer interaction domain comprises a GID1 domain or the first heterodimer interaction domain comprises a GID1 domain and the second heterodimer interaction domain comprises a GAI domain.

In embodiments, the UP-element sequence comprises between 50% and 100% GC content.

In embodiments, the UP-element sequence comprises at least 80% identity with a sequence selected from SEQ ID NOs: 33-49; and the minimal promoter sequence comprises at least 80% identity with a sequence selected from SEQ ID NOs: 50-66.

In embodiments, the UP-element sequence comprises at least 90% identity with a sequence selected from SEQ ID NOs: 33-49; and the minimal promoter sequence comprises at least 90% identity with a sequence selected from SEQ ID NOs: 50-66.

In embodiments, the UP-element sequence comprises 100% identity with a sequence selected from SEQ ID NOs: 33-49; and the minimal promoter sequence comprises 100% identity with a sequence selected from SEQ ID NOs: 50-66.

In embodiments, the synthetic GRN further comprises a scRNA target sequence configured to bind the gRNA or the sgRNA for localization of a CRISPRa complex to the promoter, wherein the CRISPRa complex comprises the dCas9 polypeptide complexed, via the gRNA or the sgRNA, with the MCP polypeptide that is linked with the first heterodimer interaction domain.

In embodiments, the scRNA target sequence comprises a higher GC content relative to a GC content of a scRNA reference sequence.

In embodiments, the scRNA target sequence comprises at least 80% identity with a sequence selected from SEQ ID NOs: 20-32.

In embodiments, the scRNA target sequence comprises at least 90% identity with a sequence selected from SEQ ID NOs: 20-32.

In embodiments, the scRNA target sequence comprises 100% identity with a sequence selected from SEQ ID NOs: 20-32.

In embodiments, the first promoter, the second promoter, or both, comprises at least 80% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

In embodiments, the first promoter, the second promoter, or both, comprises at least 90% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

In embodiments, the first promoter, the second promoter, or both, comprises 100% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

In embodiments, the first promoter has a higher dynamic range of transcription and the second promoter has a higher absolute range of transcription.

In embodiments, the synthetic GRN is at least one, two, three, four, five, or six layers deep, and is at least one, two, three, or four branches wide.

In an aspect, the disclosure provides one or more nucleic acids comprising nucleotide sequences encoding one or more polypeptides, the gRNA or the sgRNA, one or more genes regulated by the CRISPRa/i node, a promoter, or any combination thereof.

In an aspect, the disclosure provides an E. coli CFS comprising polypeptides and a promoter.

In an aspect, the disclosure provides a genetically modified host cell comprising one or more nucleic acids encoding a promoter.

In embodiments, the genetically modified host cell is a prokaryotic cell or a eukaryotic cell.

In an aspect, the disclosure provides a synthetic biosensor, synthetic bioreactor, or other synthetic biological system comprising a synthetic GRN.

In an aspect, the disclosure provides a method for control of transcription, the method comprising: providing a promoter comprising a minimal promoter sequence and an upstream element (UP-element) sequence that comprises a nucleotide base mutation for a modified promoter-RNA polymerase interaction to configure the promoter for a low basal transcription and a high active transcription; providing a first dimer interaction domain that is linked with a domain configured for interaction with the promoter; providing a second dimer interaction domain that is linked with a domain configured for interaction with an RNA polymerase; and meeting a condition that is associated with an interaction between the first dimer interaction domain and the second dimer interaction domain, wherein the condition is met the interaction occurs and an activation complex is formed for activation of transcription.

In embodiments, the providing the promoter comprises producing the promoter by: mutagenizing one or more nucleotide sequences of a reference promoter to produce one or more mutagenic libraries each comprising a plurality of candidate promoters; cloning the plurality of candidate promoters of the one or more mutagenic libraries onto a plurality of inducible reporter constructs; transforming the plurality of inducible reporter constructs into one or more host cells; measuring a basal level of transcription of the plurality of inducible reporter constructs by the one or more host cells; and measuring an activated level of transcription of the plurality of inducible reporter constructs by the one or more host cells; wherein a difference between the activated level of transcription and the basal level of transcription suggests that the promoter has been produced.

In embodiments, providing the first and second dimer interaction domains comprises providing: a non-dimerized EL222 transcription factor that comprises a first homodimer interaction domain and a second homodimer interaction domain as the first and second dimer interaction domains, respectively; or a CRISPRa complex that comprises a dCas9 polypeptide complexed, via a gRNA or a sgRNA, with a MCP polypeptide that is linked with a first heterodimer interaction domain, and a second heterodimer interaction domain that is linked with a SoxS domain, wherein the first heterodimer interaction domain is the first dimer interaction domain and the second heterodimer interaction domain is the second dimer interaction domain.

In embodiments, the condition comprises: irradiation of a matrix that comprises the non-dimerized EL222 transcription factor with a light that comprises a blue light element for homodimerization of the non-dimerized EL222 transcription factor, or contact of a matrix that comprises the CRISPRa complex with a small molecule for heterodimerization of the CRISPRa complex.

In embodiments, the first heterodimer interaction domain comprises a SYNZIP A domain and the second heterodimer interaction domain comprises a SYNZIP B domain or the first heterodimer interaction domain comprises a SYNZIP B domain and the second heterodimer interaction domain comprises a SYNZIP A domain; the first heterodimer interaction domain comprises an ABI domain and the second heterodimer interaction domain comprises a PYL1 domain or the first heterodimer interaction domain comprises a PYL1 domain and the second heterodimer interaction domain comprises an ABI domain; or the first heterodimer interaction domain comprises a GAI domain and the second heterodimer interaction domain comprises a GID1 domain or the first heterodimer interaction domain comprises a GID1 domain and the second heterodimer interaction domain comprises a GAI domain.

In an aspect, the disclosure provides a method for producing a promoter comprising a minimal promoter sequence and an upstream element (UP-element) sequence that comprises a nucleotide base mutation for a modified promoter-RNA polymerase interaction to configure the promoter for a low basal transcription and a high active transcription, the method comprising: selecting a reference promoter; mutagenizing one or more nucleotide sequences of the reference promoter to produce one or more mutagenic libraries each comprising a plurality of candidate promoters; cloning the plurality of candidate promoters of the one or more mutagenic libraries onto a plurality of inducible reporter constructs; transforming the plurality of inducible reporter constructs into one or more host cells; measuring a basal level of transcription of the plurality of inducible reporter constructs by the one or more host cells; and measuring an activated level of transcription of the plurality of inducible reporter constructs by the one or more host cells.

In embodiments, a difference between the activated level of transcription and the basal level of transcription suggests that the promoter has either a higher dynamic range of transcription or a higher absolute range of transcription relative to corresponding metrics of the reference promoter.

In an aspect, the disclosure provides a method for producing a heterodimer CRISPRa system, the method comprising: providing a promoter comprising a minimal promoter sequence and an upstream element (UP-element) sequence that comprises a nucleotide base mutation for a modified promoter-RNA polymerase interaction to configure the promoter for a low basal transcription and a high active transcription; providing a first heterodimer domain linked to a MCP polypeptide; providing a second heterodimer domain linked to a SoxS domain; modifying a gRNA or a sgRNA; and testing the gRNA or the sgRNA for an ability of the gRNA or the sgRNA to activate transcription in combination with the promoter, the first heterodimer domain linked to the MCP polypeptide, and the second heterodimer domain linked to the SoxS domain; wherein the ability of the gRNA or the sgRNA to activate transcription is associated with a structure of the gRNA or the sgRNA and a target location within the promoter; wherein if the gRNA or the sgRNA sufficiently activates transcription in combination with the high-performance activatable promoter, the first heterodimer domain linked to the MCP polypeptide, and the second heterodimer domain linked to the SoxS domain, the heterodimer CRISPRa system is produced.

In embodiments, the method further comprises improving the structure of the gRNA or the sgRNA with the target location within the high-performance activatable promoter to improve the ability of the gRNA or the sgRNA to activate transcription.

In embodiments, the providing the first heterodimer domain linked to the MCP polypeptide comprises linking the first heterodimer domain to the MCP polypeptide and the providing the second heterodimer domain linked to the SoxS domain comprises linking the second heterodimer domain to the SoxS domain.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings.

FIG. 1 shows a high-level diagram of an example of conditionally-responsive CRISPRa for dynamic transcription activation, according to aspects of the disclosure.

FIG. 2A shows an example of an ABA-CRISPRa system for conditional transcription activation, according to aspects of the disclosure.

FIG. 2B shows an example of a GA-CRISPRa system for conditional transcription activation, according to aspects of the disclosure.

FIG. 2C shows an example of a SYNZIP system for conditional transcription activation, according to aspects of the disclosure.

FIG. 3 shows a bar graph comparing dynamic range of original and engineered promoters, showing the impact of novel CRISPRa systems on the dynamic range of expression, and how a design-driven library yields promoter variants with >100-fold improvement in dynamic range of expression, according to aspects of the disclosure.

FIG. 4A shows an example schematic of RNA polymerase (RNAP) interactions with the CRISPRa complex and target promoter, according to aspects of the disclosure. σ⁷⁰ affinity for the minimal promoter and α-CTD affinity for the UP-element determines RNAP recruitment to a promoter. When CRISPRa is targeted to a promoter with a complementary scRNA target site, the RNAP α-CTD domain is recruited by the SoxS transcriptional activator. RNAP-promoter and CRISPRa-promoter interactions can be modulated by modifying the DNA sequence of the different promoter parts.

FIG. 4B shows an example workflow for the assembly and characterization of libraries of activatable promoters, according to aspects of the disclosure. A library of RFP genes with varying promoters is generated through PCR (Example 1 Materials and Methods). The library is then co-transformed into E. coli with an aTc-inducible CRISPRa plasmid. Colonies are then seeded overnight in EZ-RDM. Overnight cultures are subsequently diluted into a fresh EZ-RDM and supplemented with appropriate concentrations of aTc. For each promoter variant in the libraries, basal and activated RFP levels were measured with 0 nM aTc and 200 nM aTc, respectively (Example 1 Materials and Methods).

FIG. 4C shows an example schematic of RNAP interaction with the minimal promoter and library design, according to aspects of the disclosure. σ⁰⁷⁰ recognizes specific positions in the extended −10 and −35 regions of the minimal promoter, which informed the design of Library 1. σ⁷⁰ binding is also influenced by the GC-content, the length, and the −¹⁵TGn⁻¹ motif of the intervening sequence, which informed the design of Library 2 (Example 1 Materials and Methods).

FIG. 4D shows an example minimal promoter effect on expression levels, according to aspects of the disclosure. Left: Schematic of inducible CRISPRa system and minimal promoter libraries of the J3 synthetic promoter. MCP-SoxS is expressed from the aTc-inducible pTet promoter. dCas9 and J306 scRNA are constitutively expressed. Right: Activated and basal RFP/OD₆₀₀ for the two minimal promoter libraries. Upper left curved dash line defines the Pareto front containing the best performing promoter variants (Example 1 Materials and Methods), for which no further improvements in basal or activated levels can be achieved without compromising the other. Lower right straight dash line defines promoter variants with equal activated and basal expression levels, indicating they are not activated by CRISPRa. The J23117 minimal promoter is included as a standard reference for CRISPRa efficiency. The J23119 minimal promoter is an example of a non-activatable promoter. A plasmid without RFP indicates the background fluorescence of the system.

FIG. 5A shows an example schematic of RNAP interactions with the UP-element and library design, according to aspects of the disclosure. α-CTD affinity for AT-rich UP-elements upstream of the minimal promoter helps recruit RNAP. Upon targeting with CRISPRa, UP-element RNAP recruitment contributions are largely replaced by SoxS-RNAP α-CTD interactions. UP-element libraries with increasing GC-content were designed to minimize α-CTD interactions (Example 1 Materials and Methods).

FIG. 5B shows an example of UP-element GC-content effect on expression levels through activated and basal RFP/OD₆₀₀ for the six UP-element libraries, according to aspects of the disclosure. Left: Increasing GC-content in the UP-element lowers the range of basal expression level, while maintaining the full range of activated expression levels. Dash line defines promoter variants with equal activated and basal expression levels, indicating they are not activated by CRISPRa. Right: Dash lines define the Pareto front containing the best performing promoter variants for each UP-element library (Example 1 Materials and Methods). Increasing the UP-element GC-content effectively shifts the Pareto front towards lower basal expression levels.

FIG. 5C shows scRNA target site composition effect on basal expression, according to aspects of the disclosure. Comparison of three scRNA libraries with increasing GC-content (Example 1 Materials and Methods). Basal expression levels are normalized to the standard J3 promoter basal expression level. Lines within boxes indicate the median expression level of each distribution. The interquartile range (IQR) is calculated as the difference between the upper and lower quartiles and measures the spread of the distribution.

FIG. 5D shows sequential construction of activatable promoters, according to aspects of the disclosure. Left: Activatable promoters were constructed by sequential library mutagenesis screens starting from the J3 promoter with a GC-rich scRNA target site. Three Pareto optimal UP-elements were selected after promoter mutagenesis with a GC-rich UP-element library (1). The minimal promoter of the three previously selected variants was then mutagenized (2), and three Pareto optimal variants were again selected. Right: Basal and activated expression levels for all mutagenesis variants normalized to the standard J3 promoter expression levels (dot positioned above text “original promoter”). Lower points represent variants from the UP-element mutagenesis (1), while upper points represent variants from the minimal promoter mutagenesis (2). Larger circles indicate selected variants from each screen, and solid lines depict the Pareto optimal fronts. Each sequential mutagenesis led to variants with both lower basal and higher activated expression levels.

FIG. 6A shows an example schematic of orthogonal CRISPRa/i nodes for use in cell-free circuits, according to aspects of the disclosure. Internal nodes contain an orthogonal scRNA target site and express orthogonal scRNAs. Output nodes contain orthogonal scRNA target sites and express mRFP1. All nodes contain the same UP-element and minimal promoter.

FIG. 6B shows an example high-throughput characterization of scRNA components in CFS, according to aspects of the disclosure. Left: Plasmids encoding each CRISPRa component are mixed using an acoustic liquid handling robot and expressed in CFS. Right: scRNA-dose response curves for each node are generated by titrating the amount of scRNA plasmid from 0.5 pM to 5 nM. Values represent the mean±standard deviation of three technical replicates.

FIG. 6C shows an example comparison of assembly strategies for building a four-layer CRISPRa cascade, according to aspects of the disclosure. Left: Internal node concentrations either decreased from 200 pM to 32 pM as depth increased, were held constant at 200 pM, or increased from 200 pM to 1.25 nM as depth increased. A fourth assembly method was tested in which internal node concentrations were 40, 200, and 170 pM, based on individual scRNA-dose response characteristics. A negative control was included in which the high-performing promoter of the second internal node was replaced with the leaky J2 promoter. Input and output node concentrations were held constant across all strategies at 0 pM or 15 pM and 10 nM, respectively. Center: Cascade output RFP expression for each assembly strategy with scRNA input (lower bars in bar pairs) and without (upper bars in bar pairs), relative to RFP basal expression. Values represent the mean±standard deviation of three technical replicates. Right: Change in time to maximum expression rate (Δtₘₐₓ) for each assembly strategy. Δtₘₐₓ is calculated by finding the difference in time to reach maximum RFP production rate between the with and without input conditions (Example 1 Materials and Methods). Values represent the mean±standard deviation of three technical replicates.

FIG. 6D shows an example of rapid fold change optimization of a four-layer CRISPRa cascade, according to aspects of the disclosure. Left: The first and third internal nodes of the cascade were varied between 40 and 160 pM, and 85 and 340 pM, respectively. The input node, second internal node, and output node were held constant at 0 pM or 15 pM, 0.2 nM, and 10 nM, respectively. Right: Fold change between with and without scRNA input for each CRISPRa cascade. Values represent the mean±standard deviation of three technical replicates.

FIG. 6E shows an example of signal propagation through deep CRISPRa/i circuits, according to aspects of the disclosure. Left: Schematic of CRISPRa cascades with increasing depth. Input and output node concentrations were held constant across all cascades at 0 or 15 pM and 10 nM, respectively. All node concentrations are tabulated in Table 5. Right: Propagation efficiency and signal delay are shown as a function of circuit depth. Propagation efficiency for each cascade is calculated as the ratio between the fold change of the output RFP compared to the fold change of the input layer (Example 1 Materials and Methods). Signal delay is calculated as the difference in time to reach the maximum fold-activation of the cascade between the cascade output and input layer (Example 1 Materials and Methods). Values represent the mean±standard deviation of three technical replicates.

FIG. 6F shows an example of construction of wide CRISPRa/i circuits, according to aspects of the disclosure. Left: Schematic of CRISPRa cascades with increasing width. Input and output node concentrations were held constant across all cascades at 0 pM or 15 pM and 10 nM, respectively. Right: The concentration of each internal node was held at 0.2 nM as circuit width increased (lower data points), or the internal node concentration was scaled down proportionally to the width of the circuit (upper data points), such that each internal node concentration is 0.2/n nM, where n is the number of parallel cascades. Fold-activation is given relative to a single CRISPRa cascade (Example 1 Materials and Methods). Values represent the mean±standard deviation of three technical replicates.

FIG. 7A shows an example schematic of EL222 light-responsive promoter system and library design, according to aspects of the disclosure. EL222 transcription factor dimerizes in response to 470 nm light and binds a specific sequence upstream of the minimal promoter. EL222 then recruits RNAP through interactions with the α-CTD domain. Minimal promoter library design is based on the original lux promoter and previous minimal promoter libraries (Example 1 Materials and Methods).

FIG. 7B shows an example characterization of light responsive promoters in E. coli, according to aspects of the disclosure. Left: Schematic of blue light promoter screening (Example 1 Materials and Methods). EL222 protein and promoter library are expressed from a single plasmid. Assembly and screening are carried out as previously described. Basal and activated expression levels are measured from cultures not exposed or continuously exposed to blue light, respectively. Right: Basal expression and dynamic range of blue light promoter variants. Lower dash line defines promoter variants with equal activated and basal expression levels, indicating they are not activated by EL222. The J23119 minimal promoter and J23113 are examples of non-activatable promoters. Variants with improved performance (e.g., P1, P2, P3, P4) compared to the original luxI promoter were selected for use in CFS.

FIG. 7C shows an example of light-responsive CRISPRa in CFS, according to aspects of the disclosure. Left: Schematic of EL222 scRNA expression and downstream CRISPRa is achieved by expressing a scRNA from an engineered blue light promoter. Right: Titration of blue light inducible scRNA plasmid concentration to maximize the fold change between blue light dependent CRISPRa and CRISPRa due to scRNA leak in the dark; light (upper data points) and dark (lower data points). Values represent the mean±standard deviation of three technical replicates.

FIG. 7D shows an example of improvement of blue light CRISPRa dynamic range through the construction of a positive feedback circuit, according to aspects of the disclosure. Left: Schematic of a blue light responsive CRISPRa cascade with positive feedback (PFB). PFB is achieved by including a downstream node that expresses a scRNA targeting an upstream node. Right: Blue light dependent CRISPRa and CRISPRa due to scRNA leak in the dark; light (right bars in bar pairs) and dark (left bars in bar pairs). The amount of positive feedback was tuned by adjusting the concentration of the PFB node. Values represent the mean±standard deviation of three technical replicates.

FIG. 8A shows an example schematic of different conditional CRISPRa systems, according to aspects of the disclosure. MCP-SoxS fusion is split and the two proteins are instead fused to one end of a heterodimerization domain. The heterodimerization domains used to build conditional CRISPRa systems are the SYNZIP5/SYNZIP6 pair, the abscisic acid (ABA)-responsive ABI/PYL1 domain, and the gibberellic acid (GA)-responsive GAI/GID1 domain.

FIG. 8B shows example distance requirements of conditional CRISPRa, according to aspects of the disclosure. Left: An engineered promoter containing densely packed scRNA target sites and single base pair additions allows for CRISPRa targeting with single base pair resolution between −81 and −111 bp from the TSS. Phase shifts are introduced at the 5′ end of the promoter UP-element. Right: Testing SYNZIP-CRISPRa with single base pair resolution between −81 and −91 bp from the TSS. SYNZIP-CRISPRa components are expressed at 5 nM. Fold change is calculated relative to an off-target scRNA for each promoter variant.

FIG. 8C shows an example of tuning conditional CRISPRa response through titration of small molecule concentration, according to aspects of the disclosure. For ABA- and GA-CRISPRa, the corresponding small molecule was titrated between 0 and 10 or 0 and 10³ μM respectively to find the optimal concentration. ABA- and GA-CRISPRa components are expressed at 5 nM.

FIG. 8D shows an example of improving conditional CRISPRa response by optimizing component stoichiometries, according to aspects of the disclosure. The concentration of the plasmids expressing the MCP and SoxS components for each dimerization system were varied between 1 and 25 nM and tested combinatorially to find the best ratio of the two heterodimerization proteins. ABA is added at 10 μM and GA is added at 10³ μM. Fold change is given relative to a reaction with no MCP and SoxS plasmids added.

FIG. 9A shows an example conditional CRISPRa response to non-cognate ligands, according to aspects of the disclosure. The orthogonality of the small molecule-responsive conditional CRISPRa systems was tested by adding either the corresponding or non-corresponding small molecule to cell-free reactions containing the machinery for ABA- or GA-CRISPRa. The components for both ABA- and GA-CRISPRa are added at their respective optimal screened concentrations; ABA-CRISPRa (left bars of bar pairs) and GA-CRISPRa (right bars of bar pairs). ABA is added at 10 μM and GA is added at 10³ μM. Values represent the mean±standard deviation of three technical replicates.

FIG. 9B shows an example assembly of conditional CRISPRa circuits, according to aspects of the disclosure. For both circuits, the components for both ABA- and GA-CRISPRa are added at their respective optimal screened concentrations. ABA is added at 10 μM and GA is added at 103 μM. (Left) The AND gate was constructed by adding the components for both ABA- and GA-CRISPRa in a cell-free reaction such that only the addition of both ligands would result in significant activation. Fold-change is given relative to a reaction with neither ligand added. (Right) The CRISPRa cascade was assembled by using ABA-CRISPRa to activate expression of both the first and second node in an activation cascade. The first node was added at either 0.05 nM or 0 nM, and the internal and output nodes were added at 10 nM. Values represent the mean±standard deviation of three technical replicates.

FIG. 9C shows an example simulation analysis of a two-input CRISPRa/i circuit using SYNZIP-CRISPRa and blue light CRISPRI, according to aspects of the disclosure. The simulation sampled different amounts of CRISPRa and CRISPRi input to analyze whether the two systems could be integrated to modulate the production rate of a desired gene target (Example 1 Materials and Methods).

FIG. 9D shows an example of how SYNZIP-CRISPRa and blue light CRISPRi were integrated to construct a tunable pulse generator, according to aspects of the disclosure. The amount of CRISPRa was tuned by adding either 0.2 nM or 1 nM of constitutively expressed scRNA plasmid to the CFS reaction. The sgRNA targeting RFP for CRISPRi was driven from the blue light responsive engineered EL222 promoter. The amount of CRISPRi was tuned by adjusting the time of blue light exposure between 45 and 135 min. RFP production rates are plotted as a function of CRISPRa and CRISPRi inputs (Example 1 Materials and Methods); vertical positions of scRNA DNA concentrations within the figure legend correspond with vertical positions of data points in the graph. Values represent the mean±standard deviation of three technical replicates.

FIG. 10A shows an example of the minimal promoter effect on fold-change, according to aspects of the disclosure. Basal RFP/OD₆₀₀ and fold change for the two minimal promoter libraries (nMP1=89, nMP2=84). The J23117 minimal promoter (three black dots at about 10² Basal RFP/OD₆₀₀ and 60-Fold Change) is included as a standard reference for CRISPRa efficiency. The J23119 minimal promoter (three black dots at about 10⁴ Basal RFP/OD₆₀₀ and 0-Fold Change) is an example of a non-activatable promoter due to high basal expression levels.

FIG. 10B shows an example of UP-element libraries RFP distributions, according to aspects of the disclosure. Histograms and probability density functions for basal expression (left; gray) and activated expression (right; gray and black) for six UP-element libraries with increasing GC-content. Median fold change is calculated as the ratio of median activation and media basal expression levels.

FIG. 10C shows an example of UP-element libraries fold-change distributions, according to aspects of the disclosure. Histograms and probability density functions for fold change for six UP-element libraries with increasing GC-content.

FIG. 10D shows an example of CRISPRa activity validation of high GC-content scRNAs, according to aspects of the disclosure. Fold change upon aTc induction and basal expression. scRNA target site sequences were initially selected based on low expression leak in E. coli and the corresponding scRNAs were constructed for use in CFS. Selected scRNAs were benchmarked against the standard J306 scRNA (gray).

FIG. 10E shows an example of combinatorial construction of activatable promoters, according to aspects of the disclosure. In addition to the J3.J23117 benchmark, three high performing variants each from the UP-element and minimal promoter libraries were tested in a combinatorial manner for a total of 16 UP-element/minimal promoter combinations screened in E. coli. The basal and activation expression of the 16 promoters were quantified with the same scRNA (bottom). Activation ratio is calculated by dividing the activated RFP expression from the inducible CRISPRa system by the basal RFP expression from each promoter (top). Values represent the mean±standard deviation of three technical replicates.

FIG. 10F shows an example of activatable promoter characterization in CFS, according to aspects of the disclosure. Characterization of selected promoter variants in CFS. CRISPRa-mediated RFP expression levels (gray, 0.4 nM scRNA DNA) and RFP basal expression levels (black, 0 nM scRNA DNA). Reactions contain 10 nM of RFP plasmid. Values represent the mean±standard deviation of three technical replicates.

FIG. 10G shows example scRNA dose-response characterizations, according to aspects of the disclosure. scRNA dose-response curves are shown for orthogonal promoter-scRNA pairs in CFS. The scRNA-dose response curve is characterized through titrating the amount of scRNA DNA added to the CFS reaction. Reactions contain 10 nM of RFP plasmid. Black line indicates a logistic fit to the data. Values represent the mean±standard deviation of three technical replicates.

FIG. 10H shows an example of titration of middle node in two-layer activation cascade, according to aspects of the disclosure. Two-layer activation cascade with high-performing components to identify the best performing internal node concentration. Left: Circuit schematic for measuring output RFP and fold change as a function of input scRNA. Right: Cascade RFP output with scRNA input (15 pM, gray) and without (0 pM, black). Output node concentration is held constant at 10 nM. Values represent the mean±standard deviation of three technical replicates.

FIG. 10I shows an example of signal propagation in a two-layer activation cascade, according to aspects of the disclosure. gRNA competition impact on circuit function. Left: Circuit schematic for measuring output fold change as a function of input scRNA for both CRISPRa (black) and CRISPRa cascade (gray). Internal node concentration and output node concentration are held constant at 0.2 nM and 10 nM, respectively. Right: Input scRNA plasmid concentration was titrated between 1 pM and 2 nM. Black dashed line indicates saturation of CRISPRa complexes with input scRNA. Values represent the mean±standard deviation of three technical replicates.

FIG. 10J shows an example time course for four-layer activation cascade assembly strategies, according to aspects of the disclosure. Comparison of the dynamics of four-layer CRISPRa cascade assemblies. Left: Internal node concentrations either decreased from 200 pM to 32 pM as depth increased, were held constant at 200 pM, or increased from 200 pM to 1.25 nM as depth increased. A fourth assembly method was tested in which internal node concentrations were 40, 200, and 170 pM, based on individual scRNA-dose response characteristics. A fifth cascade was included in which the high-performing promoter of the second internal node was replaced with the leaky J2 promoter. Input and output node concentrations were held constant across all strategies at 0 or 15 pM and 10 nM, respectively. Right: Output RFP expression for each assembly strategy with scRNA input (gray) and without (black). Values represent the mean±standard deviation of three technical replicates. Time to maximum expression rate (tₘₐₓ) for each assembly strategy is calculated by finding the time to reach maximum RFP production rate between (Example 1 Materials and Methods).

FIG. 10K shows an example four-layer activation cascade basal and activated RFP expression, according to aspects of the disclosure. Left: Basal expression levels for cascades titrating the first and third layers between 40 and 160 pM, and 85 and 340 pM, respectively. Right: Activated expression levels for the same cascades. The input node, second internal node, and output node were held constant at 0 or 15 pM, 0.2 nM, and 10 nM, respectively. Values are not background subtracted. Values represent the mean±standard deviation of three technical replicates.

FIG. 10L shows example signal propagation and signal delay model accuracy, according to aspects of the disclosure. Comparisons of measured and predicted signal propagation (left) and signal delay (right) for activation cascades of different depths. Signal propagation is calculated by dividing the fold-activation of the cascade output by the fold-activation from the input layer. (Example 1 Materials and Methods). Signal delay is calculated as the difference between the cascade output and input layer in time to reach the maximum fold-activation (Example 1 Materials and Methods). Both measures are presented as the mean±standard deviation of three technical replicates. The fold-change from the individual promoters' dose-response curves (FIG. 10G) are used to iteratively predict the delay and signal propagation at the next layer (Example 1 Materials and Methods). Black line represents the mean±standard deviation of linear regression model measuring goodness-of-fit between model predictions and experimental data.

FIG. 10M shows an example of wide circuit basal and activated RFP expression, according to aspects of the disclosure. Up to four parallel three-layer cascades were constructed. Left: The concentration of each internal node was held at 0.2 nM as circuit width increased. Right: The internal node concentration is scaled down proportionally to the width of the circuit, such that each internal node concentration is 0.2/n nM, where n is the number of parallel cascades.

FIG. 10N shows an example of blue light promoter characterization in CFS, according to aspects of the disclosure. Characterization of selected promoter variants in CFS. Reactions contain 8 nM and 10 nM of EL222 and RFP plasmids respectively. EL222-mediated RFP expression levels (gray) and RFP basal expression levels (black). Values represent the mean #standard deviation of three technical replicates.

FIG. 10O shows an example of blue light CRISPRi, according to aspects of the disclosure. Titration of blue light inducible sgRNA plasmid concentration to maximize the fold repression between blue light dependent CRISPRi (gray; light) and CRISPRi due to sgRNA leak in the dark (black; dark). Reactions contain 8 nM and 1 nM of EL222 and RFP plasmids respectively. Values represent the mean±standard deviation of three technical replicates.

FIG. 10P shows an example of EL222 titration in blue light CRISPRa, according to aspects of the disclosure. Titration of EL222 plasmid concentration to maximize the fold change between blue light dependent CRISPRa (gray; light) and CRISPRa due to scRNA leak in the dark (black; dark). Reactions contain 10 nM RFP plasmid. Values represent the mean±standard deviation of three technical replicates.

FIG. 10Q shows an example of fusion orientation preference for SYNZIP and ABI/PYL1, according to aspects of the disclosure. Top Left, Top Right: MCP and SoxS fusion orientations were tested for the SYNZIP-CRISPRa system in E. coli using the J306 spacer at −81 bp from the TSS. The MCP test was done using SoxS-SYNZIP and the SoxS test was done using MCP-SYNZIP. Bottom: SoxS fusions were tested for the abscisic acid (ABA) CRISPRa system using MCP-ABI. The ABA constructs were tested in CFS using the R206 spacer at −81 bp from the TSS. ABA components were expressed at 5 nM. Off-target represents reactions containing a scRNA with no cognate target. Values represent the mean±standard deviation of three technical replicates.

FIG. 10R shows an example of dependence of SYNZIP-CRISPRa on distance to TSS, according to aspects of the disclosure. SYNZIP-CRISPRa and CRISPRa were tested at various target sites with increasing distance from the TSS in 10 bp intervals using a CRISPRa promoter with densely packed scRNA target sites. Plasmids expressing SYNZIP components are added at 5 nM each. Values represent the mean±standard deviation of three technical replicates.

FIG. 10S shows example improvements in SYNZIP-CRISPRa from engineered promoters, according to aspects of the disclosure. Comparison of SYNZIP-CRISPRa scRNA-dependent fold change with the previous synthetic promoter used to survey target sites and an engineered high dynamic range promoter. Off-target represents reactions containing a scRNA with no cognate target. In each reaction, the concentration of reporter DNA was 10 nM. SYNZIP components are added at 5 nM each. Reactions are background subtracted from a cell-free reaction containing no DNA. Values represent the mean±standard deviation of three technical replicates.

FIG. 10T shows an example of conditional CRISPRa scRNA dose-response, according to aspects of the disclosure. scRNA-dose response curves were collected for conditional CRISPRa systems. scRNA concentrations were titrated between 10⁻³ and 10¹ nM. Fold activation was calculated relative to the no scRNA condition. For all conditions, ABA is added at 10 μM and GA is added at 10³ μM. SYNZIP-CRISPRa components were both added at 5 nM, MCP-ABI and SoxS-PYL1 were added at 5 and 10 nM respectively, and GA-CRISPRa components were both added at 10 nM. Colored lines indicate a logistic fit to the data. Values represent the mean±standard deviation of three technical replicates.

FIG. 10U shows an example comparison of CRISPRa promoters to pTet, according to aspects of the disclosure. Comparison of RFP expression levels of different CRISPRa promoters (gray) to the pTet system (green). In both systems, RFP plasmid copy number and RBS remained constant. Basal expression level (“−”) is measured with off-target scRNAs for the CRISPRa promoters, and 0 nM aTc for pTet. Activated expression level (“+”) is measured with on-target scRNAs for the CRISPRa promoters, and 200 nM aTc for pTet. The J23117 and pTet values represent the mean±standard deviation of three technical replicates, whereas HP1-3 values correspond to the individual variants from the sequential screen (FIG. 5D).

FIG. 10V shows example three-layer activation cascades in E. coli, according to aspects of the disclosure. Three-layer activation cascades in E. coli with the input controlled by pTet and internal nodes expressed from different copy number plasmids. Top: Schematic of plasmids used for the different nodes. Input and output layers were kept constant across conditions, and the copy number of the plasmids encoding the two internal layers was varied between ColEI (high copy) and CloDF13 (low copy). Top graph: GFP output for the different activation cascades at 0 nM aTc. Leak is minimized in the system when the third scRNA is expressed from a low copy number plasmid. Leak is also lowered when the second scRNA is present due to gRNA competition with the third scRNA. Expressing the second scRNA from a high copy number resulted in higher fold-activation. Bottom graph: When induced with 100 nM aTc, cascade output is reduced, possibly due to competition of the first scRNA with downstream scRNAs. Values represent the mean #standard deviation of three technical replicates.

FIG. 11A shows an example graph illustrating how extended CRISPRa is sensitive to the orientation of activator protein fusions; on target (left bars of bar pairs) and off target (right bars of bar pairs), according to aspects of the disclosure.

FIG. 11B shows an example graph of results from using CRISPRa to characterize promoter variants produced by mutagenesis, according to aspects of the disclosure.

FIG. 11C shows an example graph of results from using CRISPRa to characterize promoter variants including random sequences, high G/C content, and very high G/C content, according to aspects of the disclosure.

FIG. 11D shows examples of UP-element libraries' RFP distributions, according to aspects of the disclosure. Histograms and probability density functions for basal expression (left data points) and activated expression (right data points) for six UP-element libraries with increasing GC-content. Median fold change is calculated as the ratio of median activation and media basal expression levels.

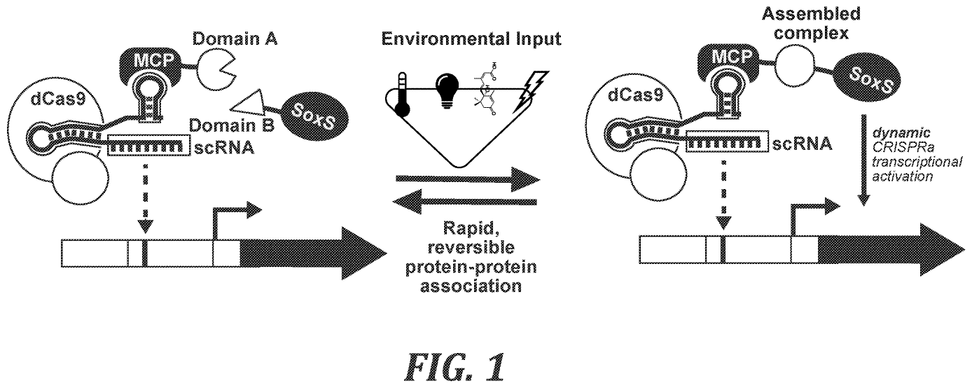

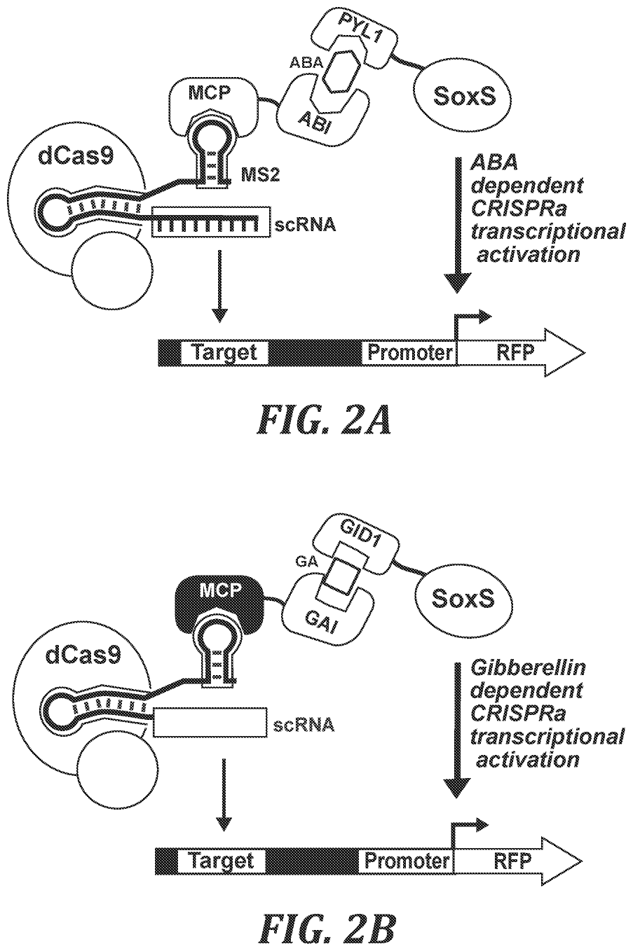

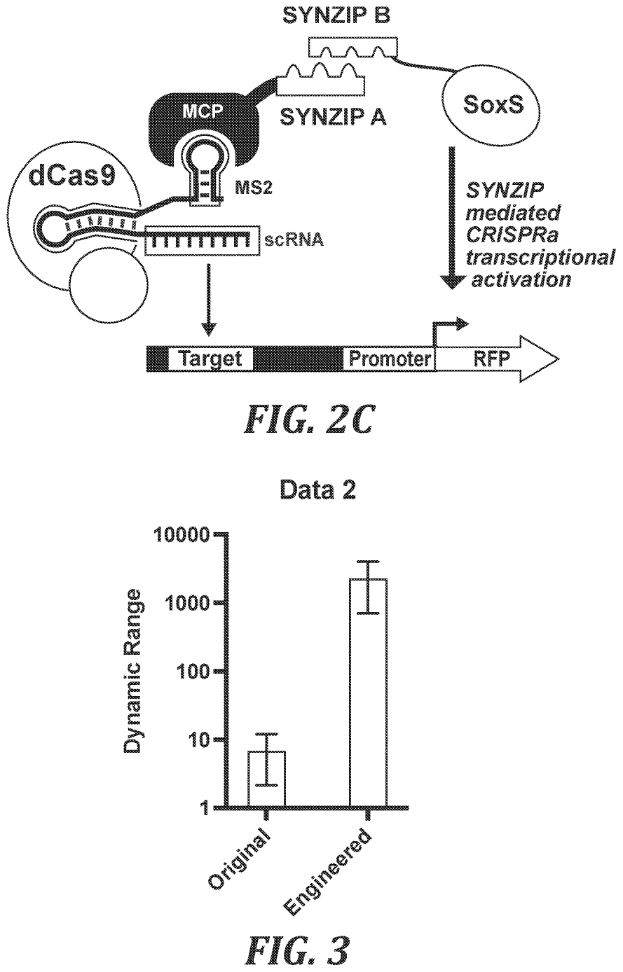

### DETAILED DESCRIPTION

The disclosure provides high-performance activatable promoters, and CRISPRa/i nodes, for use with CRISPR-based synthetic GRNs. The promoters, CRISPRa/i nodes, and GRNs, and components thereof, can be used in a variety of applications, including but not limited to metabolic engineering applications, information processing applications, biosensor applications, biosynthesis applications, and others. The promoters include nucleotide base mutations for altered interactions with RNA polymerase, and are engineered to have a lower basal level of transcription, a higher activated level of transcription, or both, compared to previous iterations of promoters for cells and cell-free systems. In this manner, the synthetic GRNs can better sense and respond to environmental inputs. The disclosure also provides methods for engineering the disclosed and other high-performance activatable promoters with mutagenesis.

### High-Performance Activatable Promoters

High-performance activatable promoters are disclosed and configured for low basal transcription and high activated transcription in a cell or a cell-free system (CFS). In general, the promoters include a minimal promoter sequence and an upstream element (UP-element) sequence that comprises a nucleotide base mutation for a modified promoter-RNA polymerase interaction. In this manner, the promoter is configured for a low basal transcription and a high active transcription. The promoters comprise sequences that are mutated and can be at least approximately Pareto optimal for low basal transcription and high active transcription, and an improved or optimal balance is achieved between background (basal expression) and signal (activated expression).

While E. coli promoters and molecular components are used as examples herein, other promoters and molecular components, e.g., from other bacteria or other organisms, can be implemented without departing from the scope and spirit of the disclosure. E. coli promoters contain a minimal promoter sequence positioned 5′ of the transcription start site (TSS) and an UP-element sequence positioned 5′ of the minimal promoter sequence. The UP-element facilitates recruitment of RNA polymerase to the minimal promoter sequence for facilitation of transcription.

In embodiments, the UP-element sequence of a disclosed promoter comprises a higher GC content relative to GC contents of corresponding reference sequences, such as naturally occurring UP-element sequences.

As used herein, “GC content” refers to an amount or percentage of nucleotide bases within a nucleotide sequence that are either guanine (G) or cytosine (C). “AT content” refers to an amount or percentage of nucleotide bases within a nucleotide sequence that are either adenine (A) or thymine (T). Given a particular sequence, as the GC content generally increases, the AT content generally decreases, as would be understood by a person having ordinary skill in the art. In addition, while standard DNA bases are disclosed herein, it is envisaged that modified or alternate DNA bases can be implemented in embodiments without departing from the scope and spirit of the disclosure. For example, “GC content” can refer to the content of guanine and cytosine, modified guanine, modified cytosine, modified guanine and modified cytosine, and the like, in a nucleotide sequence. Likewise, “AC content” can refer to the content of adenine and thymine, modified adenine, modified thymine, modified adenine and modified thymine, and the like, in a nucleotide sequence.

In embodiments, the UP-element sequence comprises between 50% and 100% GC content, or is more inclusive of GC content relative to a reference minimal promoter sequence.

As used herein, “more inclusive of GC content” refers to one or more nucleotide base additions, deletions, insertions, or mutations within a nucleotide sequence, such that the resultant mutated nucleotide sequence contains a higher GC content. Such a mutation can include, for example, mutation for decreasing AT content, mutation for increasing GC content, or both.

As used herein, GC content percent is calculated by dividing the number of bases in a nucleotide sequence that are either G or C (or modified or alternate G or C) by the total number of bases in the nucleotide sequence for a ratio, and multiplying the ratio by 100%.

As used herein, ranges given for amounts and percentages are inclusive of the amounts and percentages that define the range, unless something else is specifically stated. In addition, the disclosure of an amount or percentage includes the disclosed amount or percentage, as well as amounts or percentages that fall within the range of −10% and +10% of the amount or percentage given, unless something else is specifically stated.

In embodiments, the UP-element sequence comprises at least 80% identity with a sequence selected from SEQ ID NOs: 33-49, and the minimal promoter sequence comprises at least 80% identity with a sequence selected from SEQ ID NOs: 50-66.

As used herein, “percent identity,” for example, “80% identity,” refers to the number of nucleotide bases in a given sequence that are the same as corresponding nucleotide bases in a reference sequence, when the given sequence and the reference sequence are aligned, expressed as a percentage. For example, a given sequence of ATGTGCATCG has 80% identity to a reference sequence of ATGTGCGCCG (8 bases are the same, 2 bases differ, 10 bases total; 8/10=80%).

In embodiments, the UP-element sequence comprises at least 81%, 82%, 83%, 84%, 85%, 86%, 87%, 88%, 89%, 90%, 91%, 92%, 93%, 94%, 95%, 96%, 97%, 98%, or 99% identity with a sequence selected from SEQ ID NOs: 33-49; and the minimal promoter sequence comprises at least 81%, 82%, 83%, 84%, 85%, 86%, 87%, 88%, 89%, 90%, 91%, 92%, 93%, 94%, 95%, 96%, 97%, 98%, or 99% identity with a sequence selected from SEQ ID NOs: 50-66. In embodiments of the promoter, the UP-element sequence comprises 100% identity with a sequence selected from SEQ ID NOs: 33-49; and the minimal promoter sequence comprises 100% identity with a sequence selected from SEQ ID NOs: 50-66.

In embodiments, the promoter further comprises a scaffold RNA (scRNA) target sequence configured to bind a guide RNA (gRNA) or single guide RNA (sgRNA) for localization of a CRISPRa complex to the promoter. The CRISPRa complex comprises a deactivated Cas9 (dCas9) linked with a MCP polypeptide and a first heterodimer interaction domain. As used herein, “scRNA target” refers to the scRNA target sequence of the promoter, positioned 5′ of the UP-element, that localizes the CRISPRa complex to the promoter.

In embodiments, the scRNA target sequence comprises a higher GC content relative to a GC content of a corresponding reference sequence.

In embodiments, the scRNA target sequence comprises at least 80%, 81%, 82%, 83%, 84%, 85%, 86%, 87%, 88%, 89%, 90%, 91%, 92%, 93%, 94%, 95%, 96%, 97%, 98%, or 99% identity with a sequence selected from SEQ ID NOs: 20-32. In embodiments of the promoter, the scRNA target sequence comprises 100% identity with a sequence selected from SEQ ID NOs: 20-32.

In embodiments, the promoter comprises at least 80%, 81%, 82%, 83%, 84%, 85%, 86%, 87%, 88%, 89%, 90%, 91%, 92%, 93%, 94%, 95%, 96%, 97%, 98%, or 99% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66. In embodiments, the promoter comprises 100% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

In embodiments, the promoter comprises a scRNA target sequence, an UP-element, and a minimal promoter sequence according to the disclosure that are connected, in tandem, in a 5′ position relative to the TSS. In embodiments, there are no intervening nucleotide bases between the scRNA target sequence and the UP-element, or between the UP-element and the minimal promoter sequence.

### CRISPRa/i Nodes

The disclosure also provides high-performance activatable CRISPRa/i nodes that include one or more promoters of the disclosure, that help configure the CRISPRa/i nodes for a low basal transcription and a high active transcription in a cell or a CFS. A CRISPRa/i node comprises a promoter comprising an UP-element sequence and a minimal promoter sequence. In embodiments, sequences of the promoter are sub-Pareto optimal or at least approximately Pareto optimal for low basal transcription and high active transcription, and a dCas9 polypeptide is complexed, via a gRNA or a sgRNA, with a MCP polypeptide that is linked with a first heterodimer interaction domain. A second heterodimer interaction domain that is linked with a SoxS domain. The gRNA or the sgRNA is configured to interact with the promoter to localize the first heterodimer interaction domain to the promoter, and the first heterodimer interaction domain is configured to conditionally interact with the second heterodimer interaction domain to localize the SoxS domain to the promoter.

As used herein, “CRISPRa/i node” refers to a set of CRISPR components that is capable of functioning as a node in a network of one or more such nodes. The “CRISPR” portion of “CRISPRa/i” refers to the node being comprised of CRISPR components, and the “a/i” portion of “CRISPRa/i” refers to the node being capable of CRISPR activation (“a”), CRISPR interference (“i”), or both.

In embodiments, a condition is associated with an interaction between the first heterodimer interaction domain and the second heterodimer interaction domain. If the condition is met, the interaction occurs, and an activation complex is formed for activation of transcription via the SoxS domain, which interacts with RNAP. In embodiments, the condition comprises irradiation of a matrix that comprises the CRISPRa/i node with a light that comprises a blue light element, contact of the matrix that comprises the CRISPRa/i node with a small molecule, or both. In embodiments, the CRISPRa/i node can be implemented for detection of light or blue light, presence of a small molecule, or both. In embodiments, the condition comprises irradiation of the matrix with the light that comprises the blue light element and the method is performed using a EL222 light-responsive promoter system.

While any of various conditional protein-protein interactions can be used for the heterodimer domain interaction, in example embodiments of the disclosure, the first heterodimer interaction domain comprises a SYNZIP A domain and the second heterodimer interaction domain comprises a SYNZIP B domain or vice versa; the first heterodimer interaction domain comprises an ABI domain and the second heterodimer interaction domain comprises a PYL1 domain or vice versa; or the first heterodimer interaction domain comprises a GAI domain and the second heterodimer interaction domain comprises a GID1 domain or vice versa. In embodiments, the CRISPRa/i node includes or implements any promoter or promoter element (e.g., scRNA target sequence, UP-element sequence, minimal promoter sequence), as described herein.

### Synthetic Gene Regulatory Networks (Grns)

The disclosure also provides synthetic gene regulatory networks (GRNs) that include one or more CRISPRa/i nodes. In example embodiments, a GRN comprises a first CRISPRa/i node and a second CRISPRa/i node, and the first CRISPRa/i node comprises a first promoter and the second CRISPRa/i node comprises a second promoter. During operation of the GRN, the first CRISPRa/i node senses an input to the network (e.g., light, small molecule) and the second CRISPRa/i node propagates a signal resulting from the sensed input, reports the sensed input in a manner that is detectable by an observer or detector, or both. Since small amounts of the input can be relevant to the application for which the GRN is implemented (e.g., biomedical monitoring, medical diagnostics, medical implants, environmental monitoring, and the like), amplification of the input signal by the network increases the sensitivity of the GRN and decreases the likelihood of false negatives.

In embodiments, a first transcription product results from activation of transcription of the first CRISPRa/i node and is an input to the second CRISPRa/i node, and a second transcription product results from activation of transcription of the second CRISPRa/i node and is an output of the second CRISPRa/i node, the synthetic GRN, or both. For example, the first CRISPRa/i node can sense an analyte and activate transcription from the first promoter to transcribe a gRNA or an sgRNA which in turn localizes the CRISPR complex to the second promoter for transcription of a detectable product, such as a fluorescent protein, for example.

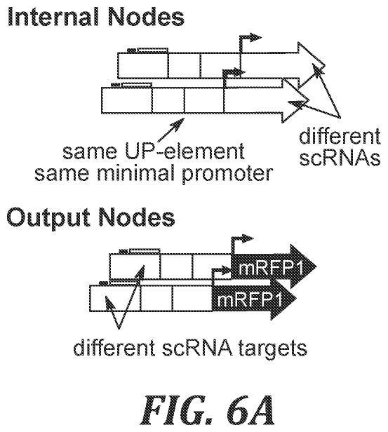

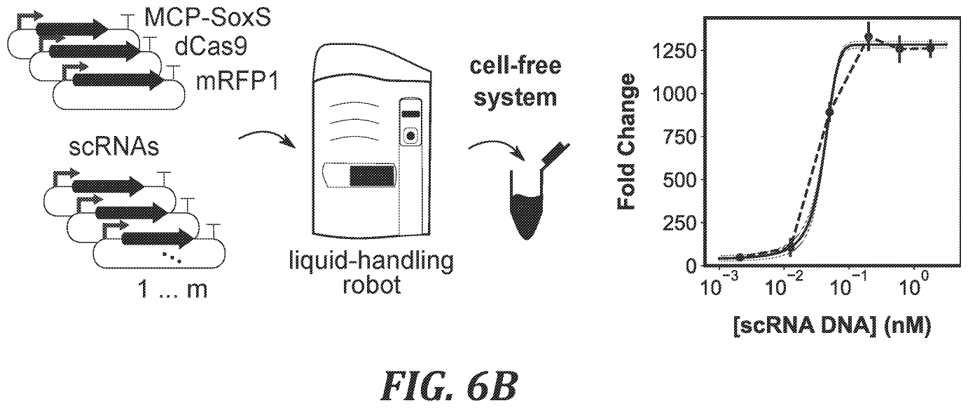

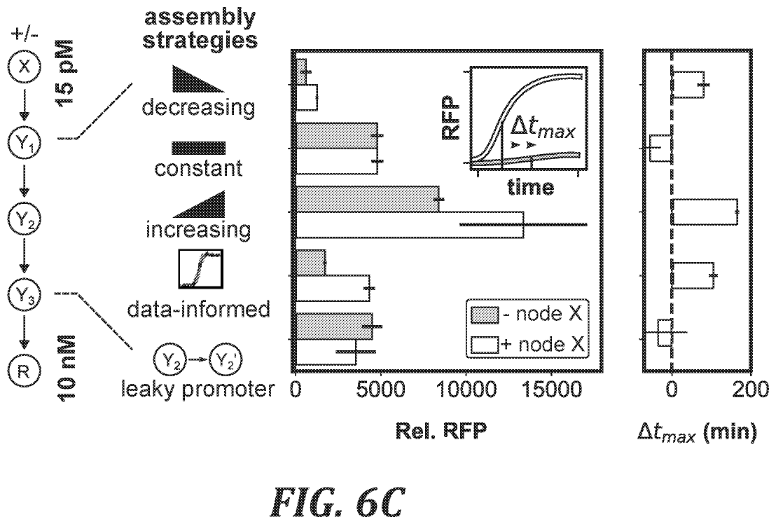

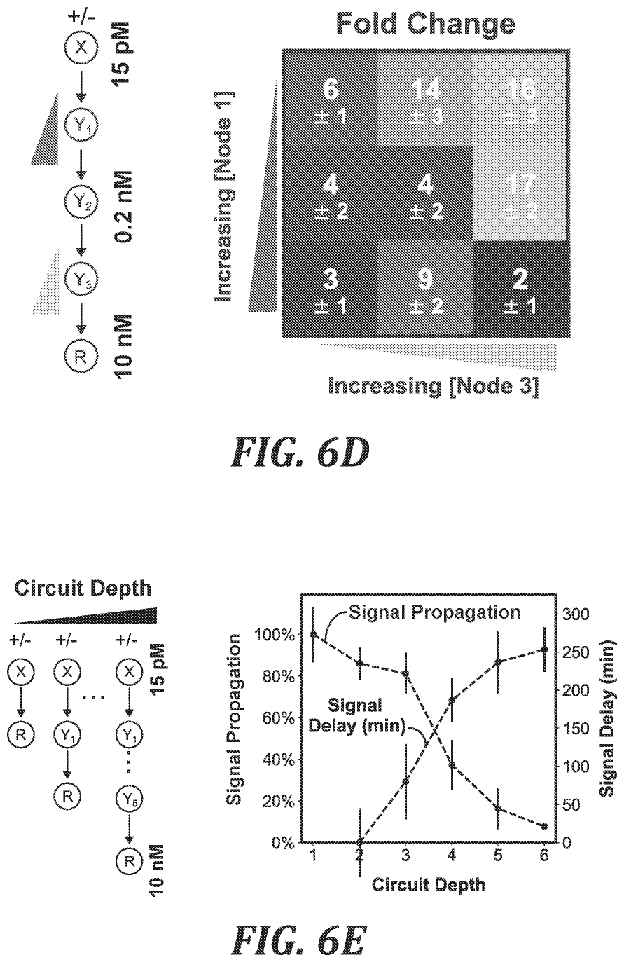

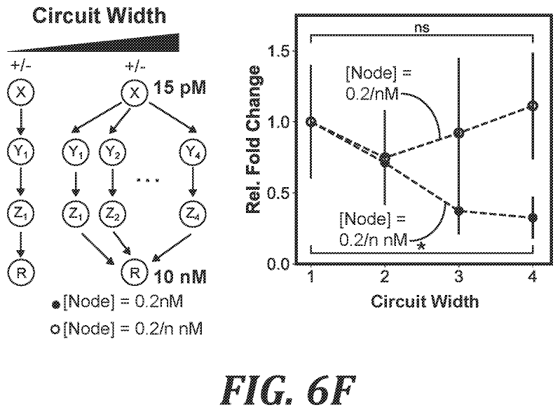

In embodiments of the synthetic GRN, the synthetic GRN comprises a feedback loop, a feedforward loop, a positive feedback, a negative feedback, a positive feedforward, and/or a negative feedforward. Components for these features can be selected and incorporated into the synthetic GRN based on their ability to activate or interfere with gene expression, for example, as described herein. In embodiments of the synthetic GRN, the propagation function implements a decreasing strategy, a constant strategy, an increasing strategy, or a data-informed strategy (see, for example, FIG. 6C and related description).

In embodiments of the synthetic GRN, the first promoter, the second promoter, or both, comprises an UP-element sequence that can be sub-Pareto optimal or at least approximately Pareto optimal for low basal transcription and high active transcription, and the first CRISPRa/i node, the second CRISPRa/i node, or both, comprises: a dCas9 polypeptide complexed, via a gRNA or a sgRNA, with a MCP polypeptide that is linked with a first heterodimer interaction domain, and a second heterodimer interaction domain that is linked with a SoxS domain. The gRNA or the sgRNA is configured to interact with the first promoter, the second promoter, or both to localize the first heterodimer interaction domain to the first promoter, the second promoter, or both, and the first heterodimer interaction domain is configured to conditionally interact with the second heterodimer interaction domain to localize the SoxS domain to the first promoter, the second promoter, or both. As a result of localization of the SoxS domain to a promoter, RNAP also localizes and initiates transcription from the promoter.

In embodiments, the synthetic GRN includes one or more CRISPRa/i nodes as described herein. For example, the one or more CRISPRa/i nodes of the synthetic GRN can implement as conditions irradiation of a matrix with a light that comprises a blue light element or contact of the matrix that comprises the CRISPRa/i node with a small molecule, and in instances where the blue light element is present in the condition, can include all or part of a EL222 light-responsive promoter system. Similarly, the synthetic GRN can include structural or functional features of the CRISPRa/i nodes of the disclosure, including but not limited to heterodimer interaction domains, GC content of the UP-element sequence, GC content of the minimal promoter sequence, GC content of the scRNA target sequence, and percent identities of elements of the one or more promoters with one or more sequence identifiers, as disclosed herein. In embodiments, the first promoter of the synthetic GRN has a higher dynamic range of transcription and the second promoter of the synthetic GRN has a higher absolute range of transcription. In embodiments, the synthetic GRN is at least one, two, three, four, five, or six layers deep, and is at least one, two, three, or four branches wide; in this manner, deeper and wider synthetic GRNs can be implemented for a wide range of applications.

### Kits and Methods

The disclosure also provides kits comprising one or more nucleic acids, one or more polypeptides, one or more cells, one or more cell-free systems, one or more biosensors, one or more biosynthesis systems, one or more biocomputational systems, or any combination thereof, optionally in combination with additional reagents or detection apparatuses or instructions for use, for example.

One or more nucleic acids comprising nucleotide sequences encoding one or more polypeptides, the gRNA or the sgRNA, one or more genes regulated by the CRISPRa/i node, the promoter, or any combination thereof, can be included alone or as part of a kit of the disclosure. The kit can include, as an example CFS, an E. coli CFS comprising polypeptides and the promoter. In embodiments, a kit can include a genetically modified host cell comprising one or more nucleic acids encoding the promoter, which can be a prokaryotic cell or a eukaryotic cell, for example.

In an aspect, the disclosure provides a method for control of transcription, the method comprising: providing a promoter comprising a minimal promoter sequence and an upstream element (UP-element) sequence that comprises a nucleotide base mutation for a modified promoter-RNA polymerase interaction to configure the promoter for a low basal transcription and a high active transcription; providing a non-dimerized EL222 transcription factor that comprises a first homodimer interaction domain and a second homodimer interaction domain, or contacting the CRISPRa complex to the promoter. The CRISPRa complex comprises a dCas9 polypeptide complexed, via a gRNA or a sgRNA, with a MCP polypeptide that is linked with a first heterodimer interaction domain, and the second heterodimer interaction domain is linked with a SoxS domain. The method further comprises meeting a condition that is associated with an interaction between the first homodimer interaction domain and the second homodimer interaction domain or the first heterodimer interaction domain and the second heterodimer interaction domain. When the condition is met, the interaction occurs, and an activation complex is formed for activation of transcription.

In embodiments, the providing the promoter comprises producing the promoter by: mutagenizing one or more nucleotide sequences of a reference promoter to produce one or more mutagenic libraries each comprising a plurality of candidate promoters; cloning the plurality of candidate promoters of the one or more mutagenic libraries onto a plurality of inducible reporter constructs; transforming the plurality of inducible reporter constructs into one or more host cells; measuring a basal level of transcription of the plurality of inducible reporter constructs by the one or more host cells; and measuring an activated level of transcription of the plurality of inducible reporter constructs by the one or more host cells. A difference between the activated level of transcription and the basal level of transcription can suggest that a high-performance activatable promoter has been produced. The complex of the method can be a component of a node as described herein.

In an aspect, the disclosure provides a method for producing a promoter comprising a minimal promoter sequence and an UP-element sequence that comprises a nucleotide base mutation for a modified promoter-RNA polymerase interaction to configure the promoter for a low basal transcription and a high active transcription. The method comprises: selecting a reference promoter; mutagenizing one or more nucleotide sequences of the reference promoter to produce one or more mutagenic libraries each comprising a plurality of candidate promoters; cloning the plurality of candidate promoters of the one or more mutagenic libraries onto a plurality of inducible reporter constructs; transforming the plurality of inducible reporter constructs into one or more host cells; measuring a basal level of transcription of the plurality of inducible reporter constructs by the one or more host cells; and measuring an activated level of transcription of the plurality of inducible reporter constructs by the one or more host cells. A difference between the activated level of transcription and the basal level of transcription can suggest that the promoter has either a higher dynamic range of transcription or a higher absolute range of transcription relative to corresponding metrics of the reference promoter.

In an aspect, the disclosure provides a method for producing a heterodimer CRISPRa system, the method comprising: providing a promoter comprising a minimal promoter sequence and an UP-element sequence that comprises a nucleotide base mutation for a modified promoter-RNA polymerase interaction to configure the promoter for a low basal transcription and a high active transcription; providing a first heterodimer domain linked to a MCP polypeptide; providing a second heterodimer domain linked to a SoxS domain; modifying a gRNA or a sgRNA; and testing the gRNA or the sgRNA for an ability of the gRNA or the sgRNA to activate transcription in combination with the promoter, the first heterodimer domain linked to the MCP polypeptide, and the second heterodimer domain linked to the SoxS domain. The ability of the gRNA or the sgRNA to activate transcription is associated with the structure of the gRNA or the sgRNA and a target location within the promoter. If the gRNA or the sgRNA sufficiently activates transcription in combination with the high-performance activatable promoter, the first heterodimer domain linked to the MCP polypeptide, and the second heterodimer domain linked to the SoxS domain, the heterodimer CRISPRa system is produced.

In embodiments, the method further comprises improving or optimizing the structure of the gRNA or the sgRNA with the target location within the high-performance activatable promoter to improve or optimize the ability of the gRNA or the sgRNA to activate transcription.

In embodiments, the providing the first heterodimer domain linked to the MCP polypeptide comprises linking the first heterodimer domain to the MCP polypeptide and the providing the second heterodimer domain linked to the SoxS domain comprises linking the second heterodimer domain to the SoxS domain.

### Integrations

In various aspects, a promoter, a CRISPRa/i node, a synthetic GRN, another component of the disclosure, or any combination thereof, can be integrated into a system or device for use. Non-limiting examples of such systems or devices include a synthetic biosensor, a synthetic bioreactor, and another type of synthetic biological system. The biosensor or bioreactor can include a hand-held form factor configured for use in the field or when taking environmental or clinical or non-clinical biomedical samples, for example, or can include an implantable form factor suitable for use in or with an implantable biomedical device. The form factor can be configured for long-term installation or use at a location, for example, a location in need of long-term monitoring of water or plant effluent for pollutants or contaminants.

As used herein, use of articles such as “a”, “an”, and “the”, for example, “an element”, refer to one element, at least one element, or one or more elements, unless something else is specifically stated. Similarly, plural forms include singular forms, unless something else is specifically stated.

As used herein in the description and claims, the term “comprising”, is inclusive or open-ended and does not exclude additional, unrecited elements or method steps. The term “consisting of,” as used in a claim, excludes any element, step, or ingredient not specified in the claim. The term “consisting essentially of,” as used in a claim, limits the scope of the claim to the specified materials or steps and those that do not materially affect the basic and novel characteristic(s) of the claim.

### EXAMPLES

### Example 1: Engineering Activatable Promoters for Scalable and Multi-Input CRISPRa/i Circuits

### Significance Statement

Gene regulatory networks expressed in cells or cell-free systems hold great promise for investigating the limits of biological information processing and developing platforms for molecular biosensing and chemical bioproduction. This example addresses the challenge of engineering gene regulatory networks that can dynamically activate many targets. The work described here enables new classes of deep, wide, and multi-input CRISPR-based genetic circuits. This example represents an important step towards engineered gene regulatory networks with complexities approaching those found in nature, with a wide range of applications in industry.

### Abstract

Synthetic, multi-layer CRISPR-based genetic circuits hold great promise for building gene regulatory networks for use in a wide range of industrial applications. This example develops an approach for creating high-performing activatable promoters that can be assembled into deep, wide, and multi-input CRISPR-activation and interference (CRISPRa/i) gene regulatory networks. By integrating sequence-based design and in vivo screening, activatable promoters are engineered that achieve up to 1000-fold dynamic range in an E. coli-based cell-free system. These new components enable CRISPRa gene regulatory networks that are six layers deep and four branches wide. The generalizability of the promoter engineering workflow is demonstrated by improving the dynamic range of the light-dependent EL222 optogenetic system from 6-fold to 34-fold. Additionally, high dynamic range promoters enable CRISPRa systems mediated by small molecules and protein-protein interactions. These tools are applied to build input-responsive CRISPRa/i gene regulatory networks, including feedback loops, logic gates, multi-layer cascades, and dynamic pulse modulators. This example provides a generalizable approach for the design of high dynamic range activatable promoters and enables new classes of gene regulatory functions in cells and cell-free systems.

### INTRODUCTION

Gene regulatory networks (GRNs) can sense diverse environmental cues and respond to them through the coordinated expression of multiple genes. Cell-free systems (CFS) have emerged as an attractive chassis for building synthetic biological systems as they allow for rapid prototyping of genetic parts and circuits. To build increasingly complex CFS capable of sensing and responding to diverse inputs, new approaches for increasing the capabilities of synthetic GRNs are needed. The advances in GRN engineering disclosed herein accelerate the use of CFS for building multiplexed biosensors, deploying on-demand bioproduction platforms, and the construction of synthetic cells.

CRISPR-Cas transcriptional regulation has proven a promising framework for building sophisticated genetic circuits across a variety of biological systems. Transcriptional units containing target sequences for CRISPR activation (CRISPRa) and/or CRISPR interference (CRISPRi), termed CRISPRa/i nodes, can be assembled into circuits with network topologies specified by guide RNAs (gRNAs). This example's experimental and theoretical analyses indicate that the CRISPRa/i system is well suited for the design of deep and wide control circuits containing internal nodes connected in series or parallel through orthogonal gRNAs. For example, large CRISPRi GRNs with up to 7 gRNAs have been constructed in yeast by implementing low leak promoters and high dynamic range repressors. In E. coli-based CFS, CRISPRi genetic control is established, and CRISPRa has recently been incorporated, greatly expanding the circuit design space. However, CRISPRa circuitry is greatly limited by a lack of promoter-gRNA pairs that can be interconnected with minimal signal degradation. Hence, the disclosed generalizable approaches for engineering activatable promoters with low basal and high activated expression levels serve to significantly improve CRISPRa and enable the assembly of complex, input-responsive GRNs for a wide range of applications in industry, including but not limited to biosensors and bioproduction.

Promoter engineering efforts have previously focused on designing constitutive and inducible promoters with predictable expression characteristics. Tuning the strength of constitutive promoters involved designing promoter sequences that achieve a desired level of RNA polymerase (RNAP) recruitment to the promoter. Inducible promoters contain recognition sites for transcriptional activators or repressors that modulate transcriptional levels upon binding. Development of high dynamic range inducible promoters has relied on engineering de-repression-based systems, largely due to the difficulty of rationally designing activatable promoters. For effective activation, RNAP recruitment to the promoter should be weak in the absence of an activator, however, transcription initiation should be strong upon activator-mediated RNAP recruitment. Hence, tuning RNAP interactions through promoter sequence design could lead to higher dynamic ranges with CRISPRa and other transcriptional activation systems.

Approaches integrating sequence-based design and in vivo screening are described herein and used to generate an expandable set of high-performing promoters that exhibit both low basal and high activated expression levels. Through a sequential selection approach, activatable promoters with up to 1000-fold dynamic range are designed, constituting a 33-fold improvement from previous synthetic promoters. These promoters enable complex network topologies with performance levels not previously accessible in CFS, including a six-layer deep cascade and a four-branch parallel circuit. By engineering activatable promoters, different inputs can be incorporated into CRISPRa/i CFS circuits. It is demonstrated that a blue light-responsive transcriptional activator and three different protein-protein interaction (PPI)-dependent CRISPRa systems can function as circuit inputs. Input-responsive CRISPRa/i circuits operating as multi-layer activation cascades, positive feedback loops, AND-like logic gates, and dynamic two-input pulse modulators are engineered. Overall, this example describes a new workflow for engineering activatable promoters and provides a toolbox of versatile components with immediate utility for implementing CRISPRa/i circuits. Together, these developments dramatically expand the ability to assemble large, multi-input GRNs in CFS.

### Results

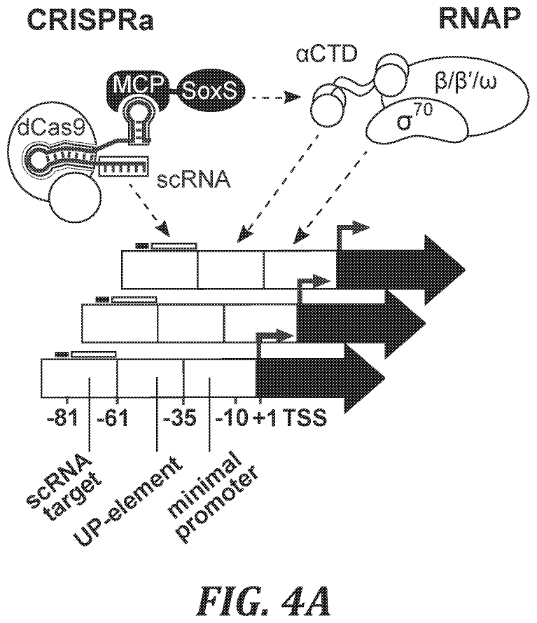

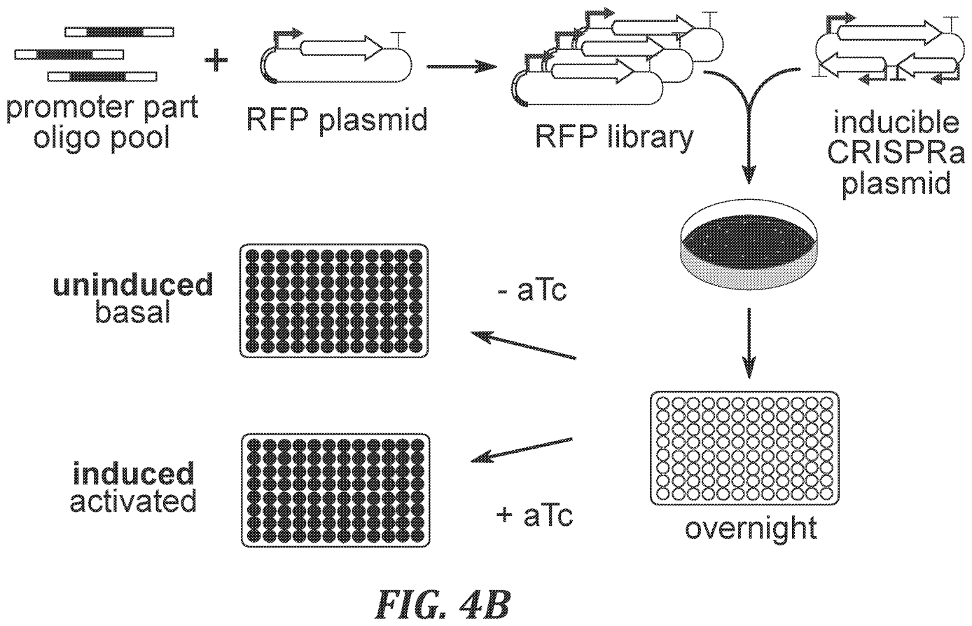

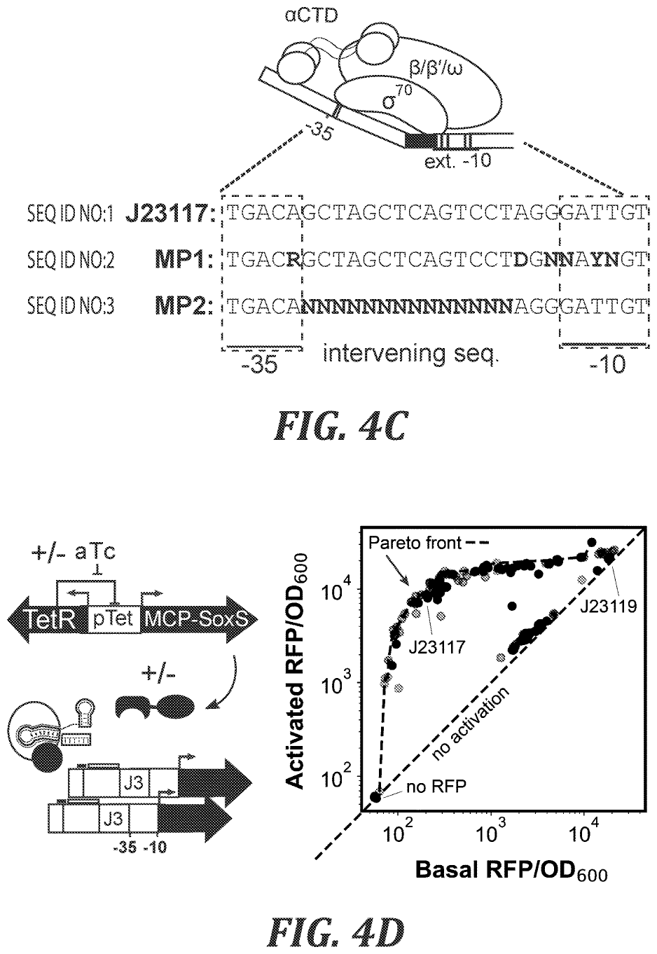

It was first sought to characterize the impact of sequences affecting RNAP recruitment on both basal and activated expression levels of synthetic activatable promoters. RNAP recruitment is dependent on the affinity of the RNAP sigma subunit (G) for the −10 and −35 hexamers of the minimal promoter (FIG. 4A). Additionally, recruitment is influenced by the GC-content of the intervening sequence between the −10 and −35 sites (FIG. 4A). Promoter recognition is enhanced by the AT-rich UP-elements upstream of the minimal promoter, which anchor the α-subunits of RNAP. Collectively, the sequence compositions of these regions influence RNAP recruitment, binding, and initiation at the promoter. Libraries of these discrete promoter regions were systematically designed, and a scalable workflow was used for screening and isolating library variants in E. coli. The libraries were co-transformed with an aTc-inducible CRISPRa plasmid to enable parallel screening of basal and activated expression levels (FIG. 4B, Example 1 Materials and Methods). In the absence of aTc, RNAP recruitment is determined by the promoter basal strength. Upon aTc induction, the MCP-SoxS activator is expressed and localized to a CRISPRa complex at the promoter via a modified gRNA, or scaffold RNA (scRNA), containing the MS2 hairpin. MCP-SoxS then recruits RNAP to the promoter through α-CTD interactions, activating transcription (FIG. 4A). This approach allows to characterize the impact of individual promoter regions on basal and activated expression simultaneously, and combine variants with low basal and high activated expression to construct high-performing activatable promoters.

Functional Interrogation of Promoter Regions with CRISPRa

### Impact of Minimal Promoter Region on Activatability

The minimal promoter region is important in determining basal and activated expression levels. Two minimal promoter libraries were designed, mutagenizing the −10/−35 hexamers and the intervening sequence of the previously identified best-performing minimal promoter (BBa_J23117) within the J3 synthetic CRISPRa promoter (FIG. 4C, Example 1 Materials and Methods). These libraries were co-transformed with the aTc-inducible CRISPRa plasmid expressing the J306 scRNA targeting the cognate J3 promoter. Both libraries yielded high promoter diversity, as measured by RFP expression levels, with basal and activated expression levels ranging from that of a no-reporter control to a strong constitutive promoter (BBa_J23119) (FIG. 4D). Without wishing to be bound by any particular theory, the subset of promoters not activated could arise from the generation of sequences resembling tightly regulated native promoters.

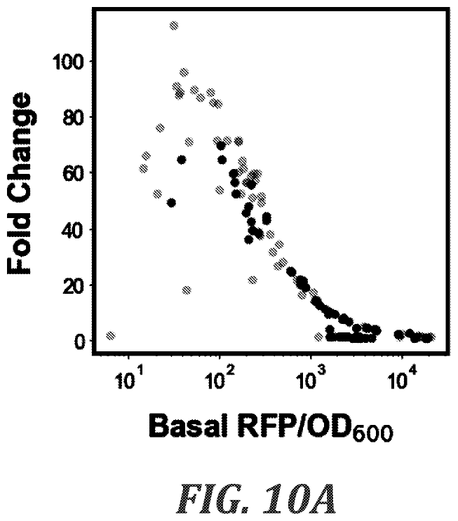

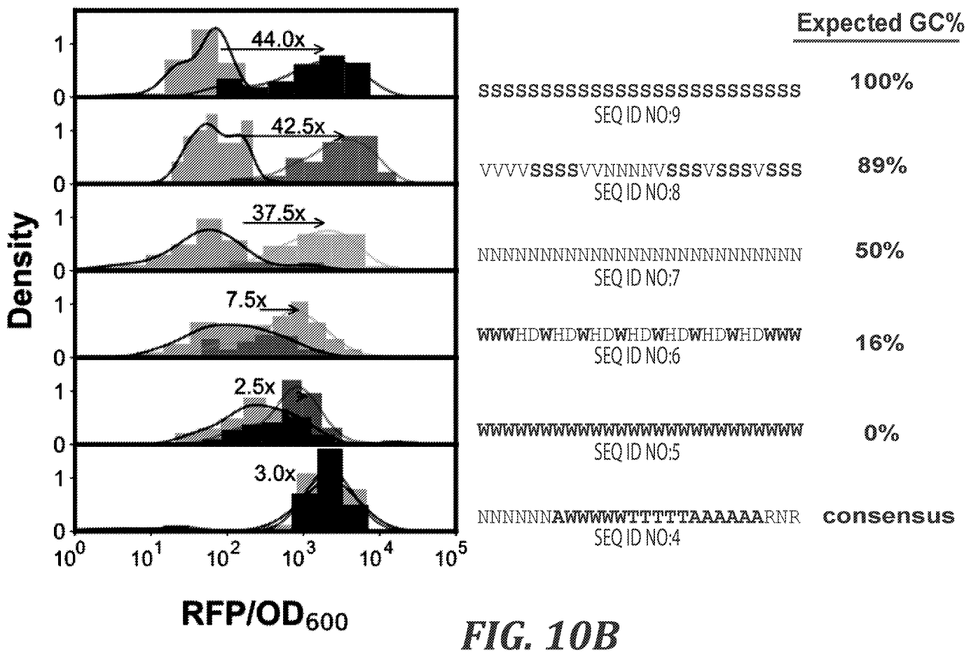

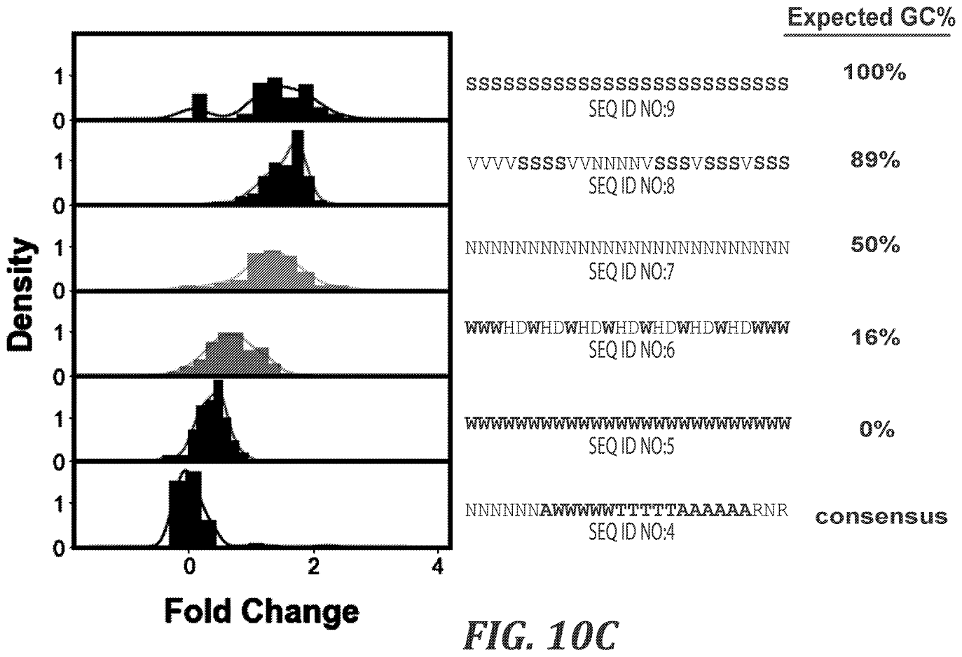

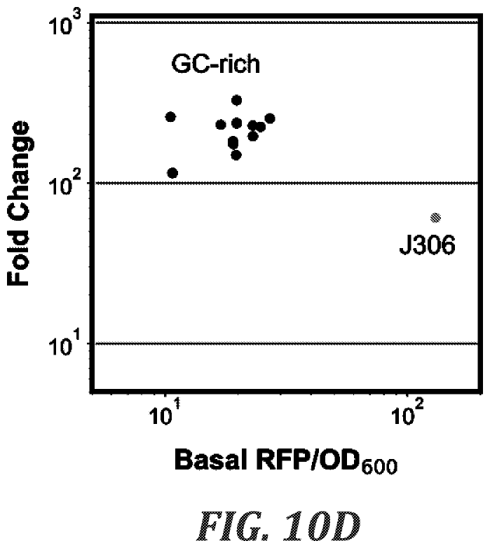

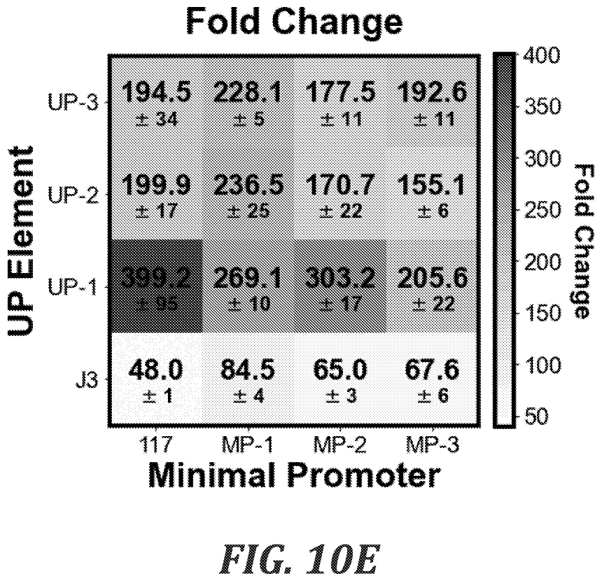

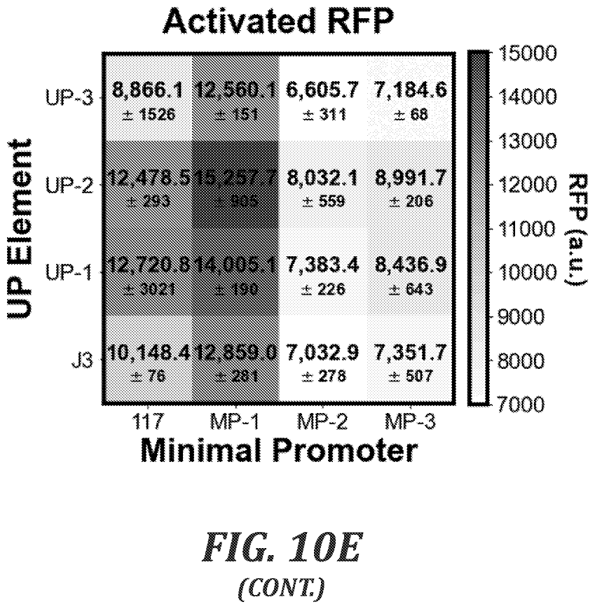

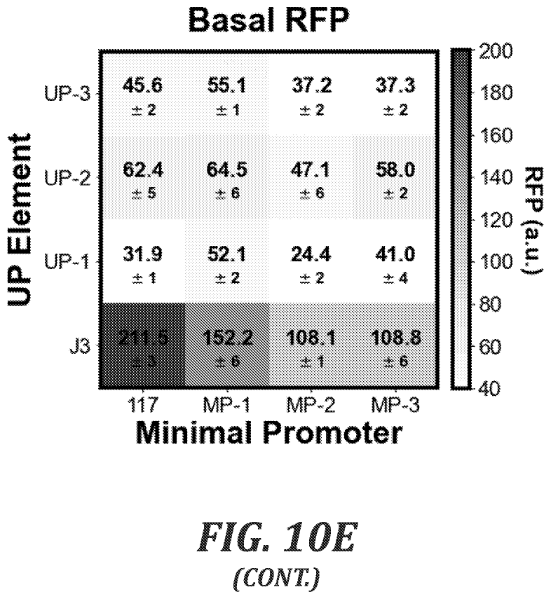

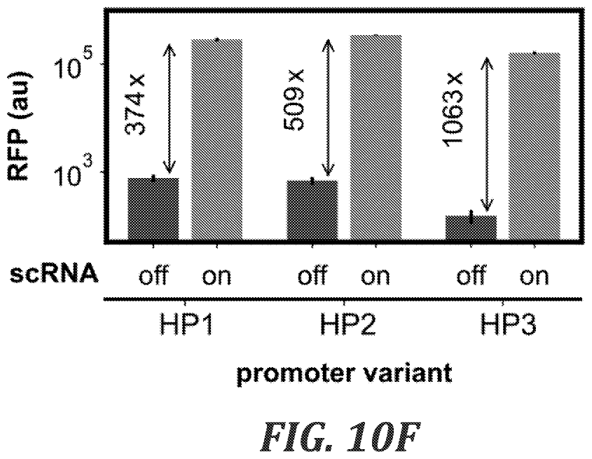

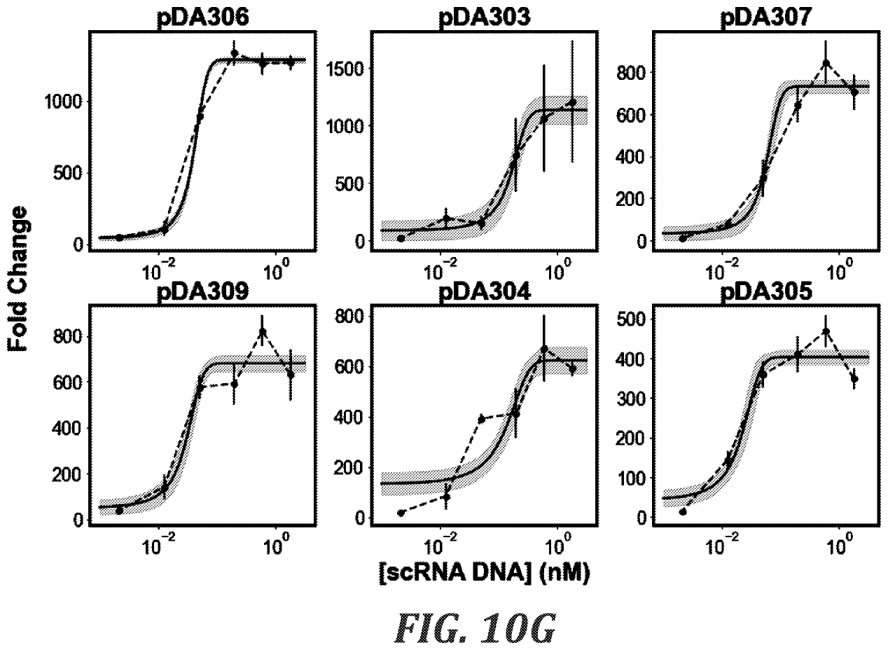

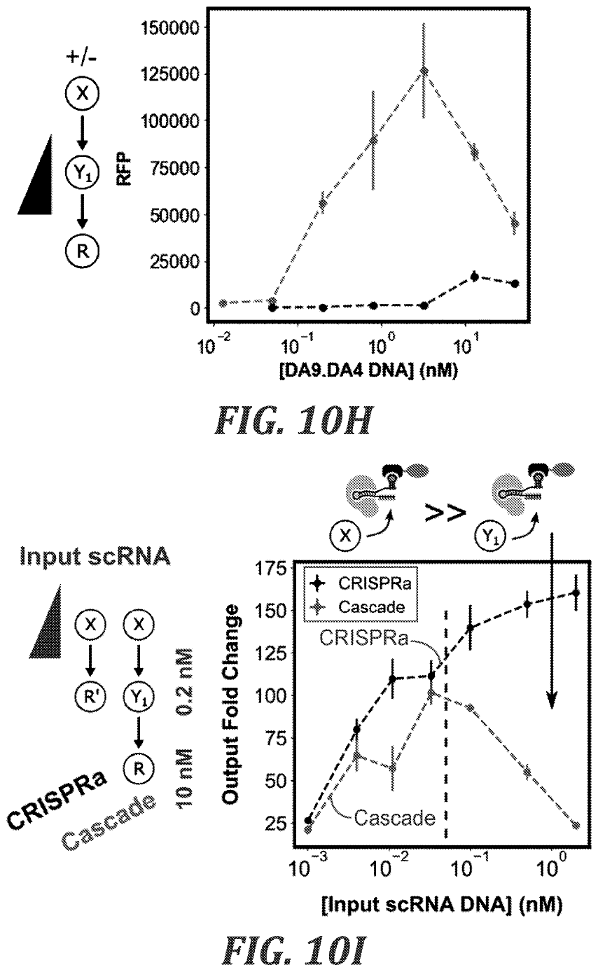

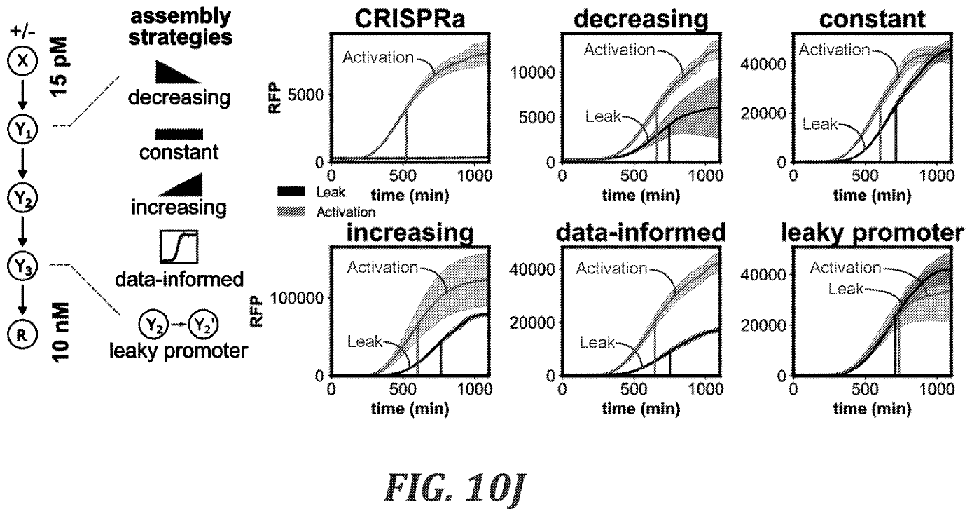

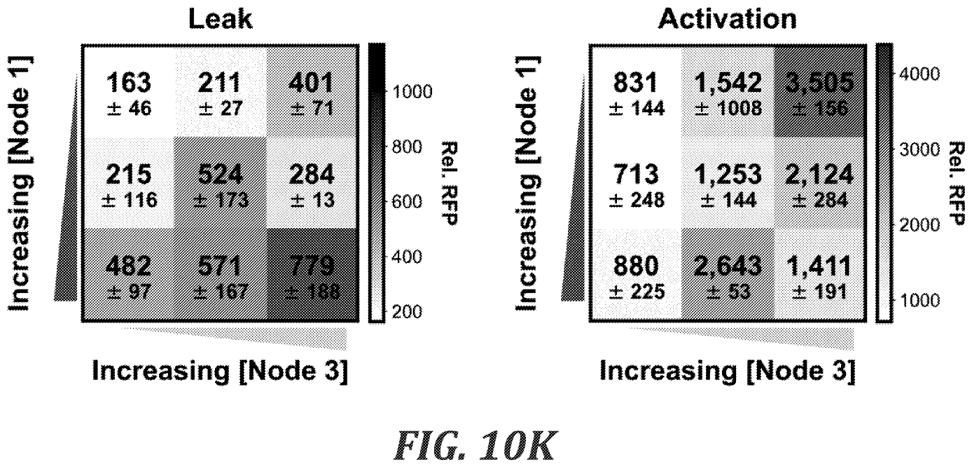

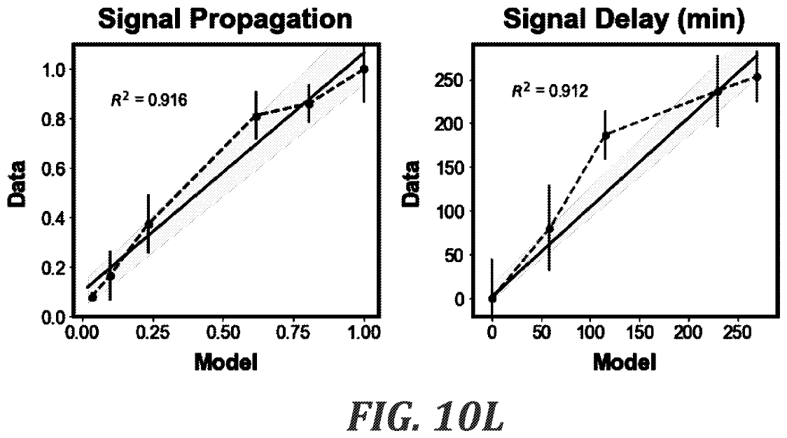

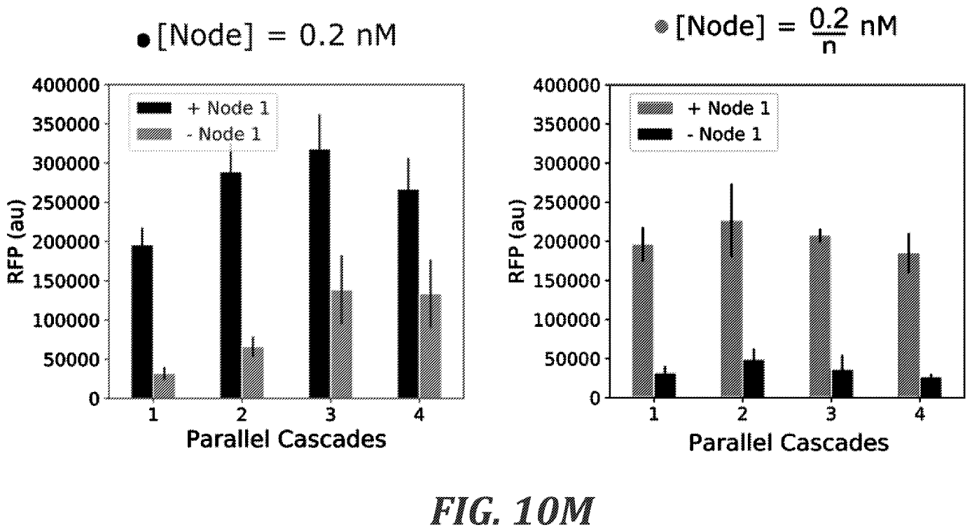

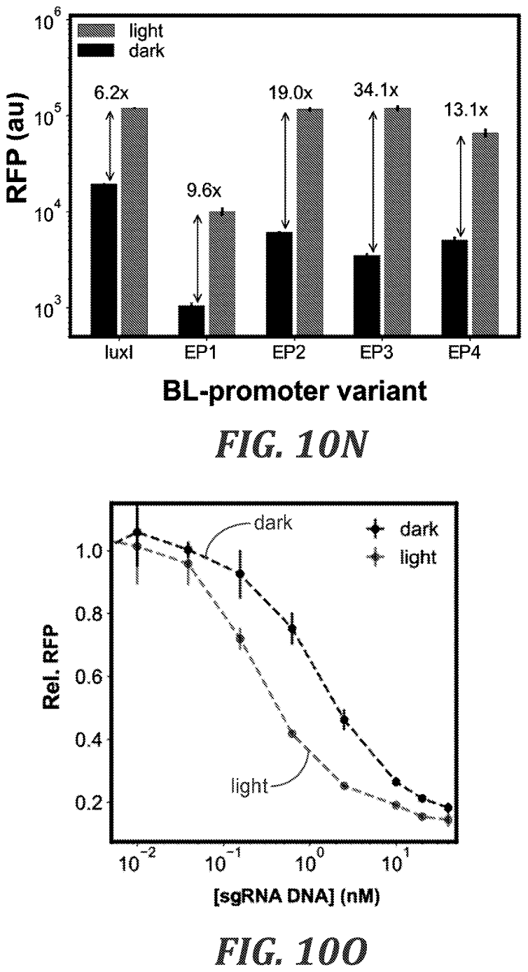

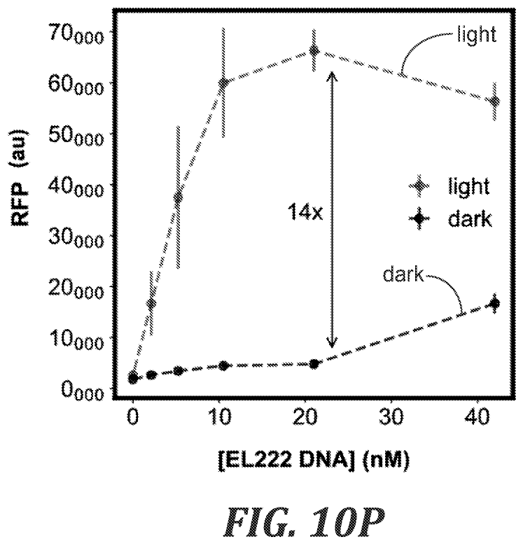

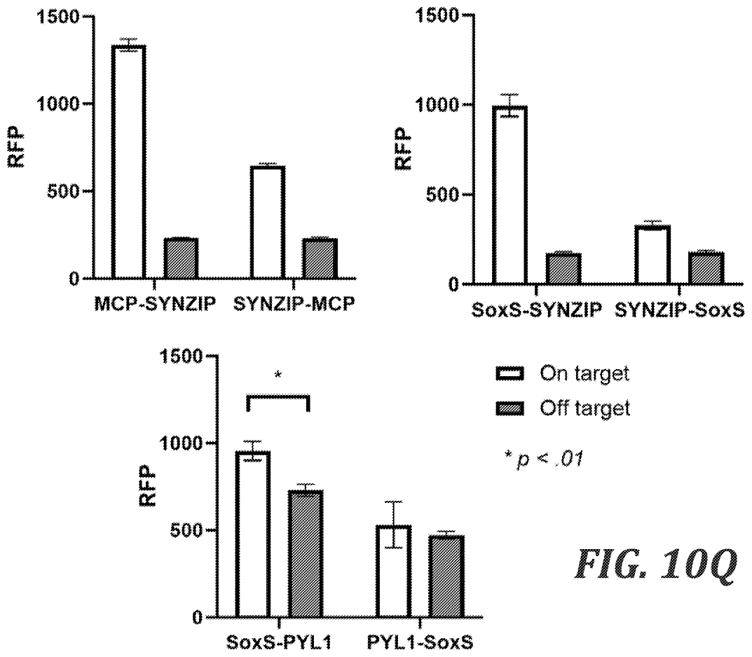

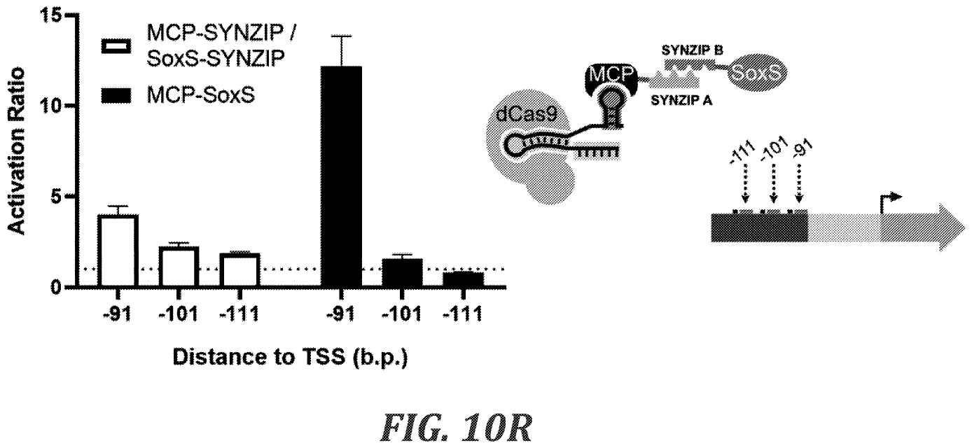

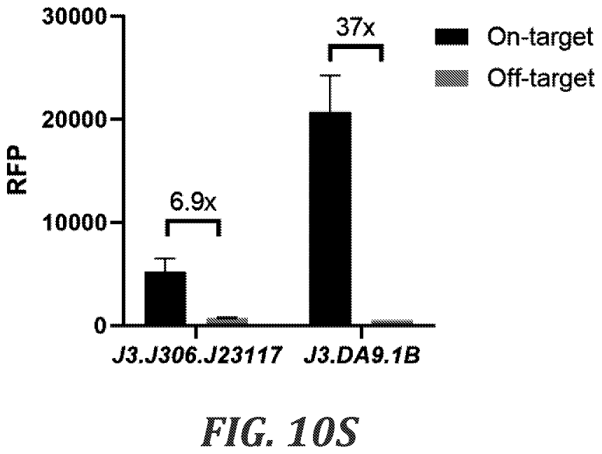

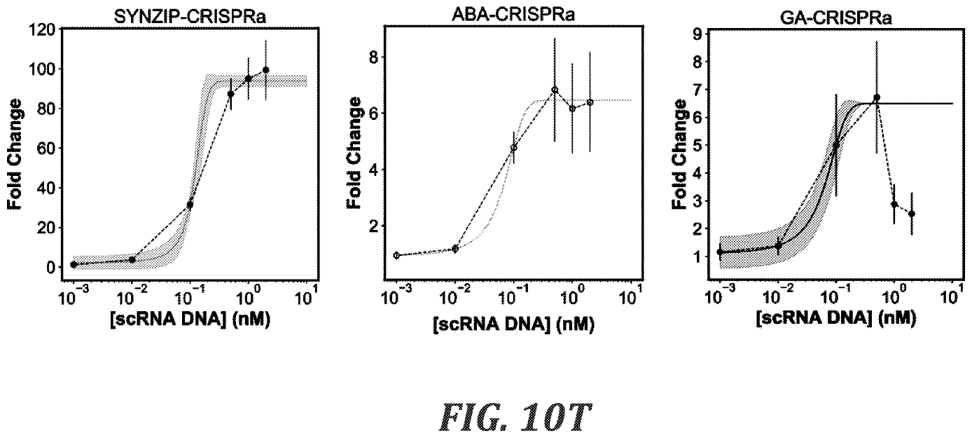

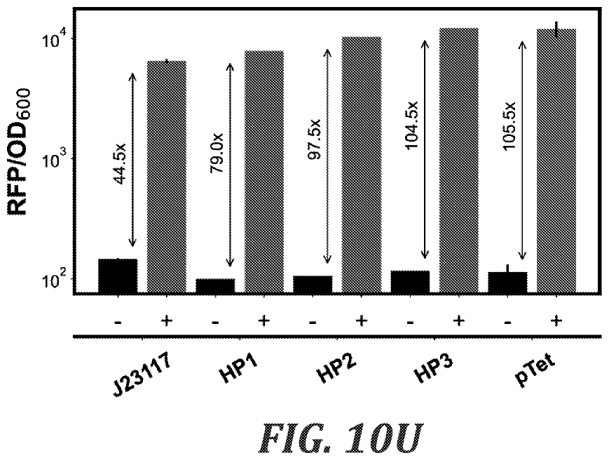

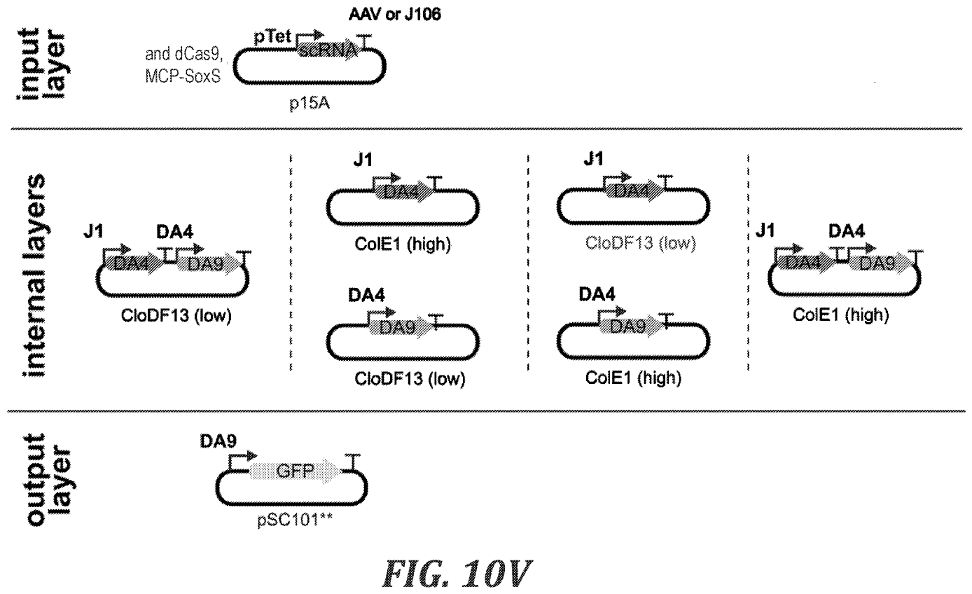

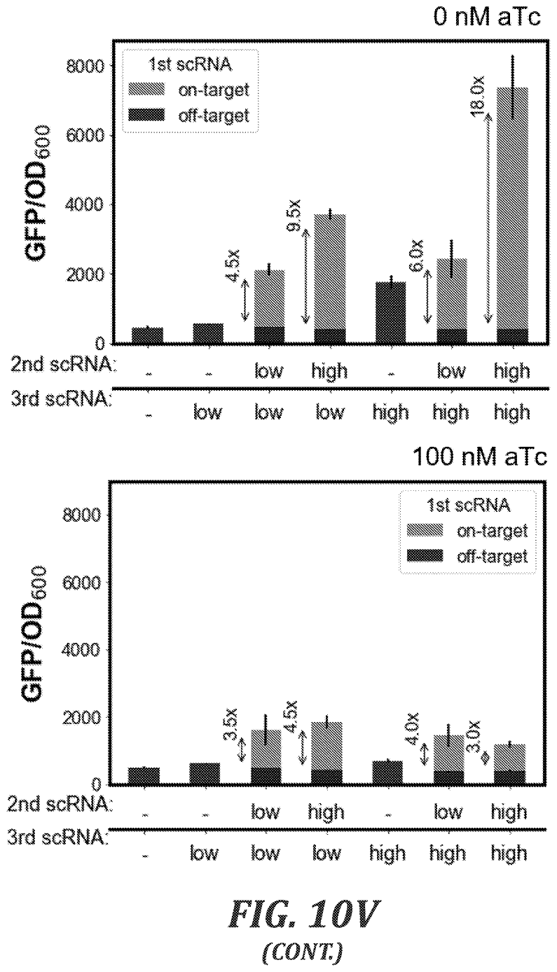

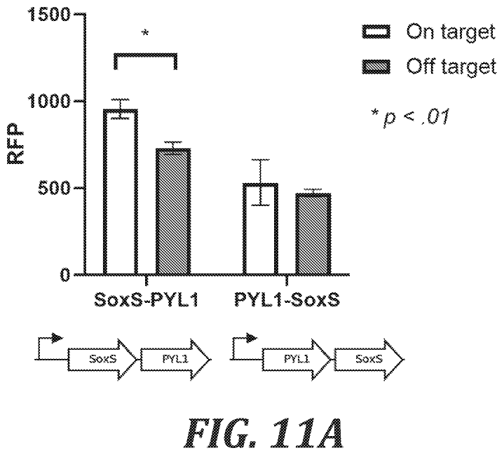

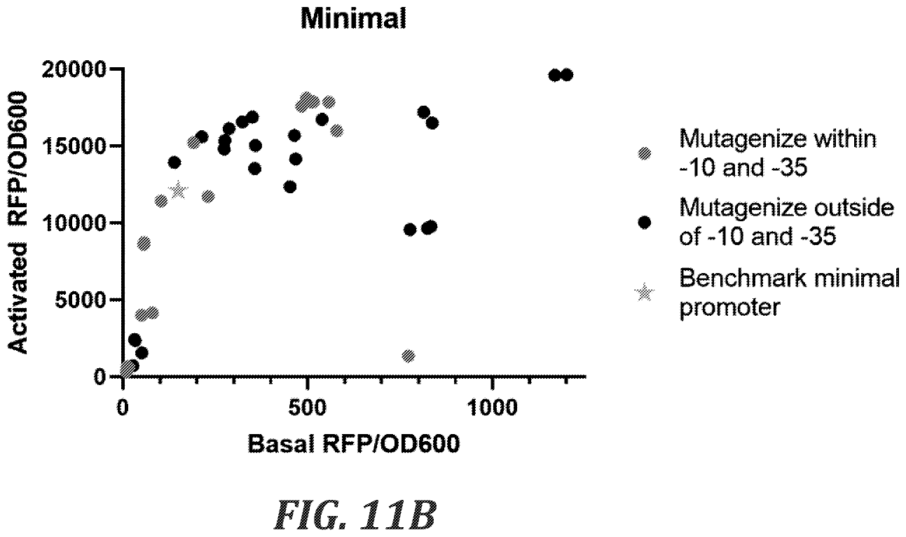

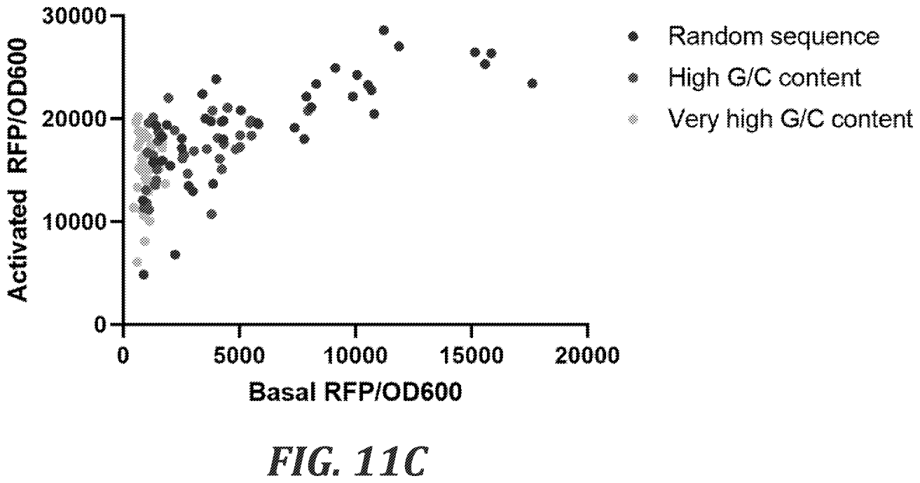

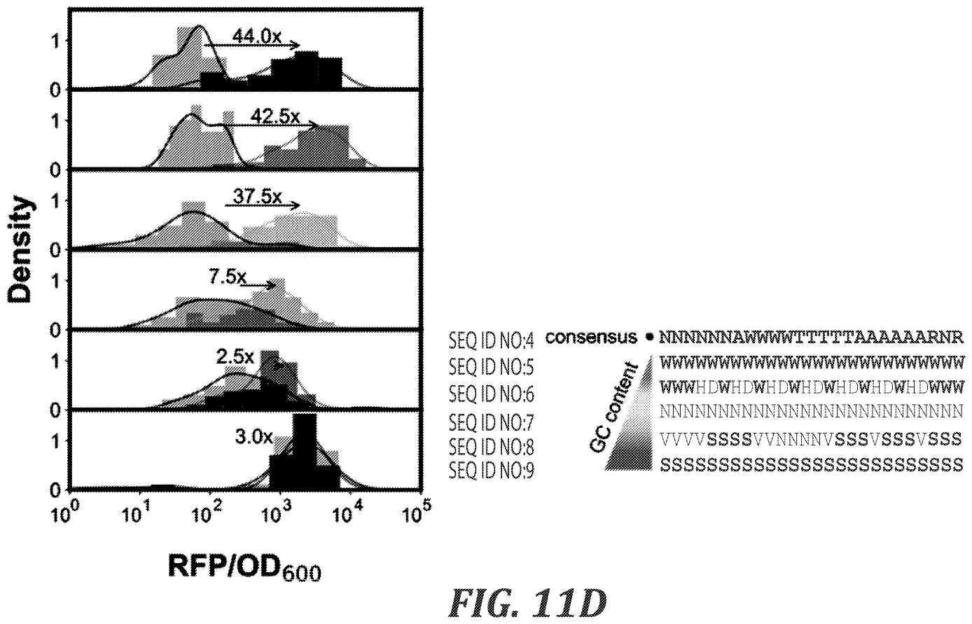

The set of minimal promoter variants that maintain both low basal and high activated expression levels can be conceptualized as a Pareto optimal front. In multi-objective optimization, a Pareto front defines the best-performing solutions for which no further improvements in either objective can be achieved without compromising the other (Example 1 Materials and Methods). Three variants from this Pareto front exhibited both lower basal and higher activated expression levels compared to the original BBa_J23117, indicating the original minimal promoter was not a part of the Pareto front. This finding suggests that promoter mutagenesis can yield improved activatable promoters beyond previous screening methods based on promoter basal strengths alone. By mutagenizing the minimal promoter of CRISPRa promoters, sequences with >100-fold dynamic range in CRISPRa-mediated gene expression were generated (FIG. 10A).

### Impact of UP-Element Region on Activatability

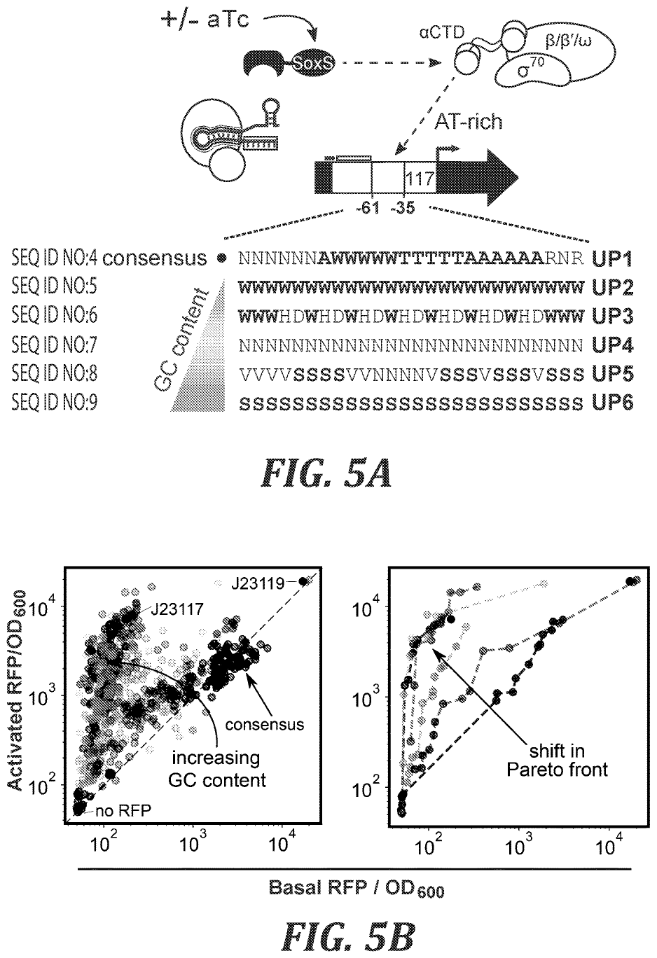

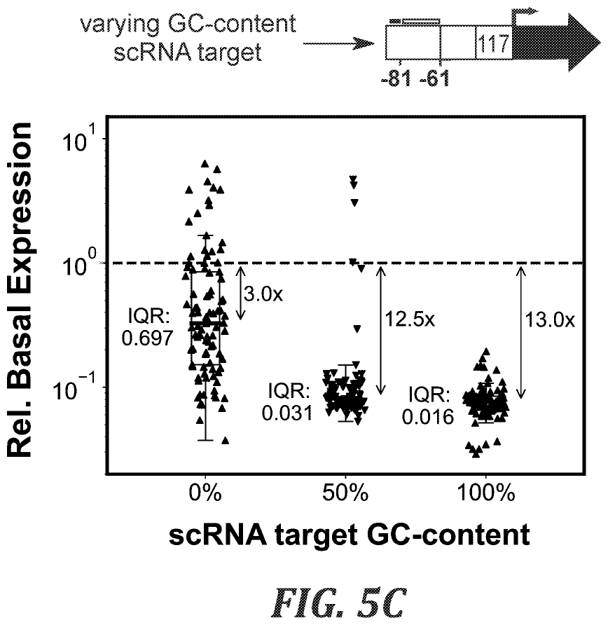

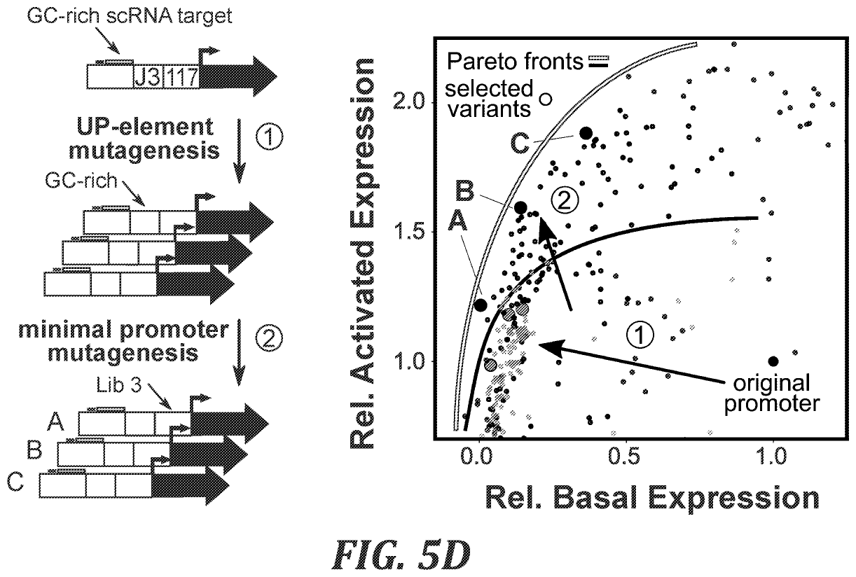

RNAP promoter recognition is enhanced by the AT-rich UP-elements upstream of the minimal promoter, which anchor the α-subunits of RNAP. For effective CRISPRa, RNAP should only be recruited to the promoter in the presence of an on-target scRNA. Hence, for transcriptional activation with SoxS, improvements in dynamic range could be achieved by minimizing RNAP-UP-element interactions and lowering basal expression levels. Five UP-element libraries mutagenizing the AT-rich E. coli consensus sequence with increasing GC-content were generated (FIG. 5A, Example 1 Materials and Methods). The consensus UP-element and the AT-rich library had the highest basal expression levels (FIG. 5B, left). On average, these libraries showed 3-fold activation, as compared to 37- to 44-fold activation for the more GC-rich libraries (FIGS. 10B and 10C). Qualitatively, a monotonic decrease in basal expression levels was observed and there was no impact on maximum activated levels with increased GC-content (FIG. 10B). The optimal variants from each library were identified and a shift in the Pareto front towards lower basal and higher activated expression levels with increasing UP-element GC-content was found (FIG. 5B, right). Specifically, the median basal and activated expression levels of the GC-rich optimal variants were 59.7-fold lower and 1.7-fold higher than that of the AT-rich optimal variants. The original J3 synthetic promoter, with an UP-element GC content of 50%, sat in between the Pareto fronts that included the high and low GC-content variants. By mutagenizing the UP-element, promoter variants with >350-fold dynamic range in CRISPRa-mediated gene expression were generated.

Impact of the scRNA Target Site Region on Activatability

Transcriptional activators bind upstream of the minimal promoter region to recruit RNAP to the transcription start site (TSS). For CRISPRa, the optimal scRNA target site location for SoxS-mediated activation is-81 bp upstream of the TSS. Changing the scRNA target sequence enables rapid generation of orthogonal CRISPRa promoters. Due to the proximity to the UP-element region, it was reasoned that the sequence composition of the scRNA target site can have an impact on basal expression levels. Three scRNA target site libraries composed of varying GC-content were designed (Example 1 Materials and Methods) and the basal expression of each library was measured. GC-rich libraries had 4.3-fold lower median basal expression compared to AT-rich libraries (FIG. 5C). Additionally, the spread of the basal expression decreased monotonically with increasing GC-content of the scRNA target site sequence (FIG. 5C). Without wishing to be bound by any particular theory, these results could indicate GC-rich scRNA target site sequences lead to low basal expression CRISPRa promoters, possibly due to reduced interaction with RNAP. To validate the CRISPRa activity at these low basal expression scRNA target sites, 10 GC-rich variants were selected and the corresponding scRNAs constructed. All variants produced a higher fold-activation than the original J306 scRNA (FIG. 10D), with 3.5-fold average increase in fold-activation.

### Combining Promoter Regions to Engineer High-Performing CRISPRa Promoters

### Engineering Activatable Promoters by Combining Optimized Promoter Regions

Analysis proceeded to test if the highest performing variants from the UP-element and minimal promoter screens could be combined to yield activatable promoters with improved performance. Three high-performing variants from both the UP-element and minimal promoter screens were selected, as well as the starting J3 UP-element and BBa_J23117 minimal promoter, and a combinatorial set of 16 promoters was constructed. Notably, promoter regions that gave the largest improvements in the original context did not necessarily give the largest improvements when tested in different contexts (FIG. 10E). For instance, an UP-element that gave 300-fold activation with one minimal promoter only gave 200-fold activation with a different high-performing minimal promoter (p=0.003), largely due to an increase in basal expression (FIG. 10E). ANOVA results (p=10⁻⁵) support the view that UP-element and minimal promoter contributions affecting activation cannot be isolated from one another (Table 7).

### Engineering Activatable Promoters Through Sequential Promoter Region Screening

The results from the previous section highlight the effects that promoter region contexts play in the design of high-performing activatable promoters. Therefore, it was tested whether promoters with improved basal and activated expression levels could be achieved by selecting minimal promoters in the context of high-performing UP-elements. The UP-element region was first screened as these libraries had a larger impact than the minimal promoter libraries on the location of the Pareto front (FIGS. 4D and 5B). The UP-element of a promoter containing the minimal promoter BBa_J23117 and a GC-rich scRNA target was mutagenized. Three Pareto-optimal UP-element variants which had on average 10% of the original basal and 12% higher activated expression than the J3 promoter were selected (FIG. 5D). Minimal promoter libraries were then screened in the context of these three selected UP-element variants. Three new promoter variants were selected from the Pareto front, which had on average 17% of the basal and 56% higher activated expression than the J3 promoter (FIG. 5D). With this sequential screening approach, the context effects observed in the previous section were overcome and the Pareto front was successfully shifted towards lower basal and higher activated expression for both the UP-element and minimal promoter screens.

Engineering Deep and Wide Circuits with High-Performing CRISPRa Promoters

In nature, interconnected, multi-layer transcriptional networks coordinate the timing and levels of expression of multiple genes to produce complex responses to environmental stimuli. To develop CRISPRa/i GRNs with complexities approaching those found in nature, components enabling construction of circuits with arbitrary interconnections and minimal signal degradation are needed. CRISPRa promoters with high output dynamic ranges can minimize signal degradation by enabling effective level-matching of the input/output dynamic ranges between sequential CRISPRa/i nodes. Dynamic range improvements achieved at the promoter level can translate into signal propagation improvements at the circuit level and enable construction of increasingly deep and wide CRISPRa circuits.

### Engineering Functional CRISPRa/i Nodes

The three promoter variants selected above (FIG. 5D, HP1-3; A-C) were characterized in CFS and up to ~1000-fold dynamic range was observed (FIG. 10F) and a high degree of correspondence with in vivo expression levels (R²=0.92). These promoters were used to generate a set of orthogonal CRISPRa/i nodes to be assembled into multi-layer circuits. The highest dynamic range activatable promoter (HP3, Table 1) was combined with previously-screened scRNA target sites to generate orthogonal CRISPRa/i nodes (FIG. 6A). Dose-response curves for each orthogonal node made by titrating the plasmid expressing the cognate scRNA showed that these new nodes gave an average activation of 890-fold (FIGS. 6B, 10G; Table 1). Given the context dependence of activation, these nodes can be further improved through sequential mutagenesis of promoter elements in the context of the corresponding scRNA target sequence.

### Deep CRISPRa Circuits

It was investigated whether deep multi-layer cascades could be implemented using the improved CRISPRa/i nodes. First a two-layer CRISPRa cascade was built by tuning the expression levels of the input and internal CRISPRa/i nodes and up to 127-fold activation was achieved (FIGS. 10H, 10I). An excess of either the input or internal node led to decreased performance of the cascade, potentially due to scRNA competition for binding to dCas9. Next, four-layer activation cascades were assembled. To compare circuit performance and dynamics in response to scRNA inputs, RFP expression and time to maximum expression rate (Tₘₐₓ) were measured (FIGS. 6C, insert; FIG. 10J) (Example 1 Materials and Methods). If the input signal propagates faster than the leak from the rest of the nodes, CRISPRa-dependent expression through the network accelerates, reducing tₘₐₓ. Therefore, a larger tₘₐₓ between the +/−input conditions (ΔTₘₐₓ) corresponds with improved circuit function.

For a cascade with equal node concentrations at each layer, no input-dependent change in maximum RFP expression was found, and a Δ t_{maX} of 110 min was observed (FIG. 6C, middle). Node concentrations were tuned by either decreasing (denoted “D”) or increasing (denoted “I”) the concentration of each subsequent node as depth increased (FIG. 3C, left). These Assemblies D and I had the lowest and highest expression levels, respectively (FIG. 6C, middle). These assemblies also had tₘₐₓ significantly accelerated compared to their no input conditions (Δ tₘₐₓ of 85 min and 165 min, and p=0.01 and p=3×10⁻⁴, respectively) (FIG. 6C, right). When scRNA-promoter dose-response curves were used to inform circuit assembly, 3-fold higher activated expression was obtained compared with assembly D and 4.5-fold lower basal expression was obtained compared with assembly I, with a similar overall fold-activation and Δ tₘₐₓ (FIG. 6C).

The impact of high-performing promoters on circuit function was interrogated by exchanging the high-performing promoter of the second internal node for a previously characterized promoter with leakier basal expression. While the leakier promoter was sufficient for constructing functional two-layer cascades, the four-layer cascade was no longer input-responsive, as indicated by the fact that expression level and tₘₐₓ were the same with or without input scRNA. In contrast, all of the assemblies containing high-performing promoters were input-responsive, underscoring the importance of high-performing promoters for building deep transcriptional circuits.

To improve the fold-activation of the four-layer cascade, the concentrations of individual nodes in the cascade were tuned. To reduce the experimental search space, the second node was held constant and the concentrations of the first and third internal nodes were tuned. With this approach, the tuned four-layer cascade achieved 16-fold activation compared to 2-fold activation of the initial cascades (FIGS. 6C and 6D). Qualitatively, higher concentrations of the third node resulted in higher activated states, while lower concentrations of the first node minimized basal expression (FIG. 10K). Notably, these circuits maintained roughly 80% of their maximal activity even when node concentrations varied by nearly 50% (FIGS. 6D and 10K), showing that they are robust to changes in the amounts of gRNAs and targets. Overall, these results indicate that timing and expression level of multi-layer, input-responsive circuits can be controlled through node concentration tuning.

It was investigated how input signals are communicated through increasingly deep circuits by measuring signal propagation and signal delay at each layer (FIG. 6E, left). The percentage of signal propagated was quantified by calculating the fold-activation of the full cascade output divided by the activation from the input layer (Example 1 Materials and Methods). Signal delay was the difference between t_{maxFC} of the cascade input and output layer, where t_{maxFC} is the time needed to reach the maximum fold-activation (Example 1 Materials and Methods). Signal propagation was sustained above 80% until the 4th layer was added, after which it decreased rapidly (FIG. 6E, right). Nevertheless, measurable output differences in circuits of up to six layers was observed. The two-layer cascade gave no significant difference in signal delay compared to a single-layer CRISPRa reaction (p=0.9). This may suggest there is a slow step in output production, such as fluorophore maturation, that masks the effect of the second layer. Beyond two layers, the signal delay increased with subsequent additions of the third, fourth, and sixth layers (FIG. 6E, right), averaging ~50 min/layer, but ranging from ~20-100 min.

It was investigated whether the signal propagation and signal delay at each layer could be explained by the performance of individual nodes. The maximum fold-change of individual nodes from the dose-response curves (FIG. 10G) was used to predict the delay and signal propagation at the subsequent level (Example 1 Materials and Methods). The model-derived predictions showed high correspondence with the signal propagation and delay at each of the six layers (R²=0.92 and R²=0.91, respectively) (FIG. 10L). These results are consistent with the proposal that high-performing nodes can quickly activate subsequent layers, propagate signals efficiently, and be assembled into deep circuits.

### Wide CRISPRa Circuits

To identify conditions under which orthogonal nodes can compose wide CRISPRa circuits, one, two, three, and four parallel three-layer cascades operating in the same CFS reaction were constructed. A single input was used to activate the downstream nodes, and circuit performance was measured by connecting all of the parallel cascade scRNA outputs to the same RFP node (FIG. 6F, left). When the internal node concentrations were maintained constant across parallel cascades, up to 66% decrease in output fold-activation was observed as the width of the circuit increased from one to four cascades (FIG. 6F, right). This decrease came largely from higher output levels in the absence of scRNA input (FIG. 10M), possibly due to higher basal expression of internal scRNAs. The same circuits were then constructed and the node concentrations tuned proportionally to the number of parallel cascades, effectively maintaining the total node concentration constant. When constructed in this manner, no statistically significant difference in the fold-activation across cascades of different widths was found (p=0.61) (FIG. 6F, right). Hence, by tuning the concentration of orthogonal CRISPRa/i nodes, it is shown that an arbitrary number of parallel circuits with as many as nine nodes can be regulated.

### Developing Activatable Promoters for Blue Light Responsive CRISPRa/i Circuits

### High-Performing Blue Light Responsive Promoters

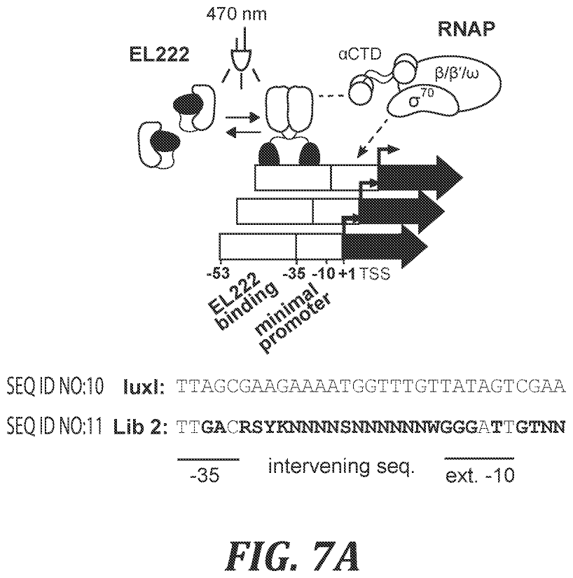

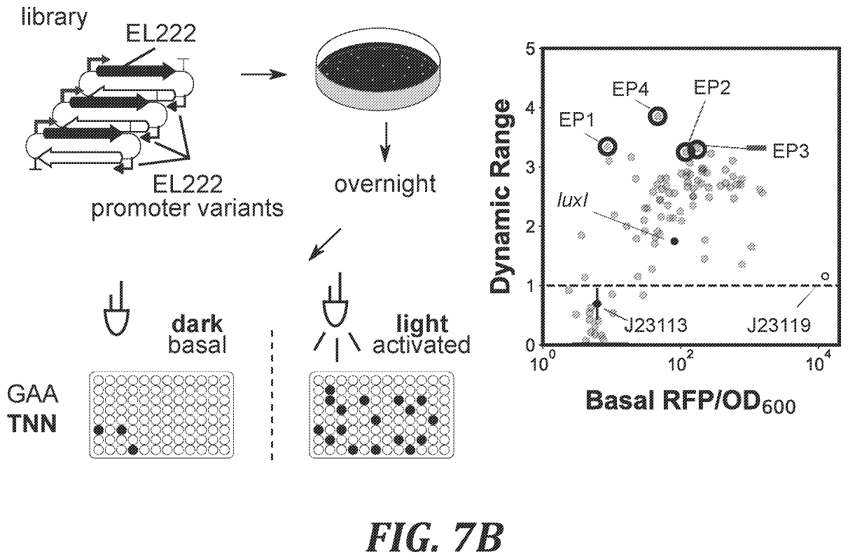

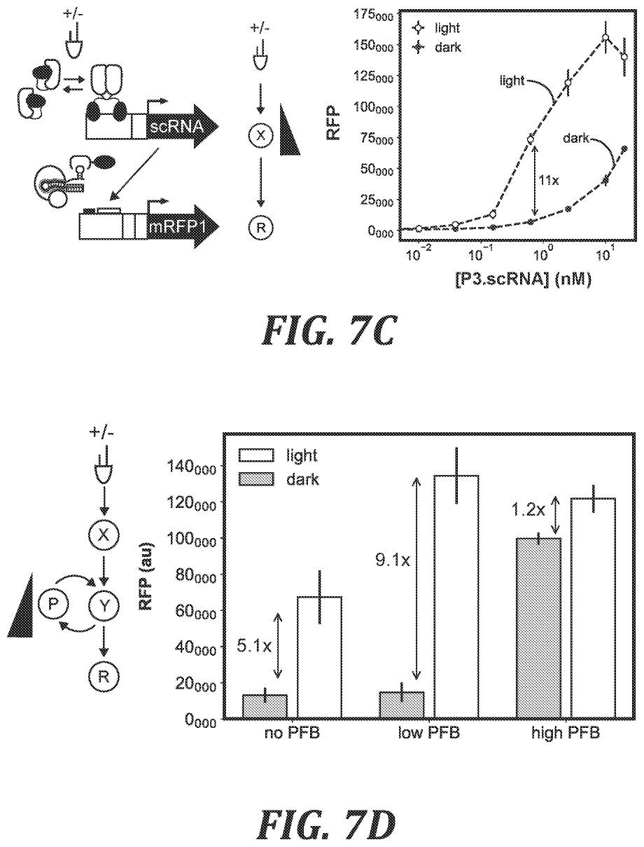

Having demonstrated that high-performing CRISPRa promoters can be generated through sequential screening, the same approach was tested for engineering activatable promoters responsive to other transcriptional activators. The EL222 transcriptional activator includes interactions with the RNAP and the DNA binding site and is a suitable model system for developing optogenetic inputs for CRISPRa/i circuits (FIG. 7A). Briefly, EL222 binds an 18 bp sequence upstream of the 35 region of the luxI promoter and subsequently recruits RNAP through interactions with the α and σ subunits. The luxI minimal promoter was mutagenized (FIG. 7A, Example 1 Materials and Methods), and variants screened in E. coli in dark and light to select for high dynamic range (FIG. 7B). Starting with a dynamic range of less than 2-fold, up to 4-fold dynamic range was observed in response to blue light. Similar to the CRISPRa promoter screens, minimal promoters with very low (BBa_J23113) or very high (BBa_J23119) basal expressions exhibited low dynamic range in response to blue light.

Four variants with >2-fold higher dynamic range than the luxI minimal promoter were selected and characterized in CFS. These variants yielded a 34.1-fold difference in expression between light and dark, compared to 6.2-fold for the original luxI minimal promoter (FIG. 10N). This improvement comes largely from a reduction in the basal expression from the blue light promoter, suggesting the RNAP-minimal promoter interactions were successfully minimized without weakening EL222-promoter interactions. More importantly, these results demonstrate that the disclosed approaches for engineering actionable promoters are applicable to other transcriptional activation systems.

### Blue Light Responsive CRISPRa/i Circuits

It was evaluated whether the engineered blue light promoter transcription levels were suitable for expressing gRNAs for CRISPRa/i circuits. The gRNA-expressing plasmid concentrations were titrated and RFP expression compared across dark and light conditions. For CRISPRi, the highest light-dependent change in repression was 50% (FIG. 10O). For CRISPRa, the highest light-dependent fold-activation was 11-fold (FIG. 7C), which improved to 14-fold upon increasing EL222 plasmid concentration (FIG. 10P).

A light-dependent activation cascade was constructed and only a 5-fold activation in response to blue light was observed (FIG. 7D). To improve the fold-activation, a positive feedback loop (PFB) in which a downstream node expresses a scRNA directing CRISPRa to an upstream node was implemented. The degree of positive feedback in the system can be tunable by titrating the PFB node, with high concentrations of this node resulting in activation in the absence of blue light. When optimally tuned, the PFB loop increases the light-dependent CRISPRa output levels almost 2-fold (FIG. 7D). Excess PFB node led to a 7.6-fold increase in basal expression, decreasing the light-dependent activation to 1.2-fold. These results highlight that rationally designed genetic circuits built from engineered activatable promoters can be used to improve the dynamic range of input-responsive signal processing modules.

### Engineered Activatable Promoters Enable Conditional CRISPRa Dependent On Protein-Protein Interactions

The versatility of protein-protein interaction (PPI)-mediated genetic regulation for coupling peptide or small-molecule binding to transcriptional outputs is of great interest to industry. In principle, CRISPRa assemblies incorporating dimerizing protein domains could be utilized for PPI-dependent transcriptional activation in bacterial CFS. However, realization of these systems has been limited by strict target site requirements and low dynamic range of activatable promoters. It was therefore reasoned that the high dynamic range promoters disclosed herein would allow to screen for otherwise hard-to-detect functional target sites and component stoichiometries and, if successful, achieve effective conditional, PPI-dependent CRISPRa.

### Development of Conditional CRISPRa Systems

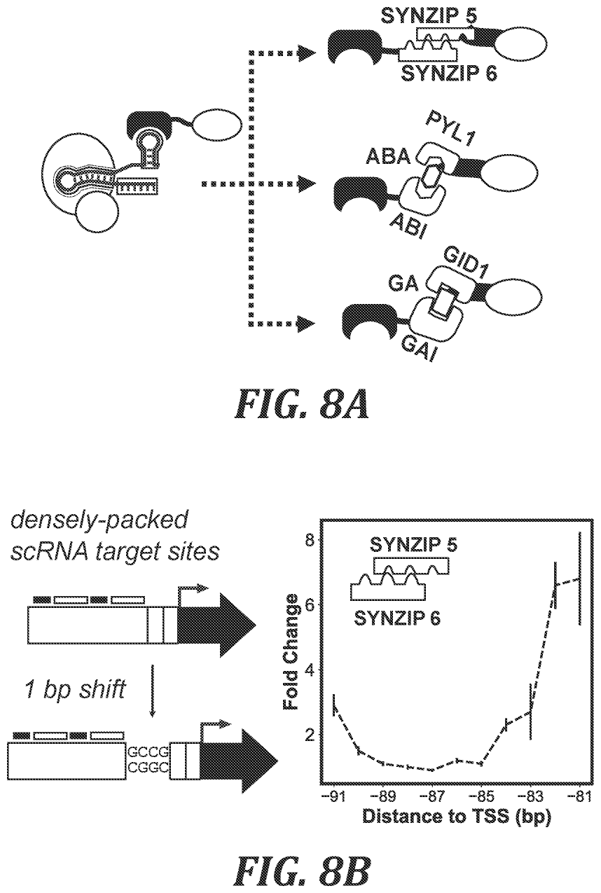

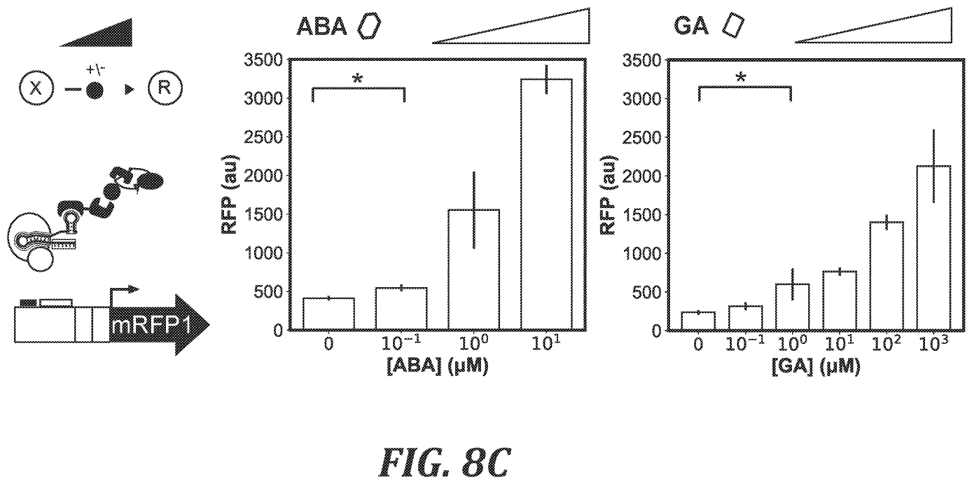

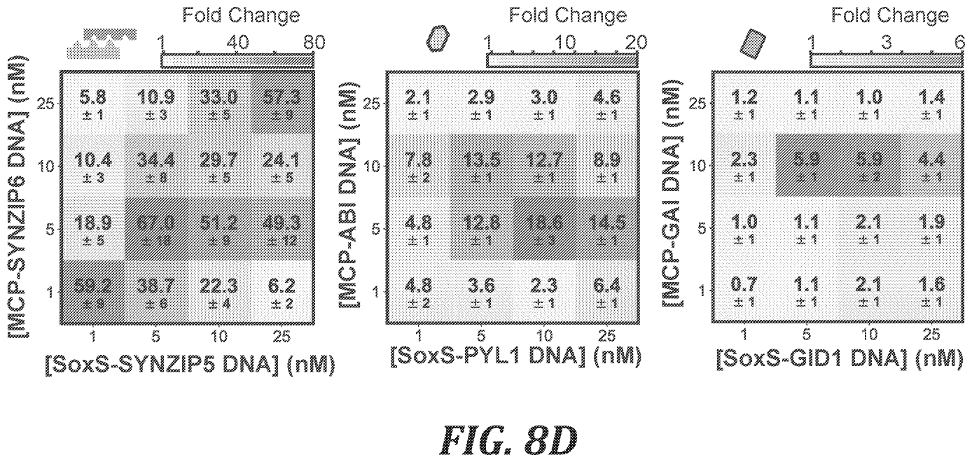

As experimental testbeds, three previously characterized protein-protein heterodimerization domains were incorporated into the CRISPRa system: the synthetic coiled-coil SYNZIP 5/6 pairs, the abscisic acid (ABA) responsive ABI-PYL1, and the gibberellic acid (GA) responsive GID1-GAI. These heterodimerization domains were fused to SoxS and MCP to enable conditional recruitment of SoxS to the CRISPRa complex. The MCP-SZP6 and SoxS-SZP5 domains were generated for SYNZIP-CRISPRa, the MCP-ABI and SoxS-PYL1 domains were generated for ABA-CRISPRa, and MCP-GAI and SoxS-GID1 were generated for GA-CRISPRa (FIG. 8A). In the original J3 promoter context, a 5.7-fold activation was observed when cognate SYNZIPs were fused to the C-termini of MCP (MCP-SYNZIP) and SoxS (SoxS-SYNZIP) (FIG. 10Q). This combination of orientations gave the best activation, compared to 2.8-fold when MCP was fused in the opposite orientation (SYNZIP-MCP & SoxS-SYNZIP), and 1.4-fold when SoxS was fused in the opposite orientation (MCP-SYNZIP & SYNZIP-SoxS) (FIG. 10Q). The fold-activation of ABA-CRISPRa was also maximized when SoxS was at the N-terminus (FIG. 10Q, right), therefore SoxS N-terminus fusions were used moving forward. For ABA- and GA-CRISPRa, only C-terminus MCP fusions were tested due in part to the MCP-SYNZIP result.

CRISPRa operates narrowly within −101:−71 bp from the TSS in a phase-dependent manner. It was tested whether the introduction of an additional protein linkage into MCP-SoxS affects the relative scRNA target site requirements. A CRISPRa promoter with densely-packed scRNA target sites every 10 bp was designed, as well as variants with 1 bp frameshifts to allow screening with single base pair resolution between −111:−81 bp from the TSS (FIG. 8B, Table 1). Surprisingly, SYNZIP-CRISPRa maintains the same preference for the targeting site-81 bp from the TSS and the same stringent 10-11 bp phase dependency seen in conventional CRISPRa (FIGS. 8B and 10R). SYNZIP-CRISPRa was then tested using a high-performing promoter with an optimal target site (HP3, Table 1) and a 5.4-fold improvement was found compared to the original J3 promoter (FIG. 10S).

To create functional systems for coupling peptide or small-molecule binding to CRISPRa-directed transcriptional outputs, permissible small-molecule input and component expression levels were identified. For ABA- and GA-CRISPRa, small-molecule titrations showed that ABA-CRISPRa is responsive between 0.1-10 uM with up to 7.9-fold-activation, and GA-CRISPRa is responsive between 1-1000 uM with up to 9.0-fold-activation (FIG. 8C). Dimer stoichiometries and expression levels were screened by surveying a range of concentrations for the MCP- and SoxS-fused components. SYNZIP-CRISPRa performs the best of the three systems, giving a maximal activation of 67-fold compared to a control without MCP or SoxS. Even at low concentrations of MCP- and SoxS-fused components, SYNZIP-CRISPRa still achieves 59-fold activation (FIG. 8D, left). ABA-CRISPRa gave a maximum activation of 18.6-fold (FIG. 8D, middle). For GA-CRISPRa, the maximum activation of 5.9-fold was accessible in a relatively narrow range of component concentrations (FIG. 8D, right). For all three PPI-dependent CRISPRa systems, higher expression of the MCP- and SoxS-fused heterodimer components did not necessarily improve activation. A unique optimal concentration for each PPI-dependent CRISPRa system was observed. Without wishing to be bound by any particular theory, the differences between systems may be due to the different affinities of each protein-protein interaction.

### Engineering Multi-Input CRISPRa/i Circuits

Multi-Layer and Multi-Input Circuits with Conditional CRISPRa

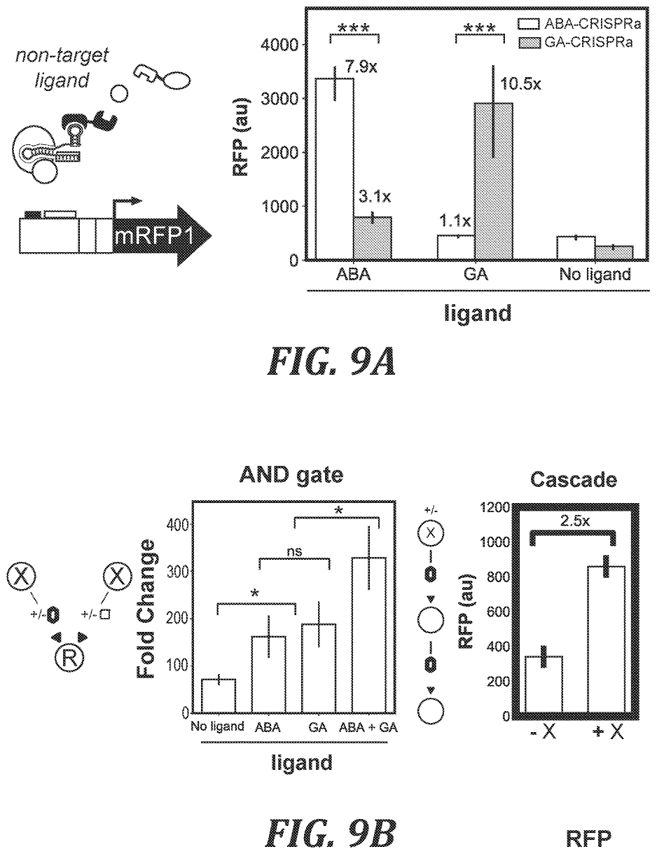

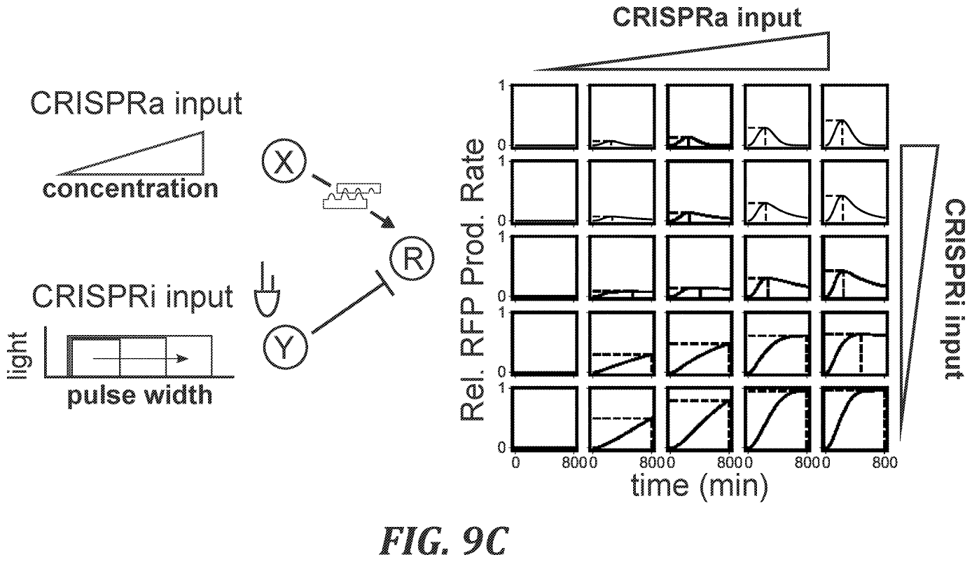

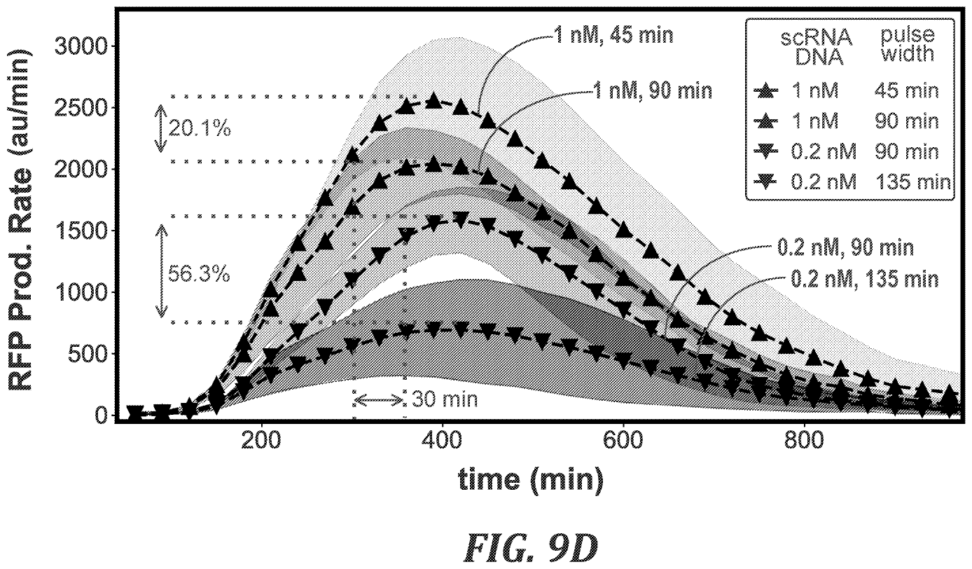

Two types of input-responsive circuits were built to explore the use of conditional CRISPRa for multi-input and multi-layer input processing: an AND-like logic gate and a CRISPRa cascade. A first step was characterizing the scRNA dose-response curve of the novel CRISPRa systems. For both conditional CRISPRa systems, the amount of scRNA needed to saturate the CRISPRa response was similar to that of direct CRISPRa (FIGS. 10G and 10T). The orthogonality of the small molecule systems was tested to evaluate if they could be used together for independent gene regulation (FIG. 9A). ABA-CRISPRa is highly specific to its target ligand, showing no significant activation in the presence of GA (p=0.21). GA-CRISPRa showed 3.1-fold cross-activation from ABA. Nonetheless, GA-CRISPRa maintained a 3-fold higher specificity for its cognate ligand, giving 10.5-fold activation from GA. These results suggested that the ABA- and GA-CRISPRa systems could be used for orthogonal gene regulation.

A multi-input circuit was constructed for AND logic by co-expressing components for ABA- and GA-CRISPRa (FIG. 9B, left). The addition of either ABA or GA resulted in 2-fold activation compared to the no-ligand condition (p=0.03). Therefore, it was specified that the average of the technical replicates must be above or below 2-fold activation for the circuit to be considered ON or OFF. Consistent with AND-like logic, the circuit generated 4.5-fold activation in the presence of both ligands, a level of activation that was clearly distinct from either of the one-input states (p=0.03). A multi-layer conditional CRISPRa cascade was built by having both internal layers dependent on ABA. The cascade gave 2.5-fold activation upon addition of ABA, showing that conditional CRISPRa can also support multi-layer information processing (FIG. 9B, right).

### Two-Input Dynamic Pulse Generator

Aspects of the disclosure relate to the creation of complex, dynamic signaling networks with multiple input-responsive regulators to tightly program the expression timing and magnitude of downstream targets. As an example, it was aimed to engineer a GRN that integrates blue light CRISPRi with PPI-dependent CRISPRa. The result is a tunable pulse generator with two-input control over the level and timing of gene expression (FIG. 9C, left). Because SYNZIP-CRISPRa has high-fold activation (FIG. 10T), it can be well suited for integration with blue light CRISPRi.

Simulation analysis was used to identify system designs for combining two inputs and dynamically regulating reporter gene expression. A coarse-grained mechanistic model of CRISPRa/i regulation was built upon by introducing blue light pulses regulating gRNA expression (Example 1 Materials and Methods). Changes in the pulse width were simulated as CRISPRi inputs and scRNA concentration as CRISPRa inputs. By evaluating changes in reporter production rates, the analysis suggested that there are broad ranges of CRISPRi and CRISPRa input parameter values that are compatible with multi-input-responsive regulatory control (FIG. 9C, left).

The results of the simulation analysis were then experimentally evaluated. To dynamically tune CRISPRi, the blue light exposure time was changed. To tune SYNZIP-CRISPRa, the scRNA-expressing plasmid concentration was changed. In all cases, the CFS was first kept in the dark for one hour to allow for EL222 expression. The RFP production rate pulse was tunable by the scRNA plasmid concentration and the blue light exposure time (FIG. 9D). When compared across conditions with the same CRISPRa input, higher CRISPRi input led to 20-56% lower maximum production rates. In conditions with the same CRISPRi input, higher CRISPRa input increased maximum production rates by 20-40 minutes. Collectively, these results highlight the potential of input-responsive CRISPRa/i GRNs for complex signal processing applications.

### DISCUSSION

In this example, it is shown that a trade-off between basal and activated expression levels can be relaxed to generate activatable promoters with both lower basal and higher activated expression levels than previously possible (FIGS. 4B and 5B). Sequential screening of promoter regions enabled to overcome context effects and identify high-performing activatable promoters (FIG. 5D). With this approach, orthogonal CRISPRa promoters that match the basal and activated expression levels of the canonical Tet inducible system (FIG. 10U) and exceed those of the IPTG inducible system were successfully engineered.

The E. coli transcriptional network is governed by a hierarchical structure containing nine layers of regulation. Engineered activatable promoters enabled to build multi-layer CRISPRa/i GRNs in E. coli-based CFS with depths and widths significantly larger than the state of the art. Specifically, a 33-fold improvement in promoter dynamic range resulted in 80% lower signal degradation in two-layer cascades, and enabled deep GRNs with up to six layers of regulation (FIG. 6E). Additionally, it is demonstrated that wide GRNs can regulate up to four parallel cascades (FIG. 6F), indicating that the CRISPRa/i framework is well suited for the design of wide control circuits for parallel computing and multi-gene regulation. It is also demonstrated that these circuits can be implemented in E. coli by constructing three-layer activation cascades with the internal nodes expressed from either high or low copy number plasmids (FIG. 10V). The E. coli cascades maintained more than 3-fold activation regardless of the copy numbers of the internal node plasmids (FIG. 10V), demonstrating the high correspondence between CFS and in vivo component function. The disclosed CRISPRa cascades are the deepest known CRISPRa cascades in CFS and in vivo. Without wishing to be bound by any particular theory, further improvements in GRN complexity may be difficult due to resource constraints, such as upstream gRNAs outcompeting downstream gRNAs for dCas9 binding (FIGS. 10H and 10V). Strategies to dynamically regulate upstream gRNA expression, such as reversing CRISPRa complex binding or implementing negative autoregulation motifs, can be implemented to enable even larger GRNs.

Biological systems continuously monitor and process environmental signals by using signal transduction modules as inputs to complex GRNs. This example provides a general framework for optimizing transcriptional activation systems at the promoter level and integrating them into CRISPRa/i GRNs. Promoter engineering of the optogenetic EL222 system enabled high light-dependent dynamic ranges (FIGS. 7B and 10N), with relevant expression levels for downstream applications (FIGS. 7C and 100). Through inducible gRNA expression, input signal modulation is demonstrated with various GRN topologies, including positive feedback loops and CRISPRa/i cascades (FIG. 7D), as well as integration of different signal transduction modules into the same CRISPRa/i GRN (FIG. 9B). Overall, this example demonstrates, among other abilities, the ability to achieve more complex biocomputing functions, including multi-input AND and NOT gates for targeted therapeutics and next-generation biosensors, through multi-input CRISPRa/i GRNs.

Protein-protein interactions have been used to execute complex, input-responsive functions in eukaryotes, however, developing similar systems in prokaryotes has been difficult, and the development of high dynamic range promoters as disclosed herein enabled to successfully prototype and optimize conditional CRISPRa systems in E. coli-based CFS. Implementation of novel conditional CRISPRa systems can be streamlined by the fact that all systems tested here are effective when targeted-81 bp from the TSS (FIGS. 8B and 10R), despite the presence of additional protein-protein interactions up to 500 amino acids in length. Additionally, conditional CRISPRa fold-activation is proportional to the strength of the protein-protein interaction (Table 2), which can be used to inform the α priori selection of heterodimers for use in conditional CRISPRa. Collectively, this work suggests that other heterodimerization domains could be implemented, with minimal prototyping, as signal transduction modules for CRISPRa/i GRNs for multiplexed biosensing or screening of PPIs in CFS.

The disclosed workflow for activatable promoter engineering enables the dynamic specification of expression levels for large networks of orthogonal gene targets. The new classes of deep, wide, and input-responsive CRISPRa/i GRNs disclosed herein have immediate application in CFS for investigating the rules of genetic circuit design and biological information processing, as well as for building dynamic, multi-enzyme expression programs for self-assembling bioproduction platforms. Moreover, CRISPRa/i GRNs can be integrated with existing field-deployable medical diagnostics and environmental monitors to enable complex, multi-input signal processing. Moving forward, this work can serve as a stepping stone for building entirely synthetic cells and engineered living materials with GRNs that match or go beyond the complexity of existing systems.

### Materials and Methods

### Design of Promoter Region Libraries

### Minimal Promoter Libraries

Transcription requires RNA polymerase (RNAP), a holoenzyme that includes five subunits (β, β′, α1, α2, ω) and a specificity factor (σ). In growing E. coli cells, most promoters are regulated by the transcription factor σ⁷⁰, which binds the −10 (⁻¹²TATAAT⁻⁷) and −35 (⁻³⁵TTGACA⁻³⁰) hexamers of a minimal promoter and enables transcription initiation. θ⁷⁰ binding is also influenced by the GC-content, the length, and the ⁻¹⁵TGn⁻¹ motif of the intervening sequence between the −10 and −35 sites. Two minimal promoter libraries were designed by mutagenizing the −10/−35 hexamers and the intervening sequence of the best-performing minimal promoter (BBa_J23117) (Table 3). The first library was designed by rationally mutagenizing specific bases that are known contacts of RNAP within the minimal promoter. The second library was made by randomly mutagenizing within the intervening sequence. Since the libraries yielded similar Pareto fronts, these mutations were then combined into a third minimal promoter library, which was used in the sequential screening process.

### UP-Element Libraries

Promoter recognition is enhanced by the AT-rich UP-elements upstream of the minimal promoter (distal ⁻⁶⁰NNNNAWWWWWTTTTT⁻⁴⁵ and proximal UP-element ⁻⁴⁵AAAAAARNR⁻³⁵), which anchor the α-subunits of RNAP. Various transcriptional activator proteins, including SoxS, also bind in this region to recruit RNAP through protein-protein interactions with the α-CTD. For effective CRISPRa, RNAP should only be recruited to the promoter in the presence of an on-target scRNA. Five UP-element libraries were designed by mutagenizing the AT-rich E. coli consensus sequence with increasing GC-content. Five libraries with 0%-100% GC-content, and a library representing the E. coli consensus sequence, were generated (Table 3).

scRNA Target Site Libraries

In the disclosed CRISPRa system, nuclease-defective S. pyogenes dCas9 is combined with scaffold RNAs (scRNAs) that specify DNA targets. Targeting of this complex to an appropriate site upstream of a minimal promoter results in CRISPRa. The sequence composition of the scRNA target site has an impact on basal expression levels. Three scRNA target site libraries with varying compositions of GC-content (0%, 50%, and 100%) were generated (Table 3). These libraries were used in tandem with a GC-rich UP-element.

### Cell-Free Reactions

### Cell-Free System Preparation

The cell-free system was acquired from Arbor Biosciences (myTXTL). The cell-free system used for an experiment was thawed on ice and pooled into a 1.5 mL Eppendorf tube, vortexed, and spun-down using a mini benchtop centrifuge to ensure homogeneity across samples.

### Cell-Free Gene Expression Reaction

Cell-free gene expression reactions were assembled on ice from the CFS and purified DNA. A master mix with common plasmids across reactions was prepared, and 1.5 μL per reaction allocated into PCR tubes. Plasmids which were varied across reactions were added in the remaining 1 μL. For reactions containing ABA (Sigma, A4906) or GA, 0.1 μL of the small molecules were added alongside the plasmids. For reactions involving more than 5 plasmids, plasmids were mixed with an acoustic liquid handler robot (Echo Labcyte 525). The CFS was pipette mixed and added to each PCR tube in 7.5 μL for a final volume of 10 μL. PCR tubes were vortexed, spun-down using a mini benchtop centrifuge, and placed on ice. Triplicates of 2.5 μL for each reaction were pipetted into individual wells of a 96-well V-bottom plate (Costar). The plate was sealed (Costar, Cat. 3080) and analyzed on a BioTek Synergy HTX plate reader at 29 °C mRFP1 fluorescence (ex. 540 nm, em. 600 nm) of cell-free reactions were measured every 10 min from the bottom of the plate. All reactions were run in batch mode.

### Optogenetic Experiments

For both E. coli and CFS optogenetic experiments, the cultures and reactions were prepared as described above. The incubation conditions were modified to include a blue light illumination source (UVP Visi-Blue UV Transilluminator, 8 Watts, 460/470 nm). The samples were placed at 37 °C or 29 °C in an incubator (Thermo Forma Orbital Shaker, Model #435) with the illumination source placed atop the incubator and irradiating inwards. E. coli cultures were covered with breathable membrane and left shaking overnight at 1200 RPM on a Heidolph Titramax 1000 inside the incubator at 37 °C. The distance between the illumination source and the deepwell plates was 14 cm. CFS reactions were placed inside the incubator at 29 °C at a distance of 6 cm with the bottom of the wells facing the illumination source. In both cases, the dark conditions were kept inside a cardboard box inside the incubator. Endpoint plate reader measurements were conducted using a BioTek Synergy HTX.

### Constitutive CRISPRa Experiments:

Endpoint CRISPRa experiments were conducted using constitutive expression of all CRISPRa components (pCK389). Circuits were assembled by transformation of CRISPRa and reporter plasmids into chemically competent MG1655 E. coli. Transformed E. coli were outgrown for 1 hour shaking at 37 °C and plated onto LB-agar with carbenicillin and chloramphenicol. Plates were grown overnight at 37 °C. Experiments were conducted by picking three individual colonies into 400 μL Teknova EZ-RDM with 0.2% glucose and appropriate antibiotics in 96 well plates (round 2 ml), covering with breathable membrane (Breathe Easier) and shaking overnight at 37 °C at 1200 RPM on a Heidolph Titramax 1000.
   aTc-Inducible Experiments

For inducible CRISPRa experiments expression of activator protein MCP-SoxS was controlled by pTet. Upon addition of aTc to media, pTet becomes de-repressed which enables titratable expression of MCP-SoxS activator. As above, circuits are assembled by transformation of CRISPRa and reporter plasmids into chemically competent MG1655 E. coli.

Transformed E. coli were outgrown for 1 hour shaking at 37 °C and plated onto LB-agar with carbenicillin and chloramphenicol. Plates were grown overnight at 37 °C. Experiments were conducted by picking individual colonies into 400 μL EZ-RDM with 0.2% glucose and appropriate antibiotics in 96 well plates (2 mL). Cultures are covered with breathable membrane and left shaking overnight at 37 °C at 1200 RPM on a Heidolph Titramax 1000. Overnight cultures are subsequently diluted 1:40 into a fresh plate of EZ-RDM and supplemented with appropriate concentrations of aTc. Cultures are again covered with a breathable membrane, incubated in the dark at 37 °C shaking at 1200 RPM, and grown overnight ~18 hours. Measurements are conducted in Costar 96 well black flat bottom plates in 100 μL culture volume.

### Plasmid Preparation for Cell-Free System

Plasmids intended for use in CFS were grown in culture volumes ~20 mL to ensure adequate yields for multiple cell-free reactions and were further purified using a PCR purification kit (Invitrogen PureLink, Cat. K310001), eluted into nuclease-free water. Plasmid concentrations were quantified via spectrophotometry (Nanodrop 2000c, Cat. ND-2000C).

### CFS Blue Light CRISPRa/i Modeling

The CFS blue light CRISPRa/i model constitutes a series of first order chemical reactions for protein and guide RNA production, CRISPR complex assembly, and DNA targeting and was implemented using the text-based model definition language Antimony for Python 3.7. Blue light regulation was introduced as a piecewise function that modulates transcription of the sgRNA required for CRISPRi. Linear functions of different slopes were used to capture the fast dimerization of the EL222 protein and binding to the DNA upon blight exposure as well as the slow unbinding in the absence of blue light. Specifically, the sgRNA basal transcription constant is modified with the following function:

$$0 if 0 < t < {t}_{delay}$$

$$\frac{{k}_{max}}{{t}_{O N}} ( t - {t}_{delay} ) if {t}_{delay} < t < {t}_{delay} + {t}_{O N}$$

$${k}_{max} if {t}_{delay} + {t}_{O N} < t < {t}_{delay} + {t}_{O N} + {t}_{expose}$$

$$\frac{- {k}_{max}}{{t}_{O F F}} ( t - ( {t}_{delay} + {t}_{O N} + {t}_{expose} ) if {t}_{delay} + {t}_{O N} + {t}_{expose} < t < {t}_{delay} + {t}_{O N} + {t}_{expose} + {t}_{OFF}$$

Where kₘₐₓ represents the transcription rate constant when EL222 is fully bound to the promoter, and t_{delay}, t_{ON}, tₑₓₚₒₛₑ, and t_{OFF} represent the time delay for light exposure, the time for EL222 dimerization and binding, the exposure time to light, and the time for EL222 unbinding upon removing the light source, respectively.

### Quantification and Statistical Analysis

E. coli data analysis:

Dynamic range:

Dynamic range was calculated as the ratio of measured RFP outputs without induction (0 nM aTc, or dark) and with induction (200 nM aTc, or light):

$$DR = \frac{{B}^{{α}_{1}} - {B}^{0}}{{B}^{{α}_{2}} - {B}^{0}}$$

where:
B is RFP/OD₆₀₀ measured at endpoint;
α₁ is activated expression, with induction;
α₂ is basal expression, without induction; and
0 is no RFP expression.

### Pareto Optimality:

To identify the best-performing promoter variants belonging to the Pareto front, the basal and activated RFP expression levels of each variant were compared to all other variants. A variant belongs to the Pareto front if no other variant had both lower basal and higher activated expression levels:
vₒ ∈P(V) if there is no v such that

$$( {v}^{a} > {v}_{o}^{a} \& {v}^{b} < {v}_{o}^{b} )$$

### for all v ∈V

where:
V is the set of all promoter variants;
vₒ is a variant in V;

$${v}_{o}^{a} , {v}_{o}^{b}$$

are the activated and basal expression levels of said variant; and
P(V) is the set of promoter variants belonging to the Pareto front.

### Cell-Free Data Analysis

### Production Rate:

Throughout this example, production rate is defined as:

$${\dot{B}}^{α} ( t ) = \frac{d {B}^{α}}{dt} = \frac{{B}^{α} ( t + 30 ) - {B}^{α} ( t )}{30}$$

where:
B is the measured RFP; and
α specifies the circuit topology and relevant plasmid concentrations.

### Relative Production Rates:

Relative production rates of CRISPRa mediated outputs were calculated as the ratio of CRISPRa mediated production rates divided by unregulated production rates. For CRISPRa the contribution due to unregulated basal expression was subtracted from measured output levels due to CRISPRa. This was done to isolate the timing of CRISPRa mediated gene expression from the comparatively early contribution of basal expression, and to allow observation of CRISPRa mediated gene expression dynamics under conditions where basal expression of reporter constructs dominates. Throughout this example, relative production rates are abbreviated as Rel. RFP Prod. Rate and are calculated as:

$${\dot{B}}_{Γ}^{α} ( t ) = \frac{{\dot{B}}^{α} ( t ) - {\dot{B}}^{Γ} ( t )}{{\dot{B}}^{Γ} ( t )}$$

where:
α is a specific CRISPRa/i circuit; and
Γ is constitutive expression.

### Fold Change:

Fold change was calculated as the ratio of RFP values generated by CRISPRa in the presence of input scRNA compared to RFP values generated in the absence of input scRNA.

$${FC}^{α} ( y ) = \frac{{B}^{α +} ( t ) - {B}^{Γ} ( t )}{{B}^{α -} ( t ) - {B}^{Γ} ( t )}$$

where:
α+ is CRISPRa with y nM input scRNA;
α− is CRISPRa without input scRNA; and
Γ is constitutive expression.

### Time to Maximum Expression Rate:

To calculate the time to maximum expression rate, the contribution due to unregulated basal expression was subtracted from measured RFP levels due to CRISPRa. This was done to isolate the timing of CRISPRa mediated gene expression from the comparatively early contribution of leak, and to allow observation of CRISPRa mediated gene expression dynamics under conditions where basal expression of reporter constructs dominates. The time to maximum expression is denoted as tₘₐₓ.

$$t = {t}_{max} when {\dot{B}}_{Γ}^{α} ( t ) = max ( {\dot{B}}_{Γ}^{α} ( t ) )$$

where:
α is a specific CRISPRa/i circuit; and
Γ is constitutive expression.

Change in time to maximum expression rate (Δtₘₐₓ) is calculated by finding the difference in time to reach maximum production rate between the with and without input conditions.

$$Δ {t}_{max} = {t}_{max}^{α +} - {t}_{max}^{α -}$$

### Signal Propagation Efficiency:

Propagation efficiency of the CRISPRa cascade in CFS was calculated as the maximum fold change in cascade output±input divided by the fold change provided by CRISPRa in the input layer at the same time point.

$$propagation = 100 \cdot \frac{max ( F {C}^{α} ( y ) )}{F {C}^{β} ( y )}$$

where:
α is CRISPRa cascade with y nM of scRNAs; and
β is CRISPRa with y nM of scRNAs.

### Signal Delay:

Signal delay is calculated as the difference in time to reach the maximum fold change of the cascade between the cascade output and input layer.

$${t}^{α} = {t}_{max}^{α} FC when {FC}^{α} ( y ) = max ( F {C}^{α} ( y ) )$$

$${t}^{β} = {t}_{max}^{β} FC when {FC}^{β} ( y ) = max ( F {C}^{α} ( y ) )$$

$$delay = {t}_{max}^{β} F C - {t}_{max}^{α} F C$$

where:
α is CRISPRa cascade with y nM of scRNAs; and
β is CRISPRa with y nM of scRNAs.

### Relationship Between Signal Delay and Signal Propagation

Fraction of signal propagation at the nth node is defined to be the product of the fraction of signal propagated at the previous nodes, namely:

$${fSP}_{n} = {\prod }_{i}^{n} {fSP}_{i}$$

where the fraction of signal propagated by each node i is a function of the characteristic relative fold-change of node i, as well as the time of the reaction at which the signal propagates through node i:

$${fSP}_{i} = {rFC}_{i} \cdot {e}^{\frac{- ( {θ}_{i} + {t}_{o} )}{τ}}$$

where tₒ is the reaction boot up time, and τ is the characteristic time of the system. While seemingly simple, the exponential term accounts for the complex dynamics of cell-free expression and gRNA competition, and favors expression from earlier nodes.

The time of the reaction at which the signal propagates through the nth node can be estimated based on the fraction of the signal propagated through the nth node and the relative lifetime of the reaction:

θₙ=rLT·(1−fSPₙ)
where the relative lifetime of the reaction is the difference between the time to maximum fold activation of a one-layer cascade and the end of the reaction. With these equations and the characteristic relative fold-change of each node, both the signal delay and probation can be calculated iteratively.

Based on kinetic data, t₀ and rLT are set to be ~2 hrs and ~5 hrs, respectively. In order to estimate τ, the model is fit to empirical signal propagation and delay data by minimizing the sum of residuals using the Nelder-Mead algorithm.

### Cell-Free Gene Expression Reaction

Cell-free gene expression reactions were assembled on ice from the CFS and purified DNA. A master mix with common plasmids across reactions was prepared, and 1.5 μL per reaction allocated into PCR tubes. Plasmids which were varied across reactions were added in the remaining 1 μL. For reactions containing ABA (Sigma, A4906) or GA, 0.1 μL of the small molecules were added alongside the plasmids. For reactions involving more than 5 plasmids, plasmids were mixed with an acoustic liquid handler robot (Echo Labcyte 525). The CFS was pipette mixed and added to each PCR tube in 7.5 μL for a final volume of 10 μL. PCR tubes were vortexed, spun-down using a mini benchtop centrifuge, and placed on ice. Triplicates of 2.5 μL for each reaction were pipetted into individual wells of a 96-well V-bottom plate (Costar, Cat. 3363). The plate was sealed (Costar, Cat. 3080) and analyzed on a BioTek Synergy HTX plate reader at 29 °C mRFP1 fluorescence (ex. 540 nm, em. 600 nm) of cell-free reactions were measured every 10 min from the bottom of the plate. All reactions were run in batch mode.

### Plasmid and Library Construction

All PCR amplification of plasmids and fragments used Phusion DNA polymerase in GC buffer. Primers were synthesized by IDT and resuspended into nuclease-free water. All PCR reactions were treated with DpnI for longer than 1 hour and purified using Qiagen gel extraction kits. Plasmid assembly was achieved using 5× In-Fusion HD mastermix (Takara).

Assembled plasmids and libraries were transformed into chemically competent NEB Turbo E. coli and plated onto LB-agar plates with either 100 μg/mL carbenicillin or 25 μg/mL chloramphenicol. Transformed cells were grown overnight ~16 hours at 37 °C. Single colonies were picked from plates and grown overnight in LB shaking at 37 °C with appropriate concentrations of relevant antibiotics.

E. coli Experiments Culturing and Quantification Conditions

Transformed E. coli were outgrown for 1 hour shaking at 37 °C and plated onto LB-agar with carbenicillin and chloramphenicol. Plates were grown overnight at 37 °C. Experiments were conducted by picking three individual colonies into 400 μL Teknova EZ-RDM with 0.2% glucose and appropriate antibiotics in 96 well plates, covering with breathable membrane (Breathe Easier cat #Z763624) and shaking overnight at 37 °C at 1200 RPM on a Heidolph Titramax 1000. For inducible experiments, overnight cultures are subsequently diluted 1:40 into a fresh plate of EZ-RDM and supplemented with appropriate concentrations of aTc. Plate reader measurements were conducted using a BioTek Synergy HTX with a black flat bottom plate (Ref #3631) using 100 μL of culture.

### Plasmid and Library Preparation

Details regarding plasmid and library construction are presented herein. Plasmids were transformed into chemically competent NEB Turbo E. coli. 10 μL of the outgrowth with transformed libraries was diluted 1:20 with LB and plated onto LB-agar with carbenicillin to check library complexity. The remaining outgrowth was seeded into 5 mL of LB with carbenicillin or gentamicin. Cells were grown overnight ~16 hours at 37 °C. Single colonies were picked from plates and grown overnight in LB with carbenicillin. Single colonies and culture were sequence verified. Plasmids were isolated from subcultures using a DNA miniprep kit (QIAprep Spin Miniprep Kit) and Sanger sequenced (Genewiz, Inc.).

E. coli Experiments

dCas9, MCP-SoxS, and scRNA are on a p15A ori plasmid while reporter construct is located on a pSC101** ori plasmid. For experiments involving more than two plasmids, competent cells were first made from cells carrying the reporter plasmid and the CRISPRa plasmid (including either on- or off-target input scRNAs). The appropriate plasmids expressing internal scRNAs were transformed into the competent cells. Details regarding culturing conditions and quantification are provided herein.

Minimal Promoter Libraries: MP1 was designed by rationally mutagenizing specific bases that are known contacts of RNAP within the minimal promoter. MP2 was made by randomly mutagenizing within the intervening sequence. Since the libraries yielded similar Pareto fronts, these mutations were combined into MP3, and used in the sequential screening process (Table 3).

EL222 Minimal Promoter Libraries: Starting with the native luxI minimal promoter, rational mutations were introduced to make it resemble a synthetic activatable promoter (J23117). The −10:−35 region was then randomly mutagenized (Table 3).

### Cell-Free System Preparation

CFS was acquired from Arbor Biosciences (myTXTL). The CFS used for an experiment was thawed on ice and pooled into a 1.5 ml Eppendorf tube, vortexed, and spun-down using a mini benchtop centrifuge to ensure sample homogeneity. Details about plasmid preparation and details about the CFS reaction are provided herein.

Data analysis: Throughout this work all measured RFP levels in E. coli were normalized by measured OD600 with appropriate propagation of uncertainties. All metrics are described herein.

Statistics: Statistical significance was calculated using two-tailed unpaired Welch's t-tests. Asterisks in Figures indicate a statistically significant difference (*: p-value <0.05, **: p-value <0.01, ***: p-value <0.001).

TABLE 1
TABLES
Promoters generated.

| | | | Minimal | | |
|---|---|---|---|---|---|
| Promoter | scRNA target | UP-Element | Promoter | Basal | Activated |
| HP1_J3. | AACTCTCACACG | CCGGCGGCGG | TTGACAGTTT | 773.66 | 289908.0 |
| DA9.2A | TGGCTGCA | CGGCTGCCGC | TACGATGTGT | ± | ± |
| | (SEQ ID | GCGGCG | TGGGATTGTG | 123.99 | 22289.67 |
| | NO: 20) | (SEQ ID | CTAGC (SEQ | | |
| | | NO: 33) | ID NO: 50) | | |
| HP2_J3. | AACTCTCACACG | CCGCGGGCGG | TTGACACTTC | 690.33 | 351826.66 |
| DA9.1C | TGGCTGCA | CCGCTGCCGG | CGGCACGAAA | ± | ± |
| | (SEQ ID | GGCGCG | AGGGATTGTG | 112.43 | 7551.20 |
| | NO: 21) | (SEQ ID | CTAGC (SEQ | | |
| | | NO: 34) | ID NO: 51) | | |
| HP3_J3. | AACTCTCACACG | CCGCGGGCGG | TTGACGCCTC | 153.33 | 163114.33 |
| DA9.1B | TGGCTGCA | CCGCTGCCGG | CTTCTTTCTT | ± | ± |
| | (SEQ ID | GGCGCG | AGGGATTGTG | 43.81 | 10141.27 |
| | NO:2 2) | (SEQ ID | CTAGC (SEQ | | |
| | | NO: 35) | ID NO: 52) | | |
| J3.DA2.1B | CGCCGAATGCTC | CCGCGGGCGG | TTGACGCCTC | 567.33 | 380117.0 |
| | TAGCGGGA | CCGCTGCCGG | CTTCTTTCTT | ± | ± |
| | (SEQ ID NO: | GGCGCG | AGGGATTGTG | 20.21 | 73224.73 |
| | 23) | (SEQ ID | CTAGC (SEQ | | |
| | | NO: 36) | ID NO: 53) | | |
| J3.DA3.1B | CACACCTAAGTC | CCGCGGGCGG | TTGACGCCTC | 595.0 | 279047.33 |
| | AGGATTGT | CCGCTGCCGG | CTTCTTTCTT | ± | ± |
| | (SEQ ID NO: | GGCGCG | AGGGATTGTG | 39.05 | 14991.68 |
| | 24) | (SEQ ID | CTAGC (SEQ | | |
| | | NO: 37) | ID NO: 54) | | |
| J3.DA4.1B | CATATCTCTGAC | CCGCGGGCGG | TTGACGCCTC | 263.66 | 332795.33 |
| | CTGATCGA | CCGCTGCCGG | CTTCTTTCTT | ± | ± |
| | (SEQ ID NO: | GGCGCG | AGGGATTGTG | 9.18 | 7755.96 |
| | 25) | (SEQ ID | CTAGC (SEQ | | |
| | | NO: 38) | ID NO: 55) | | |
| J3.DA6.1B | CACATAAAAACC | CCGCGGGCGG | TTGACGCCTC | 370.66 | 313809.33 |
| | GCTGACTA | CCGCTGCCGG | CTTCTTTCTT | ± | ± |
| | (SEQ ID NO: | GGCGCG | AGGGATTGTG | 40.35 | 15988.97 |
| | 26) | (SEQ ID | CTAGC (SEQ | | |
| | | NO: 39) | ID NO: 56) | | |
| J3.DA9.1B | AACTCTCACACG | CCGCGGGCGG | TTGACGCCTC | 186.0 | 224187.33 |
| | TGGCTGCA | CCGCTGCCGG | CTTCTTTCTT | ± | ± |
| | (SEQ ID NO: | GGCGCG | AGGGATTGTG | 79.92 | 20739.19 |
| | 27) | (SEQ ID | CTAGC (SEQ | | |
| | | NO: 40) | ID NO: 57) | | |
| J3.DA10.1B | AGAAACAGTAAA | CCGCGGGCGG | TTGACGCCTC | 425.33 | 349136.66 |
| | AACTTTCA | CCGCTGCCGG | CTTCTTTCTT | ± | ± |
| | (SEQ ID NO: | GGCGCG | AGGGATTGTG | 29.86 | 15853.45 |
| | 28) | (SEQ ID | CTAGC (SEQ | | |
| | | NO: 41) | ID NO: 58) | | |
| EP1 | — | TCGGTAGCCT | TTGACGCTGT | 1053.66 | 10081.91 |
| | | TTAGTCCATG | ATTCAGGCAA | ± | ± |
| | | (SEQ ID | AGGGATTGTG | 82.92 | 940.10 |
| | | NO: 42) | CTAGC (SEQ | | |
| | | | ID NO: 59) | | |
| EP2 | — | TCGGTAGCCT | TTGACAGTGC | 6170.33 | 117299.0 |
| | | TTAGTCCATG | GTACGCAGGG | ± | ± |
| | | (SEQ ID | AGGGATTGTG | 113.42 | 5279.40 |
| | | NO: 43) | CTAGC (SEQ | | |
| | | | ID NO: 60) | | |
| EP3 | — | TCGGTAGCCT | TTGACGGTGA | 3516.66 | 120075.33 |
| | | TTAGTCCATG | AGAGTATCAG | ± | ± |
| | | (SEQ ID | AGGGATTGTG | 217.243 | 7740.25 |
| | | NO: 44) | CTAGC (SEQ | | |
| | | | ID NO: 61) | | |
| EP4 | — | TCGGTAGCCT | TTGACAGCTC | 5079.66 | 66449.16 |
| | | TTAGTCCATG | AGTGAGTAGT | ± | ± |
| | | (SEQ ID | AGGGATTGTG | 417.91 | 6693.89 |
| | | NO: 45) | CTAGC (SEQ | | |
| | | | ID NO: 62) | | |
| R2.E8.R206 | (−81) | CCGGCCCCCC | TTGACAAAGC | 250.67 | 6287.67 |
| | TCGGCTCACTTA | CCGCTGCCGC | TATGGCCGGC | ± | ± |
| | TGCACGGC | GGGCCG | AGGGATTGTC | 21.2 | 670.47 |
| | (SEQ ID | (SEQ ID | ACAGC (SEQ | | |
| | NO: 29) | NO: 46) | ID NO: 63) | | |
| R2.E8.R208 | (−91) | CCGGCCCCCC | TTGACAAAGC | 250.67 | 3056.67 |
| | TCCCTGACCCTC | CCGCTGCCGC | TATGGCCGGC | ± | ± |
| | GGCTCACT | GGGCCG | AGGGATTGTC | 21.2 | 414.77 |
| | (SEQ ID | (SEQ ID | ACAGC (SEQ | | |
| | NO: 30) | NO: 47) | ID NO: 64) | | |
| R2.E8.R210 | (−101) | CCGGCCCCCC | TTGACAAAGC | 250.67 | 394.67 |
| | ACCCTTTCCTTC | CCGCTGCCGC | TATGGCCGGC | ± | ± |
| | CCTGACCC | GGGCCG | AGGGATTGTC | 21.2 | 56.89 |
| | (SEQ ID | (SEQ ID | ACAGC (SEQ | | |
| | NO: 31) | NO: 48) | ID NO: 65) | | |
| R2.E8.R212 | (−111) | CCGGCCCCCC | TTGACAAAGC | 250.67 | 206.67 |
| | TTCCTCTCCTAC | CCGCTGCCGC | TATGGCCGGC | ± | ± |
| | CCTTTCCT | GGGCCG | AGGGATTGTC | 21.2 | 4.51 |
| | (SEQ ID NO: | (SEQ ID | ACAGC (SEQ | | |
| | 32) | NO: 49) | ID NO: 66) | | |

TABLE 2
Dimerization domain affinities.

| Domain | K_{d} | | Method of measurement |
|---|---|---|---|
| MS2:MCP | 0.0033 | μM | Filter binding assay |
| SYNZIP5: | <0.015 | μM | Fluorescence polarization |
| SYNZIP6 | | | |
| PYL1:ABA | 1-52 | μM | Isothermal titration |
| | | | calorimetry. |
| | | | Surface plasmon resonance |
| PYL1: ABA:ABI | 0.030 | μM | Isothermal titration |
| | | | calorimetry |
| GID1:GA | 0.2-4 | μM | Radioactivity assay with |
| | | | isotopically labeled GA, In |
| | | | vitro FRET binding assay, |
| | | | Surface plasmon resonance |
| GAI:GA:GID1 | 0.180 | μM | Surface plasmon resonance |

TABLE 3
Primers for promoter mutagenesis.

| Minimal Promoter | Sequence |
|---|---|
| oMP1 | CGATTATAGATtgacRgctagctcagtcct |
| | DgNNaYNGtgctagcGAATTCATTAAAGAG (SEQ ID |
| | NO: 67) |
| oMP2 | CGATTATAGAttgacttgacaNNNNNNNNN |
| | NNNNNagggattgtNNNAGCGAATTCATTA |
| | AAGAG (SEQ ID NO: 68) |
| oMP3_S2 | CGCTAGCACAATCCCWNNNNNNSNNNNMRS |
| | YGTCAAGCCGGGAGAGCTGGTTCCATTGCG |
| | ATTGCAGCCACGTGTGAGAGTT (SEQ ID NO: 69) |
| oMP3_S2 | CGCTAGCACAATCCCWNNNNNNSNNNNMRS |
| | YGTCAACGCCGCCCGGCAGCCGGCGCGGGC |
| | GCTGCAGCCACGTGTGAGAGTT (SEQ ID NO: 70) |
| oMP3 S2 | CGCTAGCACAATCCCWNNNNNNSNNNNMRS |
| | YGTCAACGCCGCGCGGCAGCCGCCGCCGCC |
| | GGTGCAGCCACGTGTGAGAGTT (SEQ ID NO: 71) |
| UP-element | Sequence |
| oUP1 | GctagctgtcaaYNYTTTTTTAAAAAWWWW |
| | WTNNnnnnTTGTGTCCAGAACGCTCCGTAG (SEQ ID |
| | NO: 72) |
| oUP2 | GctagctgtcaawwWWWWWWWWWWWWWWWW |
| | WWWWWWWWTTGTGTCCAGAACGCTCCGTAG (SEQ ID |
| | NO: 73) |
| oUP3 | Gctagctgtcaawwwhdwhdwhdwhdwwdw |
| | hwwhdwwwTTGTGTCCAGAACGCTCCGTAG (SEQ ID |
| | NO: 74) |
| oUP4 | GctagctgtcaaNNNNNNNNNnnnNNNNNN |
| | NNNNNNNNTTGTGTCCAGAACGCTCCGTAG (SEQ ID |
| | NO: 75) |
| oUP5 | GctagctgtcaaBBBBSSSSBbnnNNBSSS (SEQ ID |
| | BSSSBSSSTTGTGTCCAGAACGCTCCGTAG |
| | NO: 76) |
| oUP6 | GctagctgtcaaSSSSSSSSSsssSSSSSS (SEQ ID |
| | SSSSSSSSTTGTGTCCAGAACGCTCCGTAG |
| | NO: 77) |
| oUP6_S1 | GctagctgtcaaSSSSSSSSSSSSSSSSSS (SEQ ID |
| | SSSSSSSSTGCAGCCACGTGTGAGAGTTAG |
| | NO: 78) |
| scRNA target site | Sequence |
| oTS1 | GCTCGTCTCCTCACTTCTCCTWWWWWWWWW |
| | WWWWWWWWWWWCCGGCCCCCCCCGCTGCCG |
| | CGGGCCGttgacagctagctcagtcctagg (SEQ ID |
| | NO: 79) |
| oTS2 | GCTCGTCTCCTCACTTCTCCTNNNNNNNNN |
| | NNNNNNNNNNNCCGGCCCCCCCCGCTGCCG |
| | CGGGCCGttgacagctagctcagtcctagg (SEQ ID |
| | NO: 80) |
| oTS3 | GCTCGTCTCCTCACTTCTCCTSSSSSSSSS |
| | SSSSSSSSSSSCCGGCCCCCCCCGCTGCCG |
| | CGGGCCGttgacagctagctcagtcctagg (SEQ ID |
| | NO: 81) |
| EL222 promoter | Sequence |
| OMPE | CCTCTTTAATGAATTCGCTNNNACAATCCC |
| | WNNNNNNSNNNNMRSYGTCAACATGGACTA |
| | AAGGCTACCTATAAA (SEQ ID NO: 82) |

TABLE 4
Plasmids used.

| | J3 | scRNA | UP- | Promoter | | | | | | |
|---|---|---|---|---|---|---|---|---|---|---|
| Plasmid | Promoter | target | Element | Minimal | RBS | CDS | gRNA | Terminator | res* | ori** |
| pJF143.J3 | J3 | J306 | J3 | J23117 | Bujard | mRFP1 | X | dbl term | A | S |
| pCK389.gRNA | X | X | X | Sp.Cas9, | , Bujard, | Sp.Cas9, | J306, | Dbl term, | C | A |
| | | | | pTet, | X | MCP-SoxS | AAV, | BBa_B1002, | | |
| | | | | J23119 | | (R93A, | DA9 | TrrnB | | |
| | | | | | | S101A) | | | | |
| pJF182.gRNA | X | X | X | Sp.Cas9, | , Bujard, | Sp.Cas9, | J306, | dbl term, | C | A |
| | | | | J23107, | X | MCP-SoxS | AAV | BBa_B1002, | | |
| | | | | J23119 | | (R93A, | | TrrnB | | |
| | | | | | | S101A) | | | | |
| pDA010.188 | X | X | X | J23107 | Bujard | Sp. | X | ECK120033736 | A | E |
| | | | | | | dCas9 | | | | |
| pRC029 | X | X | X | J23107 | Bujard | MCP-SoxS | X | ECK120033736 | A | E |
| | | | | | | (R93A, | | | | |
| | | | | | | S101A) | | | | |
| pRC011 | X | X | X | J23107 | Bujard | MCP-SYNZIP6 | X | ECK120033736 | A | E |
| pRC012 | X | X | X | J23107 | Bujard | SoxS-SYNZIP5 | X | ECK120033736 | A | E |
| pRC025 | X | X | X | J23107 | Bujard | MCP-ABI | X | ECK120033736 | A | E |
| pRC027 | X | X | X | J23107 | Bujard | SoxS-PYL1 | X | ECK120033736 | A | E |
| pRC042 | X | X | X | J23107 | Bujard | MCP-GAI | X | ECK120033736 | A | E |
| pRC043 | X | X | X | J23107 | Bujard | SoxS-GID1 | X | ECK120033736 | A | E |
| pWS025.BLNNN-RFP | X | X | X, | J23119, N | BBa_B0034, | EL222, | X | BBa_B0015_dblT, | A | S |
| | | | EL222_Binding_region | | Bujard | mRFP1 | | dbl term | | |
| pDA010.EL222 | X | X | X | J23106 | BBa_B0034 | EL222 | X | BBa_B0015_dblT | A | E |
| pDA040.BLD7-mRFP | X | X | EL222_Binding_region | D7 | X | mRFP1 | X | ECK120033736 | A | E |
| pDA040.BLD7-DA9 | X | X | EL222_Binding_region | D7 | X | X | DA9 | ECK120033736 | A | E |
| pDA040.BLD7-RR2 | X | X | EL222_Binding_region | D7 | X | X | RR2 | ECK120033736 | A | E |
| pDA303 | J3 | DA9 | 1 | B | Bujard | mRFP1 | X | ECK120033736 | A | E |
| pDA304 | J3 | DA2 | 1 | B | Bujard | mRFP1 | X | ECK120033736 | A | E |
| pDA305 | J3 | DA3 | 1 | B | Bujard | mRFP1 | X | ECK120033736 | A | E |
| pDA306 | J3 | DA4 | 1 | B | Bujard | mRFP1 | X | ECK120033736 | A | E |
| pDA307 | J3 | DA6 | 1 | B | Bujard | mnRFP1 | X | ECK120033736 | A | E |
| pDA309 | J3 | DA10 | 1 | B | Bujard | mRFP1 | X | ECK120033736 | A | E |
| pDA310 | J3 | DA9 | 1 | B | X | X | DA2 | ECK120033736 | A | E |
| pDA311 | J3 | DA9 | 1 | B | X | X | DA3 | ECK120033736 | A | E |
| pDA312 | J3 | DA9 | 1 | B | X | X | DA4 | ECK120033736 | A | E |
| pDA313 | J3 | DA9 | 1 | B | X | X | DA6 | ECK120033736 | A | E |
| pDA314 | J3 | DA9 | 1 | B | X | X | DA8 | ECK120033736 | A | E |
| pDA315 | J3 | DA2 | 1 | B | X | X | DA10 | ECK120033736 | A | E |
| pDA316 | J3 | DA3 | 1 | B | X | X | DA10 | ECK120033736 | A | E |
| pDA317 | J3 | DA4 | 1 | B | X | X | DA10 | ECK120033736 | A | E |
| pDA318 | J3 | DA6 | 1 | B | X | X | DA10 | ECK120033736 | A | E |
| pDA319 | J3 | DA8 | 1 | B | X | X | DA10 | ECK120033736 | A | E |
| pDA320 | J3 | DA9 | 1 | B | X | X | DA2 | ECK120033736 | A | E |
| pDA321 | J3 | DA2 | 1 | B | X | X | DA3 | ECK120033736 | A | E |
| pDA322 | J3 | DA3 | 1 | B | X | X | DA4 | ECK120033736 | A | E |
| pDA323 | J3 | DA4 | 1 | B | X | X | DA6 | ECK120033736 | A | E |
| pDA324 | J3 | DA6 | 1 | B | X | X | DA8 | ECK120033736 | A | E |
| pDA325 | J3 | DA8 | 1 | B | X | X | DA10 | ECK120033736 | A | E |
| pRC014.(0-4) | R2 | R206 (−81), | E | 8 | Bujard | mRFP | X | ECK120033736 | A | E |
| | | R208 (−91), | | | | | | | | |
| | | R210 (−101), | | | | | | | | |
| | | R212 (−111) | | | | | | | | |
| pCK956.gRNA | X | X | X | J23107, | Bujard, | Sp.Cas9, | J106, | Dbl term, | C | A |
| | | | | J23107, | Bujard, | MCP-SoxS | AAV | BBa_B1002, | | |
| | | | | pTet | X | (R93A, | | TrrnB | | |
| | | | | | | S101A) | | | | |
| pDA506.gRNA | X | gRNA | 1 | B | Bujard | sfGFP | X | dbl term | K | S |
| pCK957 | X | J106 | 1 | B | X | X | DA4 | Sht TermA | C, S | E, D |
| pCK958 | X | J106, DA4 | 1 | B | X | X | DA4, DA9 | Sht TermA | C, S | E, D |
| pCK960 | X | DA4 | 1 | B | X | X | DA9 | Sht TermA | C, S | E, D |

*Resistance marker: C stands for chloramphenicol, A stands for ampicillin, S stands for spectinomycin, K stands for kanamycin.
**Origin of replication: E stands for ColE1, A stands for p15A, and S stands for sc101** , and D stands for CloDF13.

TABLE 5
Deep cascade concentrations (all plasmid concentrations are in nM).

| Cascade/Plasmid | D 1− | D 1+ | D 2− | D 2+ | D 3− | D 3+ | D 4− | D 4+ | D 5− | D 5+ | D 6− | D 6+ |
|---|---|---|---|---|---|---|---|---|---|---|---|---|
| pDA010.188 | 4 | 4 | 4 | 4 | 4 | 4 | 4 | 4 | 4 | 4 | 4 | 4 |
| pRC029 | 4 | 4 | 4 | 4 | 4 | 4 | 4 | 4 | 4 | 4 | 4 | 4 |
| pBT009.J1.119.DA4 | 0 | 0.015 | 0 | 0.015 | 0 | 0.015 | 0 | 0.015 | 0 | 0.015 | 0 | 0.015 |
| pDA332 | | | 0.2 | 0.2 | 0.04 | 0.04 | 0.04 | 0.04 | 0.04 | 0.04 | 0.04 | 0.04 |
| pDA320 | | | | | 0.2 | 0.2 | 0.2 | 0.2 | 0.2 | 0.2 | 0.2 | 0.2 |
| pDA315 | | | | | | | 0.34 | 0.34 | 0.34 | 0.34 | 0.34 | 0.34 |
| pDA335 | | | | | | | | | 0.13 | 0.13 | 0.13 | 0.13 |
| pDA336 | | | | | | | | | | | 0.093 | 0.093 |
| pDA306 | 10 | 10 | | | | | | | | | | |
| pDA303 | | | 10 | 10 | | | | | | | | |
| pDA304 | | | | | 10 | 10 | | | | | | |
| pDA309 | | | | | | | 10 | 10 | | | | |
| pDA307 | | | | | | | | | 10 | 10 | | |
| pDA305 | | | | | | | | | | | 10 | 10 |

TABLE 6
Component sequences (elements alternating bold and italic)

pRC011: J23107.Buj.MCP-SYNZIP6 (SEQ ID NO: 12)
tttacggctagctcagccctaggtattatgctagcGAATTCATTAAAGAGGAGAAAGGTACCatggggcc
cgcttctaactttactcagttcgttctcgtcgacaatggcggaactggcgacgtgactgtcgccccaagcaacttcgctaac
gggatcgctgaatggatcagctctaactcgcgttcacaggcttacaaagtaacctgtagcgttcgtcagagctctgcgca
gaatcgcaaatacaccatcaaagtcgaggtgcctaaaggcgcctggcgttcgtacttaaatatggaactaaccattccaa
ttttcgccacgaattccgactgcgagcttattgttaaggcaatgcaaggtctcctaaaagatggaaacccgattccctcag
caatcgcagcaaactccggcatctacGGTGGCGGAGGTAGCCAAAAAGTTGCGCAGCTGAAA
AACCGTGTTGCGTACAAACTGAAAGAAAACGCGAAGCTGGAGAACATCGTGGCGC
GTCTGGAAAACGACAATGCGAACCTGGAGAAAGACATTGCGAATCTCGAAAAGGAC
ATCGCAAATCTGGAACGTGACGTTGCGCGTTAAGCGGCCGCcacgcaaaaaaccccgcttc
ggcggggttttttcgc
pRC012: J23107.Buj.SoxS-SYNZIP5 (SEQ ID NO: 13)
tttacggctagctcagccctaggtattatgctagcGAATTCATTAAAGAGGAGAAAGGTACCATG
TCCCATCAGAAAATTATTCAGGATCTTATCGCATGGATTGACGAGCATATTGACCAGCCGCTTAA
CATTGATGTAGTCGCAAAAAAATCAGGCTATTCAAAGTGGTACTTGCAACGAATGTTCCGCACGG
TGACGCATCAGACGCTTGGCGATTACATTCGCCAACGCCGCCTGTTACTGGCCGCCGTTGAGTTG
CGCACCACCGAGCGTCCGATTTTTGATATCGCAATGGACCTGGGTTATGTCTCGCAGCAGACCTT
CTCCCGCGTTTTCGCGCGGCAGTTTGATCGCACTCCCGCGGATTATCGCCACCGCCTGGGTGGCG
GAGGTAGCAACACCGTTAAAGAACTGAAAAACTACATCCAGGAGCTGGAAGAGCGTAACGCTGAA
CTCAAAAACCTGAAGGAACACCTGAAATTCGCAAAAGCGGAACTGGAATTCGAACTGGCGGCTCA
CAAATTCGAGTAAGGCGCGCCcacgcaaaaaaccccgcttcggcggggttttttcgc
pRC025: J23107.Buj.MCP-ABI (SEQ ID NO: 14)
tttacggctagctcagccctaggtattatgctagcGAATTCATTAAAGAGGAGAAAGGTACCatg
gggcccgcttctaactttactcagttcgttctcgtcgacaatggcggaactggcgacgtgactgt
cgccccaagcaacttcgctaacgggatcgctgaatggatcagctctaactcgcgttcacaggctt
acaaagtaacctgtagcgttcgtcagagctctgcgcagaatcgcaaatacaccatcaaagtcgag
gtgcctaaaggcgcctggcgttcgtacttaaatatggaactaaccattccaattttcgccacgaa
ttccgactgcgagcttattgttaaggcaatgcaaggtctcctaaaagatggaaacccgattccct
cagcaatcgcagcaaactccggcatctacGGTGGCGGAGGTAGCACGCGTGTGCCTTTGTATGGT
TTTACTTCGATTTGTGGAAGAAGACCTGAGATGGAAGCTGCTGTTTCGACTATACCAAGATTCCT
TCAATCTTCCTCTGGTTCGATGTTAGATGGTCGGTTTGATCCTCAATCCGCCGCTCATTTCTTCG
GTGTTTACGACGGCCATGGCGGTTCTCAGGTAGCGAACTATTGTAGAGAGAGGATGCATTTGGCT
TTGGCGGAGGAGATAGCTAAGGAGAAACCGATGCTCTGCGATGGTGATACGTGGCTGGAGAAGTG
GAAGAAAGCTCTTTTCAACTCGTTCCTGAGAGTTGACTCGGAGATTGAGTCAGTTGCGCCGGAGA
CGGTTGGGTCAACGTCGGTGGITGCCGTTGTTTTCCCGTCTCACATCTTCGTCGCTAACTGCGGT
GACTCTAGAGCCGTTCTTTGCCGCGGCAAAACTGCACTTCCATTATCCGTTGACCATAAACCGGA
TAGAGAAGATGAAGCTGCGAGGATTGAAGCCGCAGGAGGGAAAGTGATTCAGTGGAATGGAGCTC
GTGTTTTCGGTGTTCTCGCCATGTCGAGATCCATTGGCGATAGATACTTGAAACCATCCATCATT
CCTGATCCGGAAGTGACGGCTGTGAAGAGAGTAAAAGAAGATGATTGTCTGATTTTGGCGAGTGA
CGGGGTTTGGGATGTAATGACGGATGAAGAAGCGTGTGAGATGGCAAGGAAGCGGATTCTCTTGT
GGCACAAGAAAAACGCGGTGGCTGGGGATGCATCGTTGCTCGCGGATGAGCGGAGAAAGGAAGGG
AAAGATCCTGCGGCGATGTCCGCGGCTGAGTATTTGTCAAAGCTGGCGATACAGAGAGGAAGCAA
AGACAACATAAGTGTGGTGGTGGTTGATTTGAAGTAAGGCGCGCCcacgcaaaaaaccccgcttc
ggcggggttttttcgc
pRC027: J23107.Buj.SoxS-PYL1 (SEQ ID NO: 15)
tttacggctagctcagccctaggtattatgctagcGAATTCATTAAAGAGGAGAAAGGTACCATG
TCCCATCAGAAAATTATTCAGGATCTTATCGCATGGATTGACGAGCATATTGACCAGCCGCTTAA
CATTGATGTAGTCGCAAAAAAATCAGGCTATTCAAAGTGGTACTTGCAACGAATGTTCCGCACGG
TGACGCATCAGACGCTTGGCGATTACATTCGCCAACGCCGCCTGTTACTGGCCGCCGTTGAGTTG
CGCACCACCGAGCGTCCGATTTTTGATATCGCAATGGACCTGGGTTATGTCTCGCAGCAGACCTT
CTCCCGCGTTTTCGCGCGGCAGTTTGATCGCACTCCCGCGGATTATCGCCACCGCCTGGGTGGCG
GAGGTAGCatgggtgggggcgcgccaactcaagacgaattcacccaactctcccaatcaatcgcc
gagttccacacgtaccaactcggtaacggccgttgctcatctctcctagctcagcgaatccacgc
gccgccggaaacagtatggtccgtggtgagacgtttcgataggccacagatttacaaacacttca
tcaaaagctgtaacgtgagtgaagatttcgagatgcgagtgggatgcacgcgcgacgtgaacgtg
ataagtggattaccggcgaatacgtctcgagagagattagatctgttggacgatgatcggagagt
gactgggtttagtataaccggtggtgaacataggctgaggaattataaatcggttacgacggttc
atagatttgagaaagaagaagaagaagaaaggatctggaccgttgttttggaatcttatgttgtt
gatgtaccggaaggtaattcggaggaagatacgagattgtttgctgatacggttattagattgaa
tcttcagaaacttgcttcgatcactgaagctatgaacTAAGCGGCCGCCcgcaaaaaaccccgct
tcggcggggttttttcgc
pRC042: J23107.Buj.MCP-GAI (SEQ ID NO: 16)
tttacggctagctcagccctaggtattatgctagcGAATTCATTAAAGAGGAGAAAGGTACCatg
gggcccgcttctaactttactcagttcgttctcgtcgacaatggcggaactggcgacgtgactgt
cgccccaagcaacttcgctaacgggatcgctgaatggatcagctctaactcgcgttcacaggctt
acaaagtaacctgtagcgttcgtcagagctctgcgcagaatcgcaaatacaccatcaaagtcgag
gtgcctaaaggcgcctggcgttcgtacttaaatatggaactaaccattccaattttcgccacgaa
ttccgactgcgagcttattgttaaggcaatgcaaggtctcctaaaagatggaaacccgattccct
cagcaatcgcagcaaactccggcatctacGGTGGCGGAGGTAGCATGAAGCGCGATCATCATCAC
CACCACCACCAGGATAAAAAGACGATGATGATGAATGAGGAAGATGATGGAAACGGGATGGACGA
ATTGCTGGCAGTGCTGGGATATAAGGTGCGTTCGTCCGAAATGGCAGATGTTGCTCAGAAATTGG
AGCAGTTAGAAGTAATGATGAGTAACGTTCAAGAAGATGATCTTTCACAGTTAGCGACCGAAACT
GTCCACTACAACCCTGCTGAGCTTTACACTTGGTTGGACTCCATGCTTACCGATCTTAACtgacg
caaaaaaccccgcttcggcggggttttttcgc
pRC043: J23107.Buj. SoxS-GID1 (SEQ ID NO: 17)
tttacggctagctcagccctaggtattatgctagcGAATTCATTAAAGAGGAGAAAGGTACCATG
TCCCATCAGAAAATTATTCAGGATCTTATCGCATGGATTGACGAGCATATTGACCAGCCGCTTAA
CATTGATGTAGTCGCAAAAAAATCAGGCTATTCAAAGTGGTACTTGCAACGAATGTTCCGCACGG
TGACGCATCAGACGCTTGGCGATTACATTCGCCAACGCCGCCTGTTACTGGCCGCCGTTGAGTTG
CGCACCACCGAGCGTCCGATTTTTGATATCGCAATGGACCTGGGTTATGTCTCGCAGCAGACCTT
CTCCCGCGTTTTCGCGCGGCAGTTTGATCGCACTCCCGCGGATTATCGCCACCGCCTGGGTGGCG
GAGGTAGCATGGCAGCCTCCGACGAGGTAAATCTTATTGAGAGTCGTACCGTCGTTCCCTTGAAT
ACTTGGGTGTTGATCTCGAATTTCAAGGTCGCGTACAATATCTTACGCCGCCCGGATGGAACCTT
TAACCGTCACCTTGCAGAATATCTGGACCGCAAAGTTACAGCAAATGCTAATCCAGTTGACGGTG
TTTTCAGTTTTGACGTGCTGATTGATCGCCGTATCAACCTTCTGTCCCGTGTCTATCGTCCTGCT
TACGCCGATCAGGAGCAACCTCCATCCATTCTGGATCTGGAAAAACCAGTGGATGGGGACATTGT
CCCTGTCATCCTTTTTTTCCACGGGGGGTCGTTCGCCCACTCGTCCGCCAACAGTGCGATCTACG
ACACTTTATGTCGTCGTCTTGTCGGTCTTTGCAAATGCGTGGTCGTTTCCGTGAATTACCGTCGC
GCTCCGGAGAACCCCTACCCATGTGCCTACGACGACGGATGGATTGCGTTAAATTGGGTTAATTC
ACGTAGCTGGCTGAAAAGCAAGAAAGATTCGAAGGTTCACATTTTTTTAGCGGGCGATTCTTCAG
GAGGGAACATCGCTCATAATGTCGCATTGCGTGCAGGAGAGTCTGGCATCGATGTTCTGGGCAAC
ATTTTACTGAACCCGATGTTTGGGGGGAACGAGCGCACAGAATCCGAGAAAAGCTTGGACGGGAA
GTATTTCGTGACTGTTCGCGATCGTGACTGGTATTGGAAAGCGTTCTTGCCCGAGGGAGAGGACC
GCGAGCACCCCGCATGCAACCCCTTTTCACCTCGCGGAAAATCGCTGGAGGGGGTCAGTTTCCCA
AAATCTTTAGTCGTAGTAGCTGGCCTGGATCTGATCCGTGATTGGCAACTTGCGTATGCTGAAGG
CCTTAAGAAGGCTGGTCAAGAAGTAAAGCTGATGCACTTAGAGAAAGCTACGGTTGGCTTTTATC
TGTTACCAAATAACAATCACTTCCATAATGTGATGGATGAGATCTCCGCTTTCGTTAATGCGGAA
TGCtgacgcaaaaaaccccgcttcggcggggttttttcgc
pDA010.EL222: J23106.BBaB0034.EL222 (SEQ ID NO: 18)
TTTACGGCTAGCTCAGTCCTAGGTATAGTGCTAGCCTAGAGAAAGAGGAGAAATACTAGATGTTG
GATATGGGACAAGATCGGCCGATCGATGGAAGTGGGGCACCCGGGGCAGACGACACACGCGTTGA
GGTGCAACCGCCGGCGCAGTGGGTCCTCGACCTGATCGAGGCCAGCCCGATCGCATCGGTCGTGT
CCGATCCGCGTCTCGCCGACAATCCGCTGATCGCCATCAACCAGGCCTTCACCGACCTGACCGGC
TATTCCGAAGAAGAATGCGTCGGCCGCAATTGCCGATTCCTGGCAGGTTCCGGCACCGAGCCGTG
GCTGACCGACAAGATCCGCCAAGGCGTGCGCGAGCACAAGCCGGTGCTGGTCGAGATCCTGAACT
ACAAGAAGGACGGCACGCCGTTCCGCAATGCCGTGCTCGTTGCACCGATCTACGATGACGACGAC
GAGCTTCTCTATTTCCTCGGCAGCCAGGTCGAAGTCGACGACGACCAGCCCAACATGGGCATGGC
GCGCCGCGAACGCGCCGCGGAAATGCTCAAGACGCTGTCGCCGCGCCAGCTCGAGGTTACGACGC
TGGTGGCATCGGGCTTGCGCAACAAGGAAGTGGCGGCCCGGCTCGGCCTGTCGGAGAAAACCGTC
AAGATGCACCGCGGGCTGGTGATGGAAAAGCTCAACCTGAAGACCAGCGCCGATCTGGTGCGCAT
TGCCGTCGAAGCCGGAATCTAAGGATCCAAACTCGAGTAAGGATCTCCAGGCATCAAATAAAACG
AAAGGCTCAGTCGAAAGACTGGGCCTTTCGTTTTATCTGTTGTTTGTCGGTGAACGCTCTCTACT
AGAGTCACACTGGCTCACCTTCGGGTGGGCCTTTCTGCGTTTATA
pDA040.BLD7-mRFP: EL222_Binding_region.D7.Bujard.mRFP (SEQ ID NO: 19)
GGTAGCCTTTAGTCCATGTTGACGGTGAAGAGTATCAGAGGGATTGTGCTAGCGAATTCATTAAA
GAGGAGAAAGGTACCATGGCGAGTAGCGAAGACGTTATCAAAGAGTTCATgcgtttcaaagttcg
tatggaaggttccgttaacggtcacgagttcgaaatcgaaggtgaaggtgaaggtcgtccgtacg
aaggtacccagaccgctaaactgaaagttaccaaaggtggtccgctgccgttcgcttgggacatc
ctgtccccgcagttccagtacggttccaaagcttacgttaaacacccggctgacatcccggacta
cctgaaactgtccttcccggaaggtttcaaatgggaacgtgttatgaacttcgaagacggtggtg
ttgttaccgttacccaggactcctccctgcaagacggtgagttcatctacaaagttaaactgcgt
ggtaccaacttcccgtccgacggtccggttatgcagaaaaaaaccatgggttgggaagcttccac
cgaacgtatgtacccggaagacggtgctctgaaaggtgaaatcaaaatgcgtctgaaactgaaag
acggtggtcactacgacgctgaagttaaaaccacctacatggctaaaaaaccggttcagctgccg
ggtgcttacaaaaccgacatcaaactggacatcacctcccacaacgaagactacaccatcgttga
acagtacgaacgtgctgaaggtcgtcactccaccggtgcttaaggatccaaactcgagtaaggat
ctGTGCTTTTTTTaacgcatgagAAAGCCCCCGGAAGATCACCTTCCGGGGGCTTTtttattgcg
c

TABLE 7
ANOVA analysis of combinatorial promoter screens.

| | Sum of | Degrees of | F | p |
|---|---|---|---|---|
| | Squares | Freedom | Value | Value |
| Minimal | 23155.9 | 3 | 9.5 | 1.2e−4 |
| Promoter | | | | |
| UP-Element | 315527.0 | 3 | 129.7 | 5.5e−18 |
| Minimal | 53678.2 | 9 | 7.4 | 1.0e−5 |
| Promoter & | | | | |
| UP-Element | | | | |
| Residual | 25954.2 | 32 | — | — |

TABLE 8
Nucleotide symbols and their corresponding
meanings as represented in the Listing.

| Symbol | Meaning |
|---|---|
| A | Adenine |
| G | Guanine |
| C | Cytosine |
| T | Thymine |
| U | Uracil |
| R | Purine (A or G) |
| Y | Pyrimidine (C or T) |
| N | Any nucleotide |
| W | Weak (A or T) |
| S | Strong (G or C) |
| M | Amino (A or C) |
| K | Keto (G or T) |
| B | Not A (G or C or T) |
| H | Not G (A or C or T) |
| D | Not C (A or G or T) |
| V | Not T (A or G or C) |

TABLE 9
Example reference sequences.

| Name | Sequence |
|---|---|
| J23117 | TTGACAGCTAGCTCAGTCCTAGGGATTGTGCT |
| Minimal | AGC (SEQ ID NO: 83) |
| Promoter | |
| Synthetic J3 | CGTCGTCTTGAAGTTGCGATTATAGA |
| UP-element | (SEQ ID NO: 84) |
| Synthetic J306 | ACGGAGCGTTCTGGACACAA |
| guide RNA | (SEQ ID NO: 85) |
| target | |
| Natural luxI | TTAGCGAAGAAAATGGTTTGTTATAGTCGAAT |
| minimal | AAA (SEQ ID NO: 86) |
| promoter | |

### NON-LIMITING EMBODIMENTS

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. A promoter comprising a minimal promoter sequence and an upstream element (UP-element) sequence that comprises a nucleotide base mutation for a modified promoter-RNA polymerase interaction to configure the promoter for a low basal transcription and a high active transcription.

Embodiment 2. The promoter of any other Embodiment, wherein the UP-element sequence comprises a higher GC content relative to a GC content of an UP-element reference sequence.

Embodiment 3. The promoter of any other Embodiment, wherein the UP-element sequence comprises between 50% and 100% GC content.

Embodiment 4. The promoter of any other Embodiment, wherein: the UP-element sequence comprises at least 80% identity with a sequence selected from SEQ ID NOs: 33-49; and the minimal promoter sequence comprises at least 80% identity with a sequence selected from SEQ ID NOs: 50-66.

Embodiment 5. The promoter of any other Embodiment, wherein: the UP-element sequence comprises at least 90% identity with a sequence selected from SEQ ID NOs: 33-49; and the minimal promoter sequence comprises at least 90% identity with a sequence selected from SEQ ID NOs: 50-66.

Embodiment 6. The promoter of any other Embodiment, wherein: the UP-element sequence comprises 100% identity with a sequence selected from SEQ ID NOs: 33-49; and the minimal promoter sequence comprises 100% identity with a sequence selected from SEQ ID NOs: 50-66.

Embodiment 7. The promoter of any other Embodiment, further comprising a scaffold RNA (scRNA) target sequence configured to bind a guide RNA (gRNA) or single guide RNA (sgRNA) for localization of a Clustered Regularly Interspaced Short Palindromic Repeats activation (CRISPRa) complex to the promoter, wherein the CRISPRa complex comprises a deactivated Cas9 (dCas9) linked with a MS2 bacteriophage coat protein (MCP) polypeptide and a first heterodimer interaction domain.

Embodiment 8. The promoter of any other Embodiment, wherein the scRNA target sequence comprises a higher GC content relative to a GC content of a scRNA reference sequence.

Embodiment 9. The promoter of any other Embodiment, wherein: the scRNA target sequence comprises at least 80% identity with a sequence selected from SEQ ID NOs: 20-32.

Embodiment 10. The promoter of any other Embodiment, wherein: the scRNA target sequence comprises at least 90% identity with a sequence selected from SEQ ID NOs: 20-32.

Embodiment 11. The promoter of any other Embodiment, wherein: the scRNA target sequence comprises 100% identity with a sequence selected from SEQ ID NOs: 20-32.

Embodiment 12. The promoter of any other Embodiment, wherein the promoter comprises at least 80% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

Embodiment 13. The promoter of any other Embodiment, wherein the promoter comprises at least 90% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

Embodiment 14. The promoter of any other Embodiment, wherein the promoter comprises 100% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

Embodiment 15. A CRISPRa/i node, comprising: the promoter of any other Embodiment; a dCas9 polypeptide complexed, via a gRNA or a sgRNA, with a MCP polypeptide that is linked with a first heterodimer interaction domain; and a second heterodimer interaction domain that is linked with a SRY-related HMG-box (SoxS) domain; wherein the gRNA or the sgRNA is configured to interact with the promoter to localize the first heterodimer interaction domain to the promoter, and wherein the first heterodimer interaction domain is configured to conditionally interact with the second heterodimer interaction domain to localize the SoxS domain to the promoter.

Embodiment 16. The CRISPRa/i node of any other Embodiment, wherein a condition is associated with an interaction between the first heterodimer interaction domain and the second heterodimer interaction domain, and wherein the condition is met the interaction occurs and an activation complex is formed for activation of transcription via the SoxS domain.

Embodiment 17. The CRISPRa/i node of any other Embodiment, wherein the condition comprises irradiation of a matrix that comprises the CRISPRa/i node with a light that comprises a blue light element or contact of the matrix that comprises the CRISPRa/i node with a small molecule.

Embodiment 18. The CRISPRa/i node of any other Embodiment, wherein the condition comprises irradiation of the matrix with the light that comprises the blue light element and the method is performed using a EL222 light-responsive promoter system.

Embodiment 19. The CRISPRa/i node of any other Embodiment, wherein: the first heterodimer interaction domain comprises a heterospecific synthetic coiled-coil peptide A (SYNZIP A) domain and the second heterodimer interaction domain comprises a heterospecific synthetic coiled-coil peptide B (SYNZIP B) domain or the first heterodimer interaction domain comprises a SYNZIP B domain and the second heterodimer interaction domain comprises a SYNZIP A domain; the first heterodimer interaction domain comprises an abortive infectivity (ABI) domain and the second heterodimer interaction domain comprises a pyrabactin resistance-like 1 (PYL1) domain or the first heterodimer interaction domain comprises a PYL1 domain and the second heterodimer interaction domain comprises an ABI domain; or the first heterodimer interaction domain comprises a gibberellin (GAI) domain and the second heterodimer interaction domain comprises a gibberellin insensitive dwarf 1 (GID1) domain or the first heterodimer interaction domain comprises a GID1 domain and the second heterodimer interaction domain comprises a GAI domain.

Embodiment 20. The CRISPRa/i node of any other Embodiment, wherein the UP-element sequence comprises between 50% and 100% GC content.

Embodiment 21. The CRISPRa/i node of any other Embodiment, wherein: the UP-element sequence comprises at least 80% identity with a sequence selected from SEQ ID NOs: 33-49; and the minimal promoter sequence comprises at least 80% identity with a sequence selected from SEQ ID NOs: 50-66.

Embodiment 22. The CRISPRa/i node of any other Embodiment, wherein: the UP-element sequence comprises at least 90% identity with a sequence selected from SEQ ID NOs: 33-49; and the minimal promoter sequence comprises at least 90% identity with a sequence selected from SEQ ID NOs: 50-66.

Embodiment 23. The CRISPRa/i node of any other Embodiment, wherein: the UP-element sequence comprises 100% identity with a sequence selected from SEQ ID NOs: 33-49; and the minimal promoter sequence comprises 100% identity with a sequence selected from SEQ ID NOs: 50-66.

Embodiment 24. The CRISPRa/i node of any other Embodiment, further comprising a scRNA target sequence configured to bind a gRNA or sgRNA for localization of a CRISPRa complex to the promoter, wherein the CRISPRa complex comprises a dCas9 linked with a MCP polypeptide and a first heterodimer interaction domain.

Embodiment 25. The CRISPRa/i node of any other Embodiment, wherein the scRNA target sequence comprises a higher GC content relative to a GC content of a scRNA target reference sequence.

Embodiment 26. The CRISPRa/i node of any other Embodiment, wherein: the scRNA target sequence comprises at least 80% identity with a sequence selected from SEQ ID NOs: 20-32.

Embodiment 27. The CRISPRa/i node of any other Embodiment, wherein: the scRNA target sequence comprises at least 90% identity with a sequence selected from SEQ ID NOs: 20-32.

Embodiment 28. The CRISPRa/i node of any other Embodiment, wherein: the scRNA target sequence comprises 100% identity with a sequence selected from SEQ ID NOs: 20-32.

Embodiment 29. The CRISPRa/i node of any other Embodiment, wherein the promoter comprises at least 80% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

Embodiment 30. The CRISPRa/i node of any other Embodiment, wherein the promoter comprises at least 90% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

Embodiment 31. The CRISPRa/i node of any other Embodiment, wherein the promoter comprises 100% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

Embodiment 32. A synthetic gene regulatory network (GRN) comprising at least a first CRISPRa/i node and a second CRISPRa/i node, wherein the first CRISPRa/i node comprises a first promoter of any other Embodiment and the second CRISPRa/i node comprises a second promoter of any other Embodiment, wherein the first CRISPRa/i node is configured for a sensing function and the second CRISPRa/i node is configured for a propagation function or a reporting function.

Embodiment 33. The synthetic GRN of any other Embodiment, wherein a first transcription product results from activation of transcription of the first CRISPRa/i node and is an input to the second CRISPRa/i node, and wherein a second transcription product results from activation of transcription of the second CRISPRa/i node and is an output of the second CRISPRa/i node, the synthetic GRN, or both.

Embodiment 34. The synthetic GRN of any other Embodiment, wherein the synthetic GRN comprises a feedback loop, a feedforward loop, a positive feedback, a negative feedback, a positive feedforward, and/or a negative feedforward.

Embodiment 35. The synthetic GRN of any other Embodiment, wherein the propagation function implements a decreasing strategy, a constant strategy, an increasing strategy, or a data-informed strategy.

Embodiment 36. The synthetic GRN of any other Embodiment, wherein the first promoter, the second promoter, or both, comprises a minimal promoter sequence and an UP-element sequence that comprises a nucleotide base mutation for a modified promoter-RNA polymerase interaction to configure the promoter for a low basal transcription and a high active transcription, and wherein the first CRISPRa/i node, the second CRISPRa/i node, or both, comprises: a dCas9 polypeptide complexed, via a gRNA or a sgRNA, with a MCP polypeptide that is linked with a first heterodimer interaction domain; and a second heterodimer interaction domain that is linked with a SoxS domain; wherein the gRNA or the sgRNA is configured to interact with the first promoter, the second promoter, or both to localize the first heterodimer interaction domain to the first promoter, the second promoter, or both, and wherein the first heterodimer interaction domain is configured to conditionally interact with the second heterodimer interaction domain to localize the SoxS domain to the first promoter, the second promoter, or both.

Embodiment 37. The synthetic GRN of any other Embodiment, wherein a condition is associated with an interaction between the first heterodimer interaction domain and the second heterodimer interaction domain, and wherein the condition is met the interaction occurs and an activation complex is formed for activation of transcription via the SoxS domain.

Embodiment 38. The synthetic GRN of any other Embodiment, wherein the condition comprises irradiation of a matrix that comprises the CRISPRa/i node with a light that comprises a blue light element or contact of the matrix that comprises the CRISPRa/i node with a small molecule.

Embodiment 39. The synthetic GRN of any other Embodiment, wherein the condition comprises irradiation of the matrix with the light that comprises the blue light element and the method is performed using a EL222 light-responsive promoter system.

Embodiment 40. The synthetic GRN of any other Embodiment, wherein: the first heterodimer interaction domain comprises a SYNZIP A domain and the second heterodimer interaction domain comprises a SYNZIP B domain or the first heterodimer interaction domain comprises a SYNZIP B domain and the second heterodimer interaction domain comprises a SYNZIP A domain; the first heterodimer interaction domain comprises an ABI domain and the second heterodimer interaction domain comprises a PYL1 domain or the first heterodimer interaction domain comprises a PYL1 domain and the second heterodimer interaction domain comprises an ABI domain; or the first heterodimer interaction domain comprises a GAI domain and the second heterodimer interaction domain comprises a GID1 domain or the first heterodimer interaction domain comprises a GID1 domain and the second heterodimer interaction domain comprises a GAI domain.

Embodiment 41. The synthetic GRN of any other Embodiment, wherein the UP-element sequence comprises between 50% and 100% GC content.

Embodiment 42. The synthetic GRN of any other Embodiment, wherein: the UP-element sequence comprises at least 80% identity with a sequence selected from SEQ ID NOs: 33-49; and the minimal promoter sequence comprises at least 80% identity with a sequence selected from SEQ ID NOs: 50-66.

Embodiment 43. The synthetic GRN of any other Embodiment, wherein: the UP-element sequence comprises at least 90% identity with a sequence selected from SEQ ID NOs: 33-49; and the minimal promoter sequence comprises at least 90% identity with a sequence selected from SEQ ID NOs: 50-66.

Embodiment 44. The synthetic GRN of any other Embodiment, wherein: the UP-element sequence comprises 100% identity with a sequence selected from SEQ ID NOs: 33-49; and the minimal promoter sequence comprises 100% identity with a sequence selected from SEQ ID NOs: 50-66.

Embodiment 45. The synthetic GRN of any other Embodiment, further comprising a scRNA target sequence configured to bind the gRNA or the sgRNA for localization of a CRISPRa complex to the promoter, wherein the CRISPRa complex comprises the dCas9 polypeptide complexed, via the gRNA or the sgRNA, with the MCP polypeptide that is linked with the first heterodimer interaction domain.

Embodiment 46. The synthetic GRN of any other Embodiment, wherein the scRNA target sequence comprises a higher GC content relative to a GC content of a scRNA reference sequence.

Embodiment 47. The synthetic GRN of any other Embodiment, wherein: the scRNA target sequence comprises at least 80% identity with a sequence selected from SEQ ID NOs: 20-32.

Embodiment 48. The synthetic GRN of any other Embodiment, wherein: the scRNA target sequence comprises at least 90% identity with a sequence selected from SEQ ID NOs: 20-32.

Embodiment 49. The synthetic GRN of any other Embodiment, wherein: the scRNA target sequence comprises 100% identity with a sequence selected from SEQ ID NOs: 20-32.

Embodiment 50. The synthetic GRN of any other Embodiment, wherein the first promoter, the second promoter, or both, comprises at least 80% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

Embodiment 51. The synthetic GRN of any other Embodiment, wherein the first promoter, the second promoter, or both, comprises at least 90% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

Embodiment 52. The synthetic GRN of any other Embodiment, wherein the first promoter, the second promoter, or both, comprises 100% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

Embodiment 53. The synthetic GRN of any other Embodiment, wherein the first promoter has a higher dynamic range of transcription and the second promoter has a higher absolute range of transcription.

Embodiment 54. The synthetic GRN of any other Embodiment, wherein the synthetic GRN is at least one, two, three, four, five, or six layers deep, and is at least one, two, three, or four branches wide.

Embodiment 55. One or more nucleic acids comprising nucleotide sequences encoding one or more polypeptides of any other Embodiment, the gRNA or the sgRNA of any other Embodiment, one or more genes regulated by the CRISPRa/i node of any other Embodiment, the promoter of any other Embodiment, or any combination thereof.

Embodiment 56. An E. coli CFS comprising polypeptides of any other Embodiment and the promoter of any other Embodiment.

Embodiment 57. A genetically modified host cell comprising one or more nucleic acids encoding the promoter of any other Embodiment.

Embodiment 58. The genetically modified host cell of any other Embodiment, wherein the genetically modified host cell is a prokaryotic cell or a eukaryotic cell.

Embodiment 59. A synthetic biosensor, synthetic bioreactor, or other synthetic biological system comprising the synthetic GRN of any other Embodiment.

Embodiment 60. A method for control of transcription, the method comprising: providing a promoter of any other Embodiment; providing a first dimer interaction domain that is linked with a domain configured for interaction with the promoter; providing a second dimer interaction domain that is linked with a domain configured for interaction with an RNA polymerase; and meeting a condition that is associated with an interaction between the first dimer interaction domain and the second dimer interaction domain, wherein the condition is met the interaction occurs and an activation complex is formed for activation of transcription.

Embodiment 61. The method of any other Embodiment, wherein the providing the promoter comprises producing the promoter by: mutagenizing one or more nucleotide sequences of a reference promoter to produce one or more mutagenic libraries each comprising a plurality of candidate promoters; cloning the plurality of candidate promoters of the one or more mutagenic libraries onto a plurality of inducible reporter constructs; transforming the plurality of inducible reporter constructs into one or more host cells; measuring a basal level of transcription of the plurality of inducible reporter constructs by the one or more host cells; and measuring an activated level of transcription of the plurality of inducible reporter constructs by the one or more host cells; wherein a difference between the activated level of transcription and the basal level of transcription suggests that the promoter has been produced.

Embodiment 62. The method of any other Embodiment, wherein providing the first and second dimer interaction domains comprises providing: a non-dimerized EL222 transcription factor that comprises a first homodimer interaction domain and a second homodimer interaction domain as the first and second dimer interaction domains, respectively; or a CRISPRa complex that comprises a dCas9 polypeptide complexed, via a gRNA or a sgRNA, with a MCP polypeptide that is linked with a first heterodimer interaction domain, and a second heterodimer interaction domain that is linked with a SoxS domain, wherein the first heterodimer interaction domain is the first dimer interaction domain and the second heterodimer interaction domain is the second dimer interaction domain.

Embodiment 63. The method of any other Embodiment, wherein the condition comprises: irradiation of a matrix that comprises the non-dimerized EL222 transcription factor with a light that comprises a blue light element for homodimerization of the non-dimerized EL222 transcription factor, or contact of a matrix that comprises the CRISPRa complex with a small molecule for heterodimerization of the CRISPRa complex.

Embodiment 64. The method of any other Embodiment, wherein: the first heterodimer interaction domain comprises a SYNZIP A domain and the second heterodimer interaction domain comprises a SYNZIP B domain or the first heterodimer interaction domain comprises a SYNZIP B domain and the second heterodimer interaction domain comprises a SYNZIP A domain; the first heterodimer interaction domain comprises an ABI domain and the second heterodimer interaction domain comprises a PYL1 domain or the first heterodimer interaction domain comprises a PYL1 domain and the second heterodimer interaction domain comprises an ABI domain; or the first heterodimer interaction domain comprises a GAI domain and the second heterodimer interaction domain comprises a GID1 domain or the first heterodimer interaction domain comprises a GID1 domain and the second heterodimer interaction domain comprises a GAI domain.

Embodiment 65. A method for producing the promoter of any other Embodiment, the method comprising: selecting a reference promoter; mutagenizing one or more nucleotide sequences of the reference promoter to produce one or more mutagenic libraries each comprising a plurality of candidate promoters; cloning the plurality of candidate promoters of the one or more mutagenic libraries onto a plurality of inducible reporter constructs; transforming the plurality of inducible reporter constructs into one or more host cells; measuring a basal level of transcription of the plurality of inducible reporter constructs by the one or more host cells; and measuring an activated level of transcription of the plurality of inducible reporter constructs by the one or more host cells.

Embodiment 66. The method of any other Embodiment, wherein: a difference between the activated level of transcription and the basal level of transcription suggests that the promoter has either a higher dynamic range of transcription or a higher absolute range of transcription relative to corresponding metrics of the reference promoter.

Embodiment 67. A method for producing a heterodimer CRISPRa system, the method comprising: providing the promoter of any other Embodiment; providing a first heterodimer domain linked to a MCP polypeptide; providing a second heterodimer domain linked to a SoxS domain; modifying a gRNA or a sgRNA; and testing the gRNA or the sgRNA for an ability of the gRNA or the sgRNA to activate transcription in combination with the promoter, the first heterodimer domain linked to the MCP polypeptide, and the second heterodimer domain linked to the SoxS domain; wherein the ability of the gRNA or the sgRNA to activate transcription is associated with a structure of the gRNA or the sgRNA and a target location within the promoter; wherein if the gRNA or the sgRNA sufficiently activates transcription in combination with the high-performance activatable promoter, the first heterodimer domain linked to the MCP polypeptide, and the second heterodimer domain linked to the SoxS domain, the heterodimer CRISPRa system is produced.

Embodiment 68. The method of any other Embodiment, further comprising improving the structure of the gRNA or the sgRNA with the target location within the high-performance activatable promoter to improve the ability of the gRNA or the sgRNA to activate transcription.

Embodiment 69. The method of any other Embodiment, wherein the providing the first heterodimer domain linked to the MCP polypeptide comprises linking the first heterodimer domain to the MCP polypeptide and the providing the second heterodimer domain linked to the SoxS domain comprises linking the second heterodimer domain to the SoxS domain.

While illustrative embodiments have been illustrated and described, it will be appreciated that various changes can be made therein without departing from the spirit and scope of the disclosure.

1. A promoter comprising a minimal promoter sequence and an upstream element (UP-element) sequence that comprises a nucleotide base mutation for a modified promoter-RNA polymerase interaction to configure the promoter for a low basal transcription and a high active transcription.

2. The promoter of claim 1, wherein the UP-element sequence comprises a higher GC content relative to a GC content of an UP-element reference sequence.

3. The promoter of claim 2, wherein the UP-element sequence comprises between 50% and 100% GC content.

4. The promoter of claim 3, wherein:
the UP-element sequence comprises at least 80% identity with a sequence selected from SEQ ID NOs: 33-49; and
the minimal promoter sequence comprises at least 80% identity with a sequence selected from SEQ ID NOs: 50-66.

5-6. (canceled)

7. The promoter of claim 1, further comprising a scaffold RNA (scRNA) target sequence configured to bind a guide RNA (gRNA) or single guide RNA (sgRNA) for localization of a Clustered Regularly Interspaced Short Palindromic Repeats activation (CRISPRa) complex to the promoter, wherein the CRISPRa complex comprises a deactivated Cas9 (dCas9) linked with a MS2 bacteriophage coat protein (MCP) polypeptide and a first heterodimer interaction domain.

8. The promoter of claim 7, wherein the scRNA target sequence comprises a higher GC content relative to a GC content of a scRNA reference sequence.

9. The promoter of claim 8, wherein:
the scRNA target sequence comprises at least 80% identity with a sequence selected from SEQ ID NOs: 20-32.

10-11. (canceled)

12. The promoter of claim 1, wherein the promoter comprises at least 80% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65; or SEQ ID NOs: 32, 49, and 66.

13-14. (canceled)

15. A CRISPRa/i node, comprising:
a promoter comprising a minimal promoter sequence and an UP-element sequence that comprises a nucleotide base mutation for a modified promoter-RNA polymerase interaction to configure the promoter for a low basal transcription and a high active transcription;
a dCas9 polypeptide complexed, via a gRNA or a sgRNA, with a MCP polypeptide that is linked with a first heterodimer interaction domain; and
a second heterodimer interaction domain that is linked with a SRY-related HMG-box (SoxS) domain;
wherein the gRNA or the sgRNA is configured to interact with the promoter to localize the first heterodimer interaction domain to the promoter, and wherein the first heterodimer interaction domain is configured to conditionally interact with the second heterodimer interaction domain to localize the SoxS domain to the promoter.

16. The CRISPRa/i node of claim 15, wherein a condition is associated with an interaction between the first heterodimer interaction domain and the second heterodimer interaction domain, and wherein the condition is met the interaction occurs and an activation complex is formed for activation of transcription via the SoxS domain.

17. The CRISPRa/i node of claim 16, wherein the condition comprises irradiation of a matrix that comprises the CRISPRa/i node with a light that comprises a blue light element or contact of the matrix that comprises the CRISPRa/i node with a small molecule.

18. The CRISPRa/i node of claim 17, wherein the condition comprises irradiation of the matrix with the light that comprises the blue light element and the method is performed using a EL222 light-responsive promoter system.

19. The CRISPRa/i node of claim 15, wherein:
the first heterodimer interaction domain comprises a heterospecific synthetic coiled-coil peptide A (SYNZIP A) domain and the second heterodimer interaction domain comprises a heterospecific synthetic coiled-coil peptide B (SYNZIP B) domain or the first heterodimer interaction domain comprises a SYNZIP B domain and the second heterodimer interaction domain comprises a SYNZIP A domain;
the first heterodimer interaction domain comprises an abortive infectivity (ABI) domain and the second heterodimer interaction domain comprises a pyrabactin resistance-like 1 (PYL1) domain or the first heterodimer interaction domain comprises a PYL1 domain and the second heterodimer interaction domain comprises an ABI domain; or
the first heterodimer interaction domain comprises a gibberellin (GAI) domain and the second heterodimer interaction domain comprises a gibberellin insensitive dwarf 1 (GID1) domain or the first heterodimer interaction domain comprises a GID1 domain and the second heterodimer interaction domain comprises a GAI domain.

20. The CRISPRa/i node of claim 15, wherein the UP-element sequence comprises between 50% and 100% GC content.

21. The CRISPRa/i node of claim 20, wherein:
the UP-element sequence comprises at least 80% identity with a sequence selected from SEQ ID NOs: 33-49; and
the minimal promoter sequence comprises at least 80% identity with a sequence selected from SEQ ID NOs: 50-66.

22-25. (canceled)

26. The CRISPRa/i node of claim 20, wherein:
the scRNA target sequence comprises at least 80% identity with a sequence selected from SEQ ID NOs: 20-32.

27-28. (canceled)

29. The CRISPRa/i node of claim 15, wherein the promoter comprises at least 80% identity with: SEQ ID NOs: 20, 33, and 50; SEQ ID NOs: 21, 34, and 51; SEQ ID NOs: 22, 35, and 52; SEQ ID NOs: 23, 36, and 53; SEQ ID NOs: 24, 37, and 54; SEQ ID NOs: 25, 38, and 55; SEQ ID NOs: 26, 39, and 56; SEQ ID NOs: 27, 40, and 57; SEQ ID NOs: 28, 41, and 58; SEQ ID NOs: 42 and 59; SEQ ID NOs: 43 and 60; SEQ ID NOs: 44 and 61; SEQ ID NOs: 45 and 62; SEQ ID NOs: 29, 46, and 63; SEQ ID NOs: 30, 47, and 64; SEQ ID NOs: 31, 48, and 65;
or SEQ ID NOs: 32, 49, and 66.

30-54. (canceled)

55. One or more nucleic acids comprising nucleotide sequences encoding one or more polypeptides of claim 15, the gRNA or the sgRNA of claim 15, one or more genes regulated by the CRISPRa/i node of claim 15, the promoter of claim 15, or any combination thereof.

56. An E. coli CFS comprising polypeptides of claim 15 and the promoter of claim 15; or
a genetically modified host cell comprising one or more nucleic acids encoding the promoter of claim 15; or
a synthetic biosensor, synthetic bioreactor, or other synthetic biological system comprising the CRISPRa/i node of claim 15.

57-59. (canceled)

60. A method for control of transcription, the method comprising:
combining a promoter with a first polypeptide and a second polypeptide;
wherein the promoter comprises a sequence that comprises a nucleotide base mutation for a modified promoter-RNA polymerase interaction to configure the promoter for a low basal transcription and a high active transcription;
wherein the first polypeptide comprises a first dimer interaction domain that is linked with a domain configured for interaction with the promoter; and
wherein the second polypeptide comprises a second dimer interaction domain that is linked with a domain configured for interaction with an RNA polymerase; and
meeting a condition that is associated with an interaction between the first dimer interaction domain and the second dimer interaction domain, wherein the condition is met the interaction occurs and an activation complex is formed for activation of transcription.

61-69. (canceled)
